(19) United States
(12) Patent Application Publication
(10) Pub. No.: US 2026/0070927 A1
(43) Pub. Date: Mar. 12, 2026

(54) INDAZOLE MACROCYCLES FOR THE TREATMENT OF AUTOIMMUNE DISEASE

(71) Applicant: Hoffmann-La Roche Inc., Little Falls, NJ (US)

(72) Inventors: Xingchun HAN, Shanghai (CN); Min JIANG, Shanghai (CN); Buyu KOU, Shanghai (CN); Haixia LIU, Shanghai (CN); Yafei LIU, Shanghai (CN); Hong SHEN, Shanghai (CN); Zhenhong ZHANG, Shanghai (CN); Yao WU, Shanghai (CN); Zhiwei ZHANG, Shanghai (CN); Wei ZHU, Shanghai (CN)

(73) Assignee: Hoffmann-La Roche Inc., Little Falls, NJ (US)

(21) Appl. No.: 18/871,981

(22) PCT Filed: Jun. 5, 2023
(86) PCT No.: PCT/EP2023/064902

§ 371 Date: Dec. 5, 2024

(30) Foreign Application Priority Data
Jun. 6, 2022 (WO) PCT/CN2022/097158
Jan. 10, 2023 (WO) PCT/CN2023/071706

(51) Int. Cl. C07D 519/00; A61K 31/519; A61K 31/529; A61K 31/5377

(52) U.S. Cl. CPC C07D 519/00; A61K 31/519; A61K 31/529; A61K 31/5377

(57) ABSTRACT

The present invention relates to compounds of formula (Ia), (Ia), wherein R1 to R3, Q1, Q2, A1 to A6 are as described herein, and their pharmaceutically acceptable salt thereof, and compositions including the compounds and methods of using the compounds.

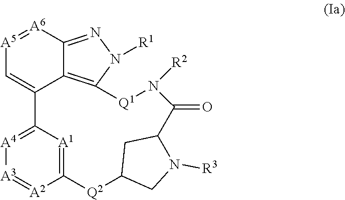

The present invention relates to organic compounds useful for therapy and/or prophylaxis in a mammal, and in particular to antagonist of STING useful for treating autoimmune diseases.

### FIELD OF THE INVENTION

Autoimmune diseases, such as rheumatoid arthritis (RA), systemic lupus erythematosus (SLE), and inflammatory bowel diseases (IBD), refer to a spectrum of conditions where the immune system mistakenly attacks one's own body, leading to unresolved and inappropriately activated inflammation that become pathogenic. Many of the autoimmune diseases are poorly managed by existing treatments that provide only symptomatic relief. Steroid and broad immunosuppressant drugs (e.g. mycophenolate and cyclophosphamide) constitute the stand of care, but are associated with significant treatment-related toxicity. Pathway selective agents such as Adalizumab (anti-TNF antibody, for RA and IBD) occasionally resulting in infection or insufficient tumor surveillance. And Belimumab (anti-BAFF antibody, the only FDA-approved new drug for SLE) shows a slow onset of remission with modest efficacy in the clinic. In addition, the heterogeneity of many autoimmune diseases with no-existing treatment illustrates the difficulty in finding efficacy through the blockade of one immune pathway. Thus, currently available treatments fail to fulfill a greater unmet needs of autoimmune inflammatory diseases with limited remission, severe side effects, opportunistic infection, and poor quality of life with chronic inflammation.

Stimulator of interferon genes (STING) is an endoplasmic reticulum (ER)-located transmembrane protein that is pivotal in mediating the host's innate sensing of pathogen-/damage-associated molecular patterns (PAMPs or DAMPs). In particular, the cyclic-GMP-AMP synthase (cGAS)-STING pathway has emerged as a critical mechanism for coupling cytosolic DNA recognition to the induction of type-I interferon (IFN) and broader immune defense programs. The binding of cGAS to double-stranded DNA (dsDNA) allosterically activates its catalytic site, leading to the production of 2′3′-cyclic GMP-AMP (cGAMP), a secondary messenger molecule that is agonistic to STING. Upon activation, STING translocates from ER to Golgi and recruits TANK-binding kinase 1 (TBK1), which phosphorylates interferon regulatory factor 3 (IRF3) and nuclear factor-kappa B (NF-κB) to initiate the expression of type-I IFN and a myriad of pro-inflammatory cytokines (e.g., IL-6 and TNFα), respectively. Besides 2′3′-cGAMP, STING can be activated by other types of cyclic-di-nucleotides (CDNs), such as c-di-AMP, c-di-GMP, and 3′,3′-cGAMP from bacteria. Following the signal transduction, STING is rapidly degraded to prevent it from constitutive signaling of the inflammatory responses.

While eliciting robust host defense responses, aberrant STING signaling fuels dysregulated immune responses associated with many pathologies. Gain-of-function (GoF) human STING mutations are the root cause of STING-associated vasculopathy with onset in infancy (SAVI), a monogenic disease characterized by the onset of auto-inflammation conditions called type I interferonopathies. Mechanistically, the disease-causing substitutions trigger ligand-independent, constitutive STING activation. Besides, STING is implicated in DNA-driven inflammations, such as Aicardi-Goutieres Syndrome (AGS) and genetic forms of lupus. Unlike SAVI, the STING mediated continuous innate immune activation in AGS is caused by deficiencies in self-DNA clearance and metabolisms due to mutations in endonuclease gene TREX1 and/or DNASE2. Consistently, genetic and pharmacological inhibition of STING ameliorates systemic inflammation and morbidity in the Trex1−/−mouse model. Apart from genetic disorders, robust preclinical and clinical evidence supports a general pathogenic role of STING in a range of inflammation-associated disorders such as SLE and RA. A direct link between the cGAS-STING pathway and SLE was established by observing that PBMC from a subset of SLE patients has elevated cytosolic cGAMP than healthy controls. In addition, membrane vesicles from apoptotic cells in SLE sera have high ISGs-stimulating activities dependent on cGAS-STING. And that disrupting STING signaling ameliorated the development of lupus-like phenotypes in FcγrIIb−/−mice. Furthermore, multiple recent studies associate STING with distinct types of neurodegeneration. Taking Parkinson's disease as an example, missense mutations in PARKIN and PINK resulted in the accumulation of mitochondrial DNA that triggers neuronal inflammation in a cGAS-STING dependent manner. The absence of STING rescued the motor deficit and neuronal cell loss in the mouse disease model. Finally, STING also mediates tumorigenic DNA responses caused by chromosomal instability during cancer metastasis, and that STING-deficiency confers protection against colorectal and skin cancer in the mouse.

### SUMMARY OF THE INVENTION

The present invention relates to novel compounds of formula (I),

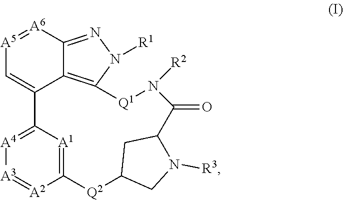

wherein
R¹ is C₁₋₆alkyl;
R² is H, ((C₁₋₆alkylcarbonyl)piperazinyl)C₁₋₆alkyl, (2-oxa-6-azaspiro[3.3]heptanyl)C₁₋₆alkyl, (C₁₋₆alkoxyC₁₋₆alkoxy)C₁₋₆alkyl, (C₁₋₆alkyl)₂aminoC₁₋₆alkyl, (C₁₋₆alkyl-2,5-diazabicyclo[2.2.1]heptanyl)C₁₋₆alkyl, (C₁₋₆alkylpiperazinyl)C₁₋₆alkyl, (C₁₋₆alkylsulfonylpiperazinyl)C₁₋₆alkyl, (C₁₋₆alkyltriazolyl)C₁₋₆alkyl, (pyridinylC₁₋₆alkoxy)C₁₋₆alkyl, 1,2,4-triazolylC₁₋₆alkyl, C₁₋₆alkoxyC₁₋₆alkyl, C₁₋₆alkyl, C₂₋₆alkenyl, C₃₋₇cycloalkylC₁₋₆alkyl, hydroxyC₁₋₆alkyl, imidazolylC₁₋₆alkyl, morpholinylC₁₋₆alkyl, phenylC₁₋₆alkyl, tetrazolylC₁₋₆alkyl or thiazolylC₁₋₆alkyl;
R³ is

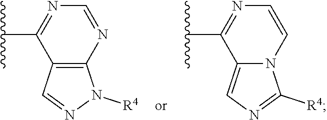

wherein R⁴ is phenyl once, twice or three times substituted by substituents independently selected from halogen and C₁₋₆alkoxy;
Q¹ is C₁₋₆alkylene;
Q² is NH or O;
A¹ is CH or N;
A² is CH or N;
A³ is CR⁵ or N, wherein R⁵ is H, C₁₋₆alkoxy or C₁₋₆alkyl;
A⁴ is CH or N;
A⁵ is CR⁶, wherein R⁶ is H or halogen;
A⁶ is CR⁷, wherein R⁷ is H or halogen;
provided that no more than two of A¹, A², A³ and A⁴ are N simultaneously; or a pharmaceutically acceptable salt thereof.

Another object of the present invention is related to novel compounds of formula (I) or (Ia). Their manufacture, medicaments based on a compound in accordance with the invention and their production as well as the use of compounds of formula (I) or (Ia) as STING antagonist, and for the treatment or prophylaxis of autoimmune diseases, inflammatory diseases, neurological disorders diseases, metabolic diseases, cardiovascular diseases, or selective types of cancers where overexpression or activation of STING is implicated. The compounds of formula (I) or (Ia) show superior STING antagonism activity. In addition, the compounds of formula (I) or (Ia) also show good cytotoxicity, phototoxicity, solubility, hPBMC, metabolic stability, hERG and SDPK profiles, as well as low CYP inhibition.

### DETAILED DESCRIPTION OF THE INVENTION

### Definitions

The term “C₁₋₆alkyl” denotes a saturated, linear or branched chain alkyl group containing 1 to 6, particularly 1 to 4 carbon atoms, for example methyl, ethyl, n-propyl, isopropyl, n-butyl, isobutyl, tert-butyl and the like. Particular “C₁₋₆alkyl” groups are methyl, ethyl and n-propyl.

The term “C₁₋₆alkylene” denotes a linear or branched saturated divalent hydrocarbon group of 1 to 6 carbon atoms or a divalent branched saturated divalent hydrocarbon group of 3 to 6 carbon atoms. Examples of C₁₋₆alkylene groups include methylene, ethylene, propylene, 2-methylpropylene, butylene, 2-ethylbutylene, pentylene, hexylene.

The term “C₁₋₆alkoxy” denotes C₁₋₆alkyl-O—.

The term “C₂₋₆alkenyl” denotes a monovalent linear or branched hydrocarbon group of 2 to 6 carbon atoms with at least one double bond. In particular embodiments, alkenyl has 2 to 4 carbon atoms with at least one double bond. Examples of C₂₋₆alkenyl include ethenyl (or vinyl), propenyl, allyl, prop-2-enyl, isopropenyl, n-butenyl, and iso-butenyl.

The term “C₃₋₇cycloalkyl” denotes a monovalent saturated monocyclic or bicyclic hydrocarbon group of 3 to 7 ring carbon atoms. Bicyclic means consisting of two saturated carbocycles having one or more carbon atoms in common. Examples for monocyclic cycloalkyl are cyclopropyl, cyclobutyl, cyclopentyl, cyclohexyl or cycloheptyl. Examples for bicyclic cycloalkyl are bicyclo[1.1.0]butyl, bicyclo[2.2.1]heptanyl, bicyclo[1.1.1]pentanyl, or bicyclo[2.2.2]octanyl.

The term “PG” denotes protecting groups.

The term “pharmaceutically acceptable salts” denotes salts which are not biologically or otherwise undesirable. Pharmaceutically acceptable salts include both acid and base addition salts.

The term “pharmaceutically acceptable acid addition salt” denotes those pharmaceutically acceptable salts formed with inorganic acids such as hydrochloric acid, hydrobromic acid, sulfuric acid, nitric acid, carbonic acid, phosphoric acid, and organic acids selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, and salicyclic acid.

The term “pharmaceutically acceptable base addition salt” denotes those pharmaceutically acceptable salts formed with an organic or inorganic base. Examples of acceptable inorganic bases include sodium, potassium, ammonium, calcium, magnesium, iron, zinc, copper, manganese, and aluminum salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, and polyamine resins.

The term “A pharmaceutically active metabolite” denotes a pharmacologically active product produced through metabolism in the body of a specified compound or salt thereof. After entry into the body, most drugs are substrates for chemical reactions that may change their physical properties and biologic effects. These metabolic conversions, which usually affect the polarity of the compounds of the invention, alter the way in which drugs are distributed in and excreted from the body. However, in some cases, metabolism of a drug is required for therapeutic effect.

The term “therapeutically effective amount” denotes an amount of a compound or molecule of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the compound, the disease state being treated, the severity of the disease treated, the age and relative health of the subject, the route and form of administration, the judgement of the attending medical or veterinary practitioner, and other factors.

The term “pharmaceutical composition” denotes a mixture or solution comprising a therapeutically effective amount of an active pharmaceutical ingredient together with pharmaceutically acceptable excipients to be administered to a mammal, e.g., a human in need thereof.

The terms “pharmaceutically acceptable excipient”, “pharmaceutically acceptable carrier” and “therapeutically inert excipient” can be used interchangeably and denote any pharmaceutically acceptable ingredient in a pharmaceutical composition having no therapeutic activity and being non-toxic to the subject administered, such as disintegrators, binders, fillers, solvents, buffers, tonicity agents, stabilizers, antioxidants, surfactants, carriers, diluents or lubricants used in formulating pharmaceutical products.

### Antagonist of Sting

The present invention relates to (i) a compound of formula (I),

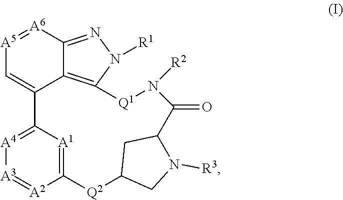

wherein
R¹ is C₁₋₆alkyl;
R² is H, ((C₁₋₆alkylcarbonyl)piperazinyl)C₁₋₆alkyl, (2-oxa-6-azaspiro[3.3]heptanyl)C₁₋₆alkyl, (C₁₋₆alkoxyC₁₋₆alkoxy)C₁₋₆alkyl, (C₁₋₆alkyl)₂aminoC₁₋₆alkyl, (C₁₋₆alkyl-2,5-diazabicyclo[2.2.1]heptanyl)C₁₋₆alkyl, (C₁₋₆alkylpiperazinyl)C₁₋₆alkyl, (C₁₋₆alkylsulfonylpiperazinyl)C₁₋₆alkyl, (C₁₋₆alkyltriazolyl)C₁₋₆alkyl, (pyridinylC₁₋₆alkoxy)C₁₋₆alkyl, 1,2,4-triazolylC₁₋₆alkyl, C₁₋₆alkoxyC₁₋₆alkyl, C₁₋₆alkyl, C₂₋₆alkenyl, C₃₋₇cycloalkylC₁₋₆alkyl, hydroxyC₁₋₆alkyl, imidazolylC₁₋₆alkyl, morpholinylC₁₋₆alkyl, phenylC₁₋₆alkyl, tetrazolylC₁₋₆alkyl or thiazolylC₁₋₆alkyl;

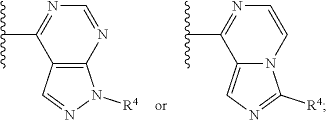

wherein R⁴ is phenyl once, twice or three times substituted by substituents independently selected from halogen and C₁₋₆alkoxy;
Q¹ is C₁₋₆alkylene;
Q² is NH or O;
A¹ is CH or N;
A² is CH or N;
A³ is CR⁵ or N, wherein R⁵ is H, C₁₋₆alkoxy or C₁₋₆alkyl;
A⁴ is CH or N;
A⁵ is CR⁶, wherein R⁶ is H or halogen;
A⁶ is CR⁷, wherein R⁷ is H or halogen;
provided that no more than two of A¹, A², A³ and A⁴ are N simultaneously;
or a pharmaceutically acceptable salt thereof.

Another embodiment of present invention is (ii) a compound of formula (Ia),

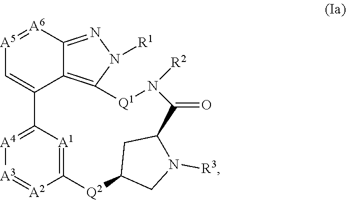

wherein
R¹ is C₁₋₆alkyl;
R² is H, ((C₁₋₆alkylcarbonyl)piperazinyl)C₁₋₆alkyl, (2-oxa-6-azaspiro[3.3]heptanyl)C₁₋₆alkyl, (C₁₋₆alkoxyC₁₋₆alkoxy)C₁₋₆alkyl, (C₁₋₆alkyl)₂aminoC₁₋₆alkyl, (C₁₋₆alkyl-2,5-diazabicyclo[2.2.1]heptanyl)C₁₋₆alkyl, (C₁₋₆alkylpiperazinyl)C₁₋₆alkyl, (C₁₋₆alkylsulfonylpiperazinyl)C₁₋₆alkyl, (C₁₋₆alkyltriazolyl)C₁₋₆alkyl, (pyridinylC₁₋₆alkoxy)C₁₋₆alkyl, 1,2,4-triazolylC₁₋₆alkyl, C₁₋₆alkoxyC₁₋₆alkyl, C₁₋₆alkyl, C₂₋₆alkenyl, C₃₋₇cycloalkylC₁₋₆alkyl, hydroxyC₁₋₆alkyl, imidazolylC₁₋₆alkyl, morpholinylC₁₋₆alkyl, phenylC₁₋₆alkyl, tetrazolylC₁₋₆alkyl or thiazolylC₁₋₆alkyl;
R³ is

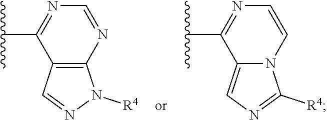

wherein R⁴ is phenyl once, twice or three times substituted by substituents independently selected from halogen and C₁₋₆alkoxy;
Q¹ is C₁₋₆alkylene;
Q² is NH or O;
A¹ is CH or N;
A² is CH or N;
A³ is CR⁵ or N, wherein R⁵ is H, C₁₋₆alkoxy or C₁₋₆alkyl;
A⁴ is CH or N;
A⁵ is CR⁶, wherein R⁶ is H or halogen;
A⁶ is CR⁷, wherein R⁷ is H or halogen;
provided that no more than two of A¹, A², A³ and A⁴ are N simultaneously;
or a pharmaceutically acceptable salt thereof.

A further embodiment of present invention is (iii) a compound of formula (I) or (Ia) according to (i) or (ii), or a pharmaceutically acceptable salt thereof, wherein R¹ is methyl.

A further embodiment of present invention is (iv) a compound of formula (I) or (Ia) according to any one of (i) to (iii), or a pharmaceutically acceptable salt thereof, wherein R² is C₁₋₆alkoxyC₁₋₆alkyl, C₁₋₆alkyl, morpholinylC₁₋₆alkyl or phenylC₁₋₆alkyl.

A further embodiment of present invention is (v) a compound of formula (I) or (Ia), according to any one of (i) to (iv), or a pharmaceutically acceptable salt thereof, wherein R² is 3-methoxypropyl, 3-morpholinylpropyl, 3-phenylpropyl or methyl.

A further embodiment of present invention is (vi) a compound of formula (I) or (Ia) according to any one of (i) to (v), wherein R³ is

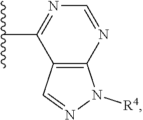

wherein R⁴ is phenyl twice substituted by substituents independently selected from halogen and C₁₋₆alkoxy.

A further embodiment of present invention is (vii) a compound of formula (I) or (Ia), or a pharmaceutically acceptable salt thereof, according to any one of (i) to (vi), wherein R³ is

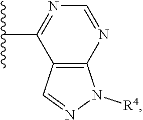

wherein R⁴ is 2,4-difluorophenyl or 4-fluoro-2-methoxy-phenyl.

A further embodiment of present invention is (viii) a compound of formula (I) or (Ia), or a pharmaceutically acceptable salt thereof, according to any one of (i) to (vii), wherein Q¹ is propylene.

A further embodiment of present invention is (ix) a compound of formula (I) or (Ia), or a pharmaceutically acceptable salt thereof, according to any one of (i) to (viii), wherein A³ is CR⁵ or N, wherein R⁵ is H or C₁₋₆alkoxy.

A further embodiment of present invention is (x) a compound of formula (I) or (Ta), or a pharmaceutically acceptable salt thereof, according to any one of (i) to (ix), wherein A³ is CR⁵ or N, wherein R⁵ is H or methoxy.

A further embodiment of present invention is (xi) a compound of formula (I) or (Ta), or a pharmaceutically acceptable salt thereof, according to any one of (i) to (x), wherein A⁵ is CH.

A further embodiment of present invention is (xii) a compound of formula (I) or (Ta), or a pharmaceutically acceptable salt thereof, according to any one of (i) to (xi), wherein A⁶ is CH.

A further embodiment of present invention is (xiii) a compound of formula (I) or (Ta), according to any one of (i) or (ii), wherein
R¹ is C₁₋₆alkyl;
R² is C₁₋₆alkoxyC₁₋₆alkyl, C₁₋₆alkyl, morpholinylC₁₋₆alkyl or phenylC₁₋₆alkyl;
R³ is

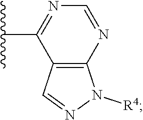

wherein R⁴ is phenyl twice substituted by substituents independently selected from halogen and C₁₋₆alkoxy;
Q¹ is C₁₋₆alkylene;
Q² is NH or O;
A¹ is CH or N;
A² is CH or N;
A³ is CR⁵ or N, wherein R⁵ is H or C₁₋₆alkoxy;
A⁴ is CH or N;
A⁵ is CH;
A⁶ is CH;
provided that no more than two of A¹, A², A³ and A⁴ are N simultaneously;
   or a pharmaceutically acceptable salt thereof.

A further embodiment of present invention is (xiv) a compound of formula (I) or (Ia), according to (xiii), wherein
R¹ is methyl;
R² is 3-methoxypropyl, 3-morpholinylpropyl, 3-phenylpropyl or methyl;
R³ is

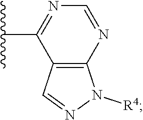

wherein R⁴ is 2,4-difluorophenyl or 4-fluoro-2-methoxy-phenyl;
Q¹ is propylene;
Q² is NH or O;
A¹ is CH or N;
A² is CH or N;
A³ is CR⁵ or N, wherein R⁵ is H or methoxy;
A⁴ is CH or N;
A⁵ is CH;
A⁶ is CH;
provided that no more than two of A¹, A², A³ and A⁴ are N simultaneously;
   or a pharmaceutically acceptable salt thereof.

Another embodiment of present invention (xv) is a compound of formula (I) or (Ia) selected from the following:
(8S,11S)-10-[1-(2,4-difluorophenyl)pyrazolo[3,4-d]pyrimidin-4-yl]-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-13,18-dimethyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-13,18-dimethyl-7,10,13,18,19,26-hexazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-13,18-dimethyl-5,7,10,13,18,19,26-heptazapentacyclo[15.6.1.1^{2,6}.1^{8,11}.0^{20,24}]hexacosa-1(23),2,4,6(26),17(24),19,21-heptaen-12-one;
(8S,11S)-10-[3-(2,4-difluorophenyl)imidazo[1,5-a]pyrazin-8-yl]-13,18-dimethyl-5,7,10,13,18,19,26-heptazapentacyclo[15.6.1.1^{2,6}.1^{8,11}.0^{20,24}]hexacosa-1(23),2,4,6(26),17(24),19,21-heptaen-12-one;
(8S,11S)-10-[11-(4-fluoro-2-methoxy-phenyl)pyrazolo[3,4-d]pyrimidin-4-yl]-13,18-dimethyl-5,7,10,13,18,19,26-heptazapentacyclo[15.6.1.1^{2,6}.1^{8,11}.0^{20,24}]hexacosa-1(23),2,4,6(26),17(24),19,21-heptaen-12-one;
(8S,11S)-10-[11-(2,4-difluorophenyl)pyrazolo[3,4-d]pyrimidin-4-yl]-13,18-dimethyl-4,7,10,13,18,19,26-heptazapentacyclo[15.6.1.1^{2,6}.1^{8,11}.0^{20,24}]hexacosa-1(23),2,4,6(26),17(24),19,21-heptaen-12-one;
(8S,11S)-10-[11-(2,4-difluorophenyl)pyrazolo[3,4-d]pyrimidin-4-yl]-13,18-dimethyl-3,7,10,13,18,19,26-heptazapentacyclo[15.6.1.1^{2,6}.1^{8,11}.0^{20,24}]hexacosa-1(23),2,4,6(26),17(24),19,21-heptaen-12-one;
(8S,11S)-10-[11-(2,4-difluorophenyl)pyrazolo[3,4-d]pyrimidin-4-yl]-13,18-dimethyl-7-oxa-10,13,18,19,26-pentazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-18-methyl-7-oxa-3,10,13,18,19-pentazapentacyclo[15.6.1.1^{2,6}.1^{8,11}.0^{20,24}]hexacosa-1(23),2(26),3,5,17(24),19,21-heptaen-12-one;
(8S,11S)-10-[11-(2,4-difluorophenyl)pyrazolo[3,4-d]pyrimidin-4-yl]-13,18-dimethyl-7-oxa-5,10,13,18,19,26-hexazapentacyclo[15.6.1.1^{2,6}.1^{8,11}.0^{20,24}]hexacosa-1(23),2(26),3,5,17(24),19,21-heptaen-12-one;
(8S,11S)-22-fluoro-10-[1-(4-fluoro-2-methoxy-phenyl)pyrazolo[3,4-d]pyrimidin-4-yl]-13,18-dimethyl-7-oxa-5,10,13,18,19,26-hexazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-4-methoxy-13,18-dimethyl-7,10,13,18,19,26-hexazapentacyclo[15.6.1.1^{2,6}.1^{8,11}.0^{20,24}]hexacosa-1(23),2,4,6(26),17(24),19,21-heptaen-12-one;
(8S,11S)-10-[1-(4-fluoro-2-methoxy-phenyl)pyrazolo[3,4-d]pyrimidin-4-yl]-4-methoxy-13,18-dimethyl-7,10,13,18,19,26-hexazapentacyclo[15.6.1.1^{2,6}.1^{8,11}.0^{20,24}]hexacosa-1(23),2,4,6(26),17(24),19,21-heptaen-12-one;
(8S,11S)-13-(cyclopropylmethyl)-10-[1-(2,4-difluorophenyl)pyrazolo[3,4-d]pyrimidin-4-yl]-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-18-methyl-13-[(1-methyl-1,2,4-triazol-3-yl)methyl]-7-oxa-10,13,18,19 tetrazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-18-methyl-13-(thiazol-4-ylmethyl)-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-13-(cyclopropylmethyl)-10-[1-(2,4-difluorophenyl)pyrazolo[3,4-d]pyrimidin-4-yl]-18-methyl-7-oxa-3,5,10,13,18,19-hexazapentacyclo[15.6.1.1^{2,6}.1^{8,11}.0^{20,24}]hexacosa-1(23),2(26),3,5,17(24),19,21-heptaen-12-one;
(8S,11S)-13-(cyclopropylmethyl)-10-[1-(2,4-difluorophenyl)pyrazolo[3,4-d]pyrimidin-4-yl]-18-methyl-7-oxa-5,10,13,18,19,26-hexazapentacyclo[15.6.1.1^{2,6}.1^{8,11}.0^{20,24}]hexacosa-1(23),2(26),3,5,17(24),19,21-heptaen-12-one;
(8S,11S)-13-allyl-10-[1-(2,4-difluorophenyl)pyrazolo[3,4-d]pyrimidin-4-yl]-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-13-(2-methoxyethyl)-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-13-(2-hydroxyethyl)-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-18-methyl-13-(2-morpholinoethyl)-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-13-[2-(dimethylamino)ethyl]-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-22-fluoro-18-methyl-13-(2-morpholinoethyl)-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-13-(3-hydroxypropyl)-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-13-(3-methoxypropyl)-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-18-methyl-13-[2-(4-pyridylmethoxy)ethyl]-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-18-methyl-13-(3-phenylpropyl)-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-18-methyl-13-(3-morpholinopropyl)-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-18-methyl-13-[3-(2-oxa-6-azaspiro[3.3]heptan-6-yl)propyl]-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-18-methyl-13-[3-(4-methylpiperazin-1-yl)propyl]-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-13-[3-(4-acetylpiperazin-1-yl)propyl]-10-[1-(2,4-difluorophenyl)pyrazolo[3,4-d]pyrimidin-4-yl]-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-18-methyl-13-[3-(5-methyl-2,5-diazabicyclo[2.2.1]heptan-2-yl)propyl]-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-18-methyl-13-[3-(4-methylsulfonylpiperazin-1-yl)propyl]-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-18-methyl-13-[3-(tetrazol-1-yl)propyl]-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-18-methyl-13-[3-(1,2,4-triazol-1-yl)propyl]-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-13-(3-imidazol-1-ylpropyl)-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-13-[3-(2-methoxyethoxy)propyl]-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-10-[11-(2,4-difluorophenyl)pyrazolo[3,4-d]pyrimidin-4-yl]-13-(3-hydroxy-3-methyl-butyl)-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-18-methyl-13-(3-phenylpropyl)-7-oxa-10,13,18,19,26-pentazapentacyclo[15.6.1.1^{2,6}.1^{8,11}.0^{20,24}]hexacosa-1(23),2(26),3,5,17(24),19,21-heptaen-12-one;
(8S,11S)-10-[11-(2,4-difluorophenyl)pyrazolo[3,4-d]pyrimidin-4-yl]-13-(3-hydroxypropyl)-18-methyl-7,10,13,18,19,26-hexazapentacyclo[15.6.1.1^{2,6}.1^{8,11}.0^{20,24}]hexacosa-1(23),2(26),3,5,17(24),19,21-heptaen-12-one;
(8S,11S)-10-[1-(4-fluoro-2-methoxy-phenyl)pyrazolo[3,4-d]pyrimidin-4-yl]-18-methyl-13-(3-morpholinopropyl)-7,10,13,18,19,26-hexazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-21-fluoro-18-methyl-13-(3-morpholinopropyl)-7,10,13,18,19,26-hexazapentacyclo[15.6.1.1^{2,6}.1^{8,11}.0^{20,24}]hexacosa-1(23),2(26),3,5,17(24),19,21-heptaen-12-one;
(8S,11S)-21-fluoro-10-[1-(4-fluoro-2-methoxy-phenyl)pyrazolo[3,4-d]pyrimidin-4-yl]-18-methyl-13-(3-morpholinopropyl)-7,10,13,18,19,26-hexazapentacyclo[15.6.1.1^{2,6}.1^{8,11}.0^{20,24}]hexacosa-1(23),2(26),3,5,17(24),19,21-heptaen-12-one;
(8S,11S)-10-[11-(4-fluoro-2-methoxy-phenyl)pyrazolo[3,4-d]pyrimidin-4-yl]-4,18-dimethyl-13-(3-morpholinopropyl)-7,10,13,18,19,26-hexazapentacyclo[15.6.1.1^{2,6}.1^{8,11}.0^{20,24}]hexacosa-1(23),2(26),3,5,17(24),19,21-heptaen-12-one; and
(8S,11S)-10-[1-(4-fluoro-2-methoxy-phenyl)pyrazolo[3,4-d]pyrimidin-4-yl]-18-methyl-13-(3-morpholinopropyl)-5,7,10,13,18,19,26-heptazapentacyclo[15.6.1.1^{2,6}.1^{8,11}.0^{20,24}]hexacosa-1(23),2(26),3,5,17(24),19,21-heptaen-12-one;

or a pharmaceutically acceptable salt thereof.

Another embodiment of present invention (xvi) is a process for the preparation of a compound according to any one of (i) to (xv) comprising the following step:
a) the formation of compound of formula (I) via nucleophilic substitution between compound of formula (VII),

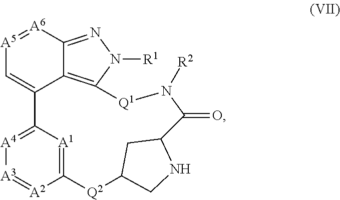

and R³X, in the presence of a base;
   wherein the base is DIEA;
   R^{a} is H or PG, wherein PG is Boc, Cbz, PMB or phthaloyl; X is halogen.
   R¹ to R³, Q¹, Q², A¹ to A⁶ are as defined as in any one of (i) to (xiv).

Another embodiment of present invention (xvii) is related to a compound or pharmaceutically acceptable salt according to any one of (i) to (xv) for use as therapeutically active substance.

Another embodiment of present invention (xviii) is related to a pharmaceutical composition comprising a compound in accordance with any one of (i) to (xv) and a pharmaceutically acceptable excipient.

Another embodiment of present invention (xix) is related to the use of a compound according to any one of (i) to (xv) for the treatment or prophylaxis of autoimmune diseases, inflammatory diseases, neurological disorders diseases, metabolic diseases, cardiovascular diseases, ocular diseases, or selective types of cancers where overexpression or activation of STING is implicated.

Another embodiment of present invention (xx) is related to the use of a compound according to any one of (i) to (xv) for the treatment to subjects suffered from an inteferonopathy or auto-inflammatory diseases in which the STING activation are the root-cause of disease pathologies.

Another embodiment of present invention (xxi) is related to a compound or pharmaceutically acceptable salt according to any one of (i) to (xv) for the treatment or prophylaxis of autoimmune diseases, inflammatory diseases, neurological disorders diseases, metabolic diseases, cardiovascular diseases, ocular diseases, or selective types of cancers where overexpression or activation of STING is implicated.

Another embodiment of present invention (xxii) is related to the use of a compound according to any one of (i) to (xv) for the inhibition of STING.

Another embodiment of present invention (xxiii) is related to the use of a compound according to any one of (i) to (xv) for the preparation of a medicament for the inhibition of STING.

Another embodiment of present invention (xxiv) is related to a compound or pharmaceutically acceptable salt according to any one of (i) to (xv), when manufactured according to a process of (xvi).

Another embodiment of present invention (xxv) is related to a method for the treatment or prophylaxis of autoimmune diseases, which method comprises administering a therapeutically effective amount of a compound as defined in any one of (i) to (xv).

### Pharmaceutical Compositions and Administration

Another embodiment provides pharmaceutical compositions or medicaments containing the compounds of the invention and a therapeutically inert carrier, diluent or excipient, as well as methods of using the compounds of the invention to prepare such compositions and medicaments. In one example, compounds of formula (I) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula (I) is formulated in an acetate buffer, at pH 5. In another embodiment, the compounds of formula (I) are sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “effective amount” of the compound to be administered will be governed by such considerations, and is the minimum amount necessary to inhibit STING interaction with IRF3, NF-kB, NLRP3 etc., for blocking downstream type I IFN and pro-inflammatory cytokine (e.g. IL-6, TNFα, ISGs) production, cellular responses/conditions (e.g. autophagy, apoptosis, cell senescence). For example, such amount may be below the amount that is toxic to normal cells, or the mammal as a whole.

In one example, the pharmaceutically effective amount of the compound of the invention administered parenterally per dose will be in the range of about 0.1 to 1000 mg/kg, alternatively about 0.1 to 1000 mg/kg of patient body weight per day, with the typical initial range of compound used being 0.1 to 1000 mg/kg/day. In another embodiment, oral unit dosage forms, such as tablets and capsules, preferably contain from about 0.1 to about 1000 mg of the compound of the invention.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

An example of a suitable oral dosage form is a tablet containing about 0.1 to 1000 mg of the compound of the invention compounded with about 0.1 to 1000 mg anhydrous lactose, about 0.1 to 1000 mg sodium croscarmellose, about 0.1 to 1000 mg polyvinylpyrrolidone (PVP) K30, and about 0.1 to 1000 mg magnesium stearate. The powdered ingredients are first mixed together and then mixed with a solution of the PVP. The resulting composition can be dried, granulated, mixed with the magnesium stearate and compressed to tablet form using conventional equipment. An example of an aerosol formulation can be prepared by dissolving the compound, for example 0.1 to 1000 mg, of the invention in a suitable buffer solution, e.g. a phosphate buffer, adding a tonicifier, e.g. a salt such sodium chloride, if desired. The solution may be filtered, e.g., using a 0.2 micron filter, to remove impurities and contaminants.

An embodiment, therefore, includes a pharmaceutical composition comprising a compound of formula (I), or a stereoisomer or pharmaceutically acceptable salt thereof. In a further embodiment includes a pharmaceutical composition comprising a compound of formula (I), or a stereoisomer or pharmaceutically acceptable salt thereof, together with a pharmaceutically acceptable carrier or excipient.

Another embodiment includes a pharmaceutical composition comprising a compound of formula (I) for use in the treatment of interferonopathies, autoimmune and inflammatory diseases. Another embodiment includes a pharmaceutical composition comprising a compound of formula (I) for use in the treatment of infection or selective type of cancer.

The following composition A and B illustrate typical compositions of the present invention, but serve merely as representative thereof.

### Composition A

A compound of the present invention can be used in a manner known per se as the active ingredient for the production of tablets of the following composition:

| | Per tablet | |
|---|---|---|
| Active ingredient | 200 | mg |
| Microcrystalline cellulose | 155 | mg |
| Corn starch | 25 | mg |
| Talc | 25 | mg |
| Hydroxypropylmethylcellulose | 20 | mg |
| | 425 | mg |

### Composition B

A compound of the present invention can be used in a manner known per se as the active ingredient for the production of capsules of the following composition:

| | Per capsule | |
|---|---|---|
| Active ingredient | 100.0 | mg |
| Corn starch | 20.0 | mg |
| Lactose | 95.0 | mg |
| Talc | 4.5 | mg |
| Magnesium stearate | 0.5 | mg |
| | 220.0 | mg |

### Indications and Methods of Treatment

The compounds of the invention inhibit STING, including its signaling and activities. Accordingly, the compounds of the invention are useful for infection, inflammation, autoimmune, degenerative diseases and cancer therapy.

In some embodiments, compounds of the invention are useful for the treatment or prophylaxis of autoimmune diseases.

In some embodiments, compounds of the invention are useful for the treatment or prophylaxis of inflammatory diseases.

In some embodiments, compounds of the invention are useful for the treatment or prophylaxis of neurological disorders diseases.

In some embodiments, compounds of the invention are useful for the treatment or prophylaxis of cardiovascular diseases.

In some embodiments, compounds of the invention are useful for the treatment or prophylaxis of ocular diseases.

In some embodiments, compounds of the invention are useful for the treatment or prophylaxis of selective types of cancers where overexpression or activation of STING is implicated.

Alternatively, compounds of the invention are useful for the treatment of subjects suffered from an inteferonopathy or auto-inflammatory diseases in which the STING activation are the root-cause of disease pathologies. More broadly, the compounds can be used for the treatment of all pathological cellular processes which are STING dependent.

Another embodiment includes a method of treating or preventing cancer in a mammal in need of such treatment, wherein the method comprises administering to said mammal a therapeutically effective amount of a compound of formula (I), a stereoisomer, tautomer, prodrug or pharmaceutically acceptable salt thereof.

### Synthesis

The compounds of the present invention can be prepared by any conventional means. Suitable processes for synthesizing these compounds as well as their starting materials are provided in the schemes below and in the examples. All substituents, in particular, R¹ to R⁴, Q¹, Q², A¹ to A⁶ are as defined above unless otherwise indicated. Furthermore, and unless explicitly otherwise stated, all reactions, reaction conditions, abbreviations and symbols have the meanings well known to a person of ordinary skill in organic chemistry.

General synthetic routes for preparing the compound of the invention are shown in following schemes.

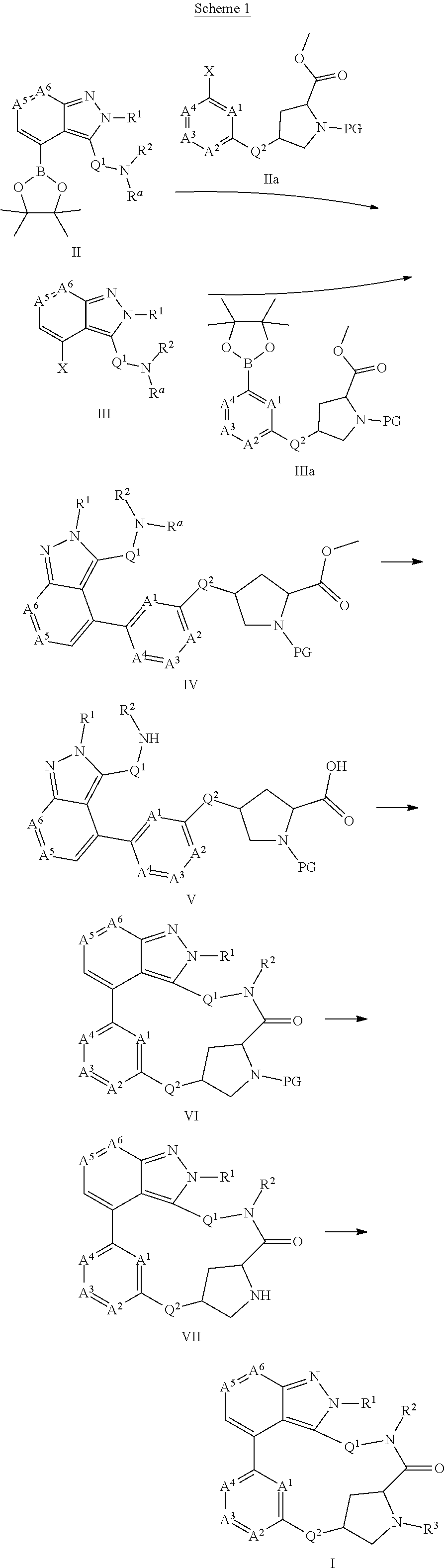

Wherein R^{a} is H or PG, wherein PG is Boc, Cbz, PMB or phthaloyl; X is halogen.

As depicted in Scheme 1, the synthesis of compounds of the present invention started from boronic ester compound of formula (II) or halide (III). Suzuki coupling between compound of formula (II) and compound of formula (IIa) with a catalyst, such as Pd(dppf)Cl₂, and a base, such as K₂CO₃ provides compound of formula (IV), which can also be obtained via the Suzuki coupling between boronic ester compound of formula (IIIa) and halide (III). Compound of formula (IV) is hydrolyzed in the presence of LiOH directly or followed by appropriate deprotection to give compound of (V) (Boc deprotection: HCl in dioxane or TFA in DCM; PMB deprotection: Pd/C under H₂; phthaloyl deprotection: hydrazine in MeOH). Compound of formula (V) can be cyclized to give compound of formula (VI) in the presence of a condensation reagent, such as HATU, and a base, such as DIPEA. The following Boc deprotection in acidic condition (HCl in dioxane or TFA in DCM) or Cbz deprotection by catalytic hydrogenation (Pd/C or Pd(OH)₂/C under H₂) or in acidic condition (TFA) to give compound of formula (VII). The final compounds of formula (I) can be obtained by the reaction between compound of formula (VII) and R₃X via nucleophilic substitution in the presence of a base, such as DIEA.

Compounds of this invention can be obtained as mixtures of diastereomers or enantiomers, which can be separated by methods well known in the art, e.g. (chiral) HPLC or SFC.

This invention also relates to a process for the preparation of a compound of formula (I) comprising the following step:
b) the formation of compound of formula (I) via nucleophilic substitution between compound of formula (VII),

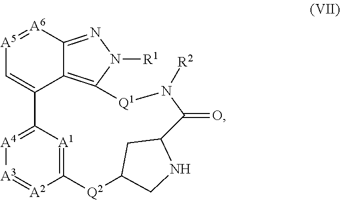

and R³X, in the presence of a base;
wherein the base can be, for example, DIEA;
R^{a} is H or PG, wherein PG is Boc, Cbz, PMB or phthaloyl; X is halogen.

A compound of formula (I) when manufactured according to the above process is also an object of the invention.

### EXAMPLES

The invention will be more fully understood by reference to the following examples. They should not, however, be construed as limiting the scope of the invention.

### Abbreviations

The invention will be more fully understood by reference to the following examples. They should not, however, be construed as limiting the scope of the invention.

Abbreviations used herein are as follows:
ACN: acetonitrile
Boc₂O: di-tert butyl dicarbonate
cataCXium A-Pd-G2: chloro[(di(1-adamantyl)-N-butylphosphine)-2-(2-aminobiphenyl)]palladium(II)
DCM: dichloromethane
DIPEA: N,N-Diisopropylethylamine
DIAD: diisopropyl azodicarboxylate
DMF: N,N-Dimethylformamide
DMSO: dimethyl sulfoxide
EtOAc: ethyl acetate
FA: formic acid
HATU: 1-[bis(dimethylamino)methylene]-1H-1,2,3-triazolo[4,5-b]pyridinium 3-oxid hexafluorophosphate
h(s) or hr(s): hour (s)
hPBMC: human peripheral blood mononuclear cells
IC₅₀: half inhibition concentration
IPA: isopropanol
LCMS: liquid chromatography-mass spectrometry
LDA: lithium diisopropylamide
min(s): minute(s)
MS: mass spectrometry
NMP: N-methylpyrrolidone
prep-HPLC: preparative high performance liquid chromatography
prep-TLC: preparative thin layer chromatography
PPh₃: triphenylphosphine
Pd₂(dba)₃: tris(dibenzylideneacetone)dipalladium(0) Pd(dppf)Cl₂·DCM: 1,1′-bis(diphenylphosphino)ferrocene-palladium(II) dichloromethane complex
Rf: retention factor
RT: retention time
SFC: supercritical fluid chromatography
TEA: trimethylamine
TFA: trifluoroacetic acid
THF: tetrahydrofuran
TLC: thin layer chromatography
XantPhos: 4,5-bis(diphenylphosphino)-9,9-dimethylxanthene
XPhos: 2-dicyclohexylphosphino-2′,4′,6′-triisopropylbiphenyl
v/v volume ratio

### General Experimental Conditions

Intermediates and final compounds were purified by flash column chromatography using one of the following instruments: i) Biotage SP1 system and the Quad 12/25 Cartridge module. ii) ISCO combi-flash column instrument. Silica gel brand and pore size: i) KP-SIL 60 Å, particle size: 40-60 μm; ii) CAS registry NO: Silica Gel: 63231-67-4, particle size: 47-60 micron silica gel; iii) ZCX from Qingdao Haiyang Chemical Co., Ltd, pore: 200-300 or 300-400.

Intermediates and final compounds were purified by preparative HPLC on reversed phase column using XBridge™ Prep-C18 (5 μm, OBD™ 30×100 mm) column, SunFire™ Prep-C18 (5 μm, OBD™ 30×100 mm) column, Phenomenex Synergi-C₁₈ (10 μm, 25×150 mm) or Phenomenex Gemini-C18 (10 μm, 25×150 mm). Waters AutoP purification System (Sample Manager 2767, Pump 2525, Detector: Micromass ZQ and UV 2487, solvent system: acetonitrile and 0.1% ammonium hydroxide in water; acetonitrile and 0.1% FA in water or acetonitrile and 0.1% TFA in water). Or Gilson-281 purification System (Pump 322, Detector: UV 156, solvent system: acetonitrile and 0.05% ammonium hydroxide in water; acetonitrile and 0.225% FA in water; acetonitrile and 0.05% HCl in water; acetonitrile and 0.075% TFA in water; or acetonitrile and water).

For SFC chiral separation, intermediates were separated by chiral column (Daicel chiralpak IC, 5 μm, 30×250 mm), AS (10 μm, 30×250 mm) or AD (10 μm, 30×250 mm) using Mettler Toledo Multigram III system SFC, Waters 80Q preparative SFC or Thar 80 preparative SFC, solvent system: CO₂ and IPA (0.5% TEA in IPA) or CO₂ and MeOH (0.1% NH₃·H₂O in MeOH), back pressure 100 bar, detection UV@ 254 or 220 nm.

LC/MS spectra of compounds were obtained using a LC/MS (Waters™ Alliance 2795-Micromass ZQ, Shimadzu Alliance 2020-Micromass ZQ or Agilent Alliance 6110-Micromass ZQ), LC/MS conditions were as follows (running time 3 or 1.5 mins):
Acidic condition I: A: 0.1% TFA in H₂O; B: 0.1% TFA in acetonitrile;
Acidic condition II: A: 0.0375% TFA in H₂O; B: 0.01875% TFA in acetonitrile;
Basic condition I: A: 0.1% NH₃·H₂O in H₂O; B: acetonitrile;
Basic condition II: A: 0.025% NH₃·H₂O in H₂O; B: acetonitrile;
Neutral condition: A: H₂O; B: acetonitrile.

Mass spectra (MS): generally only ions which indicate the parent mass are reported, and unless otherwise stated the mass ion quoted is the positive mass ion (MH)⁺.

NMR Spectra were obtained using Bruker Avance 400 MHz, 500 MHz.

The microwave assisted reactions were carried out in a Biotage Initiator Sixty microwave synthesizer. All reactions involving air-sensitive reagents were performed under an argon or nitrogen atmosphere. Reagents were used as received from commercial suppliers without further purification unless otherwise noted.

### PREPARATIVE EXAMPLES

The following examples are intended to illustrate the meaning of the present invention but should by no means represent a limitation within the meaning of the present invention:

### Intermediate A1

### 2-[3-[2-Methyl-4-(4,4,5,5-tetramethyl-1,3,2-dioxaborolan-2-yl)indazol-3-yl]propyl]isoindoline-1,3-dione

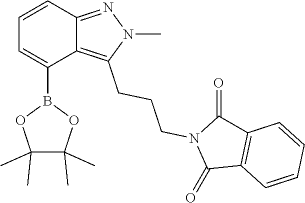 [SMILES: Cn1nc2cccc(B3OC(C)(C)C(C)(C)O3)c2c1CCCN1C(=O)c2ccccc2C1=O]

The title compound was prepared according to the following scheme:

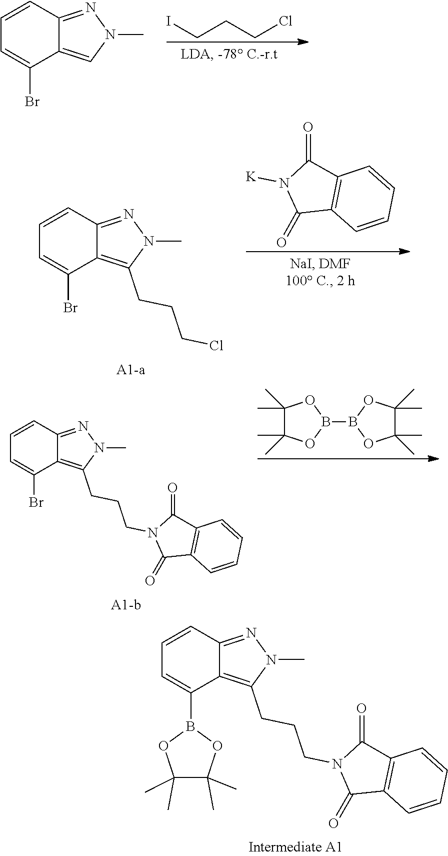

### Step 1: Preparation of 4-bromo-3-(3-chloropropyl)-2-methyl-indazole (Compound A1-a)

To a solution of 4-bromo-2-methyl-indazole (30.0 g, 142.1 mmol) in THF (400 mL) was added in one portion at −78° C. under nitrogen. And the LDA (107.1 mL, 214.3 mmol) was added dropwise into the mixture which was stirred for 1 hr. Then the 1-chloro-3-iodopropane (43.6 g, 213.2 mmol) was added dropwise at −78° C. After being stirred at −78° C. for 2 hrs, the reaction mixture was warmed to 25° C. for 12 hrs, then diluted with EtOAc, and poured into saturated ammonium chloride solution, extracted with EtOAc twice. The organic layer was washed with brine, dried over anhydrous Na₂SO₄. After filtration, the solvent was concentrated to afford crude product, which was purified by prep-HPLC to obtained compound A1-a (30 g) as a colorless solid, LCMS (M+H)⁺: 289.

### Step 2: Preparation of 2-[3-(4-bromo-2-methyl-indazol-3-yl)propyl]isoindoline-1,3-dione (Compound A1-b)

A mixture of 4-bromo-3-(3-chloropropyl)-2-methyl-indazole (Compound A1-a, 2 g, 3.8 mmol), potassium phthalimide (1.39 g, 7.5 mmol) and sodium iodide (1.69 g, 11.3 mmol) in DMF (30 mL) was heated at 100° C. for 2 hrs. After being cooled to room temperature, the reaction mixture was partitioned between water and EtOAc. The separated aqueous layer was extracted with EtOAc for three times, the combined organic layer was washed with brine, dried over anhydrous Na₂SO₄, concentrated under reduced pressure to give crude product. The crude precipitated from EtOAc/hexane to give crude compound A1-b (1.92 g) as a yellow solid, which was directly used in next step without further purification, LCMS (M+H)⁺: 398.

### Step 3: Preparation of 2-[3-[2-methyl-4-(4,4,5,5-tetramethyl-1,3,2-dioxaborolan-2-yl)indazol-3-yl]propyl]isoindoline-1,3-dione (Intermediate A1)

A mixture of 2-[3-(4-bromo-2-methyl-indazol-3-yl)propyl]isoindoline-1,3-dione (compound A1-b, 1.81 g, 3.4 mmol), bis(pinacolato)diboron (1.3 g, 5.1 mmol), bis(triphenylphosphine)palladium(ii) dichloride (125 mg, 0.17 mmol) and potassium acetate (1 g, 10.2 mmol) in 1,4-dioxane (30 mL) was heated at 100° C. for 16 hrs under argon. After being cooled to room temperature, the reaction mixture was diluted with water and EtOAc, then extracted with EtOAc for three times, the combined organic layer was washed with brine, dried over anhydrous Na₂SO₄, concentrated under reduced pressure to give crude product. The crude was purified by flash column to afford intermediate A1 (1.94 g) as a brown solid, LCMS (M+H)⁺: 446.

### Intermediate A2

### tert-Butyl N-methyl-N-[3-[2-methyl-4-(4,4,5,5-tetramethyl-1,3,2-dioxaborolan-2-yl)indazol-3-yl]propyl]carbamate

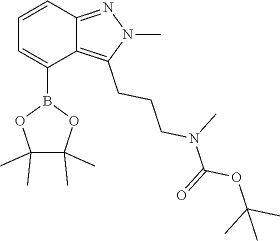 [SMILES: CN(CCCc1c2c(B3OC(C)(C)C(C)(C)O3)cccc2nn1C)C(=O)OC(C)(C)C]

The title compound was prepared according to the following scheme:

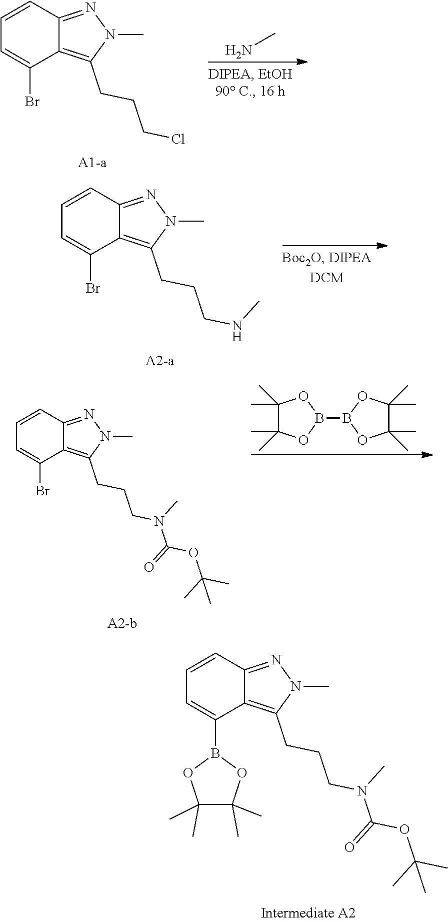

### Step 1: Preparation of 3-(4-bromo-2-methyl-indazol-3-yl)-N-methyl-propan-1-amine (Compound A2-a)

To a mixture of 4-bromo-3-(3-chloropropyl)-2-methyl-indazole (Compound A1-a, 6 g, 20.9 mmol) and DIPEA (5.39 g, 41.7 mmol) was added 33% methylamine in ethanol solution (40 mL), the resulting mixture was stirred at 90° C. for 16 hrs, then the reaction was concentrated to give crude compound A2-a (6.0 g) as an off-white solid, LCMS (M+H)⁺: 282.

### Step 2: Preparation of tert-butyl N-[3-(4-bromo-2-methyl-indazol-3-yl)propyl]-N-methyl-carbamate (Compound A2-b)

A mixture of 3-(4-bromo-2-methyl-indazol-3-yl)-N-methyl-propan-1-amine (compound A2-a, 6 g, 21.3 mmol), DIPEA (8.24 g, 63.8 mmol) and Boc anhydride (5.1 g, 23.4 mmol) in DCM (100 mL) was stirred at room temperature for 1 hr, then the reaction was concentrated and the residue was purified by flash column to give compound A2-b (8.0 g) as a colorless oil, LCMS (M+H)⁺: 382.

### Step 3: Preparation of tert-butyl N-methyl-N-[3-[2-methyl-4-(4,4,5,5-tetramethyl-1,3,2-dioxaborolan-2-yl)indazol-3-yl]propyl]carbamate (Intermediate A2)

A mixture of tert-butyl N-[3-(4-bromo-2-methyl-indazol-3-yl)propyl]-N-methyl-carbamate (compound A2-b, 6 g, 15.7 mmol), bis(pinacolato)diboron (7.97 g, 31.4 mmol) and potassium acetate (3.08 g, 31.4 mmol) in DMSO (73 mL) was bubbled with nitrogen for 5 mins and then bis(triphenylphosphine)palladium(ii) dichloride (1.1 g, 1.6 mmol) and butyldi-1-adamantylphosphine (1.13 g, 3.1 mmol) were added. The resulting mixture was heated at 130° C. 4 hrs. The reaction mixture was diluted with water, and then extracted with EtOAc twice. The combined organic layer was dried over anhydrous Na₂SO₄, concentrated to give a light brown oil. The crude was purified by flash column to give intermediate A2 (4.0 g) as a light brown oil, LCMS (M+H)⁺: 430.

### Intermediate A3

### tert-Butyl N-[3-[6-fluoro-2-methyl-4-(4,4,5,5-tetramethyl-1,3,2-dioxaborolan-2-yl)indazol-3-yl]propyl]-N-methyl-carbamate

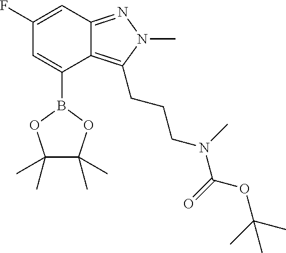 [SMILES: CN(CCCc1c2c(B3OC(C)(C)C(C)(C)O3)cc(F)cc2nn1C)C(=O)OC(C)(C)C]

The title compound was prepared according to the following scheme:

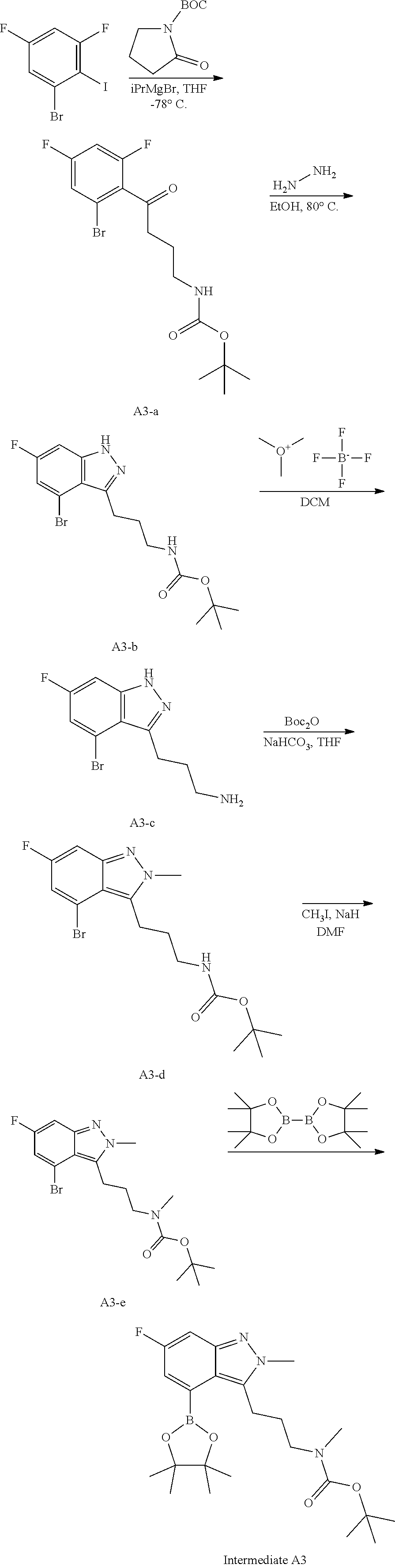

### Step 1: Preparation of tert-butyl N-[4-(2-bromo-4,6-difluoro-phenyl)-4-oxo-butyl]carbamate (Compound A3-a)

To a solution of 1-bromo-3,5-difluoro-2-iodo-benzene (100 g, 314 mmol) in THF (1000 mL) was added isopropyl magnesium bromide solution (157 mL, 314 mmol) dropwise at −45° C. After being stirred at −45° C. for 1 hr, the mixture was cooled to −78° C. and then tert-butyl 2-oxopyrrolidine-1-carboxylate (58.1 g, 314 mmol) was added at −78° C. Then the resulting mixture was warmed to room temperature slowly and stirred at room temperature for 4 hrs. The reaction mixture was quenched by saturated ammonium chloride solution at 0° C., diluted with water and extracted with EtOAc for three times. The combined organic layer was washed with brine, dried over anhydrous Na₂SO₄, filtered and concentrated under reduced pressure to give a red oil. The red oil was purified by flash column to give desired compound A3-a (76.0 g) as a red oil, LCMS (M+Na)⁺: 402.

### Step 2: Preparation of tert-butyl N-[3-(4-bromo-6-fluoro-1H-indazol-3-yl)propyl]carbamate (Compound A3-b)

To a solution of tert-butyl N-[4-(2-bromo-4,6-difluoro-phenyl)-4-oxo-butyl]carbamate (compound A3-a, 68.0 g, 180 mmol) in EtOH (700 mL) was added hydrazine hydrate (46.0 g, 899 mmol). The reaction mixture was stirred at 80° C. for 12 hrs. After being cooled to room temperature, the reaction mixture was diluted with water at 0° C., and then extracted with EtOAc for three times. The combined organic layer was washed with brine, dried over anhydrous Na₂SO₄, filtered and concentrated under reduced pressure to give a red oil, which was purified by flash column to give desired compound A3-b (25.0 g, 67.2 mmol, 37.4% yield) as a red oil, LCMS (M+H)⁺: 372.

### Step 3: Preparation of 3-(4-bromo-6-fluoro-2-methyl-indazol-3-yl)propan-1-amine (Compound A3-c)

To a solution of tert-butyl N-[3-(4-bromo-6-fluoro-1H-indazol-3-yl)propyl]carbamate (compound A3-b, 10.0 g, 26.9 mmol) in DCM (150 mL) was added trimethyloxonium tetrafluoroborate (5.17 g, 34.9 mmol), the resulting mixture was stirred at room temperature for 12 hrs. The reaction mixture was quenched by saturated sodium bicarbonate aqueous solution and then basified by saturated sodium carbonate aqueous solution to pH=9. The mixture was extracted with EtOAc for four times, the combined organic layer was washed with brine, dried over anhydrous Na₂SO₄, filtered and concentrated under reduced pressure to afford crude compound A3-c (8.00 g) as a red oil, which was directly used in next step without further purification, LCMS (M+H)⁺: 286.

### Step 4: Preparation of tert-butyl N-[3-(4-bromo-6-fluoro-2-methyl-indazol-3-yl)propyl]carbamate (Compound A3-d)

To a solution of 3-(4-bromo-6-fluoro-2-methyl-indazol-3-yl)propan-1-amine (compound A3-c, 10.0 g, 34.9 mmol) in THF (50 mL) was added Boc anhydride (7.63 g, 34.9 mmol) and NaHCO₃ (108 g, 1.29 mol), the reaction mixture was stirred at room temperature for 1 hr. The mixture was diluted with water and extracted with EtOAc for three times. The combined organic layer was washed with brine, dried over anhydrous Na₂SO₄, filtered and concentrated under reduced pressure to give some residue. The residue was purified by flash column to give crude compound A3-d (11.8 g) as a yellow solid, which was directly used in next step without further purification, LCMS (M+H)⁺: 386.

### Step 5: Preparation of tert-butyl N-[3-(4-bromo-6-fluoro-2-methyl-indazol-3-yl)propyl]-N-methyl-carbamate (Compound A3-e)

To a mixture of tert-butyl N-[3-(4-bromo-6-fluoro-2-methyl-indazol-3-yl)propyl]carbamate (Compound A3-d, 773 mg, 2 mmol) and iodomethane (852 mg, 6 mmol) in anhydrous DMF (10 mL) was added sodium hydride (320 mg, 8 mmol) in portions, the resulting mixture was stirred at room temperature for 16 hrs. The reaction was quenched by water, and then extracted with EtOAc for three times. The combined organic layers was washed with water, brine, dried over anhydrous Na₂SO₄, concentrated to give crude compound A3-e (1.02 g) as a yellow oil, which was directly used in next step without purification, LCMS (M+H)⁺: 400.

### Step 6: Preparation of tert-butyl N-[3-[6-fluoro-2-methyl-4-(4,4,5,5-tetramethyl-1,3,2-dioxaborolan-2-yl)indazol-3-yl]propyl]-N-methyl-carbamate (Intermediate A3)

A mixture of tert-butyl N-[3-(4-bromo-6-fluoro-2-methyl-indazol-3-yl)propyl]-N-methyl-carbamate (Compound A3-e, 856 mg, 1.7 mmol), bis(pinacolato)diborane (869 mg, 3.4 mmol), Pd(dppf)Cl₂ (125 mg, 0.17 mmol) and potassium acetate (504 mg, 5.1 mmol) in anhydrous 1,4-dioxane (10 mL) and anhydrous DMSO (1 mL) was heated at 110° C. for 16 hrs under nitrogen. After being cooled to room temperature, the reaction mixture was partitioned between water and EtOAc. The separated aqueous layer was extracted with EtOAc twice, the combined organic layer was washed with brine, dried over anhydrous Na₂SO₄, concentrated to give crude product. The crude was purified by flash column to afford intermediate A3 (841 mg) as a brown oil, which was directly used in next step without further purification, LCMS (M+H)⁺: 448.

### Intermediate A4 tert-Butyl N-[3-[7-fluoro-2-methyl-4-(4,4,5,5-tetramethyl-1,3,2-dioxaborolan-2-yl)indazol-3-yl]propyl]-N-methyl-carbamate

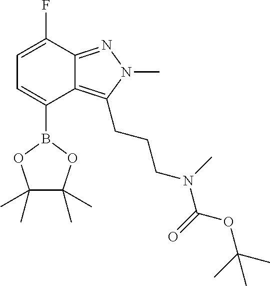 [SMILES: CN(CCCc1c2c(B3OC(C)(C)C(C)(C)O3)ccc(F)c2nn1C)C(=O)OC(C)(C)C]

The title compound was prepared according to the following scheme:

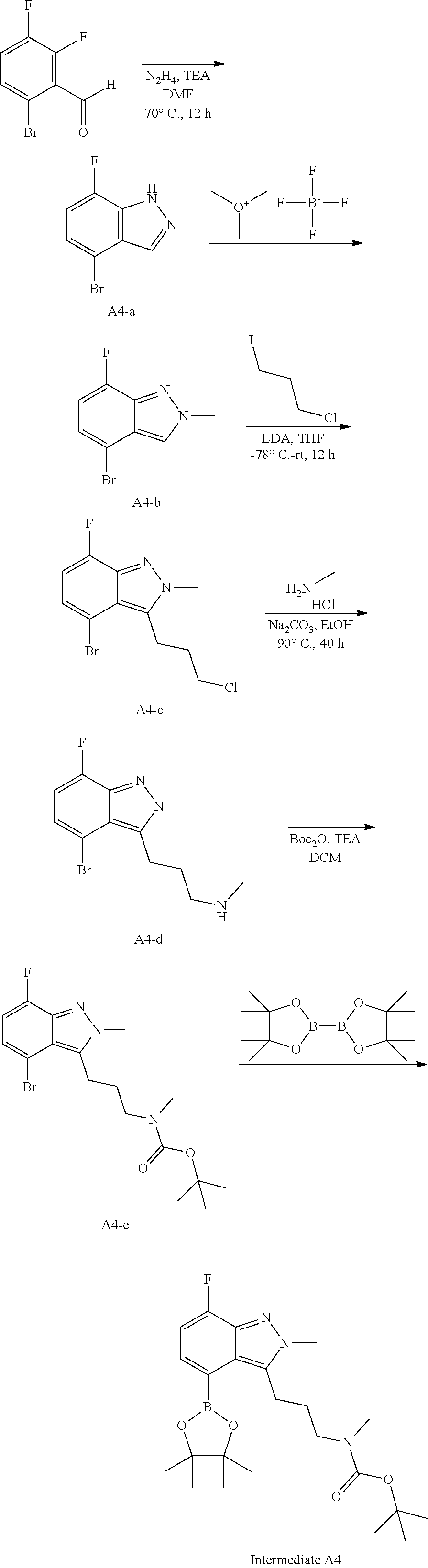

### Step 1: Preparation of 4-bromo-7-fluoro-1H-indazole (Compound A4-a)

To a mixture of 6-bromo-2,3-difluorobenzaldehyde (23.0 g, 104.1 mmol) and TEA (21.0 mL, 150.7 mmol) in anhydrous DMF (500 mL) was added hydrazine hydrate (50.0 g, 1361 mmol), the resulting mixture was stirred at 70° C. for 12 hrs. The mixture was diluted with EtOAc, and washed with water. The separated organic layer was washed with brine, then dried over anhydrous Na₂SO₄, filtered, concentrated to give crude product, which was purified by prep-HPLC to give compound A4-a (15.0 g) as a white solid, LCMS (M+H)⁺: 215.

### Step 2: Preparation of 4-bromo-7-fluoro-2-methyl-indazole (Compound A4-b)

To a solution of 4-bromo-7-fluoro-1H-indazole (Compound A4-a, 10.0 g, 46.5 mmol) in EtOAc (140 mL), trimethyloxonium tetrafluoroborate (11.6 g, 79.1 mmol) was added in one portion, the resulting mixture was stirred at room temperature for 12 hrs. The reaction mixture was diluted with EtOAc, and poured into water, then extracted with EtOAc twice. The combined organic layer was washed with brine, dried over anhydrous Na₂SO₄. After filtration, the filtrate was concentrated under reduced pressure to get crude product. The crude was purified by flash column to afford compound A4-b (8.0 g) as a yellow solid, LCMS (M+H)⁺: 229.

### Step 3: Preparation of 4-bromo-3-(3-chloropropyl)-7-fluoro-2-methyl-indazole (Compound A4-c)

To a solution of 4-bromo-7-fluoro-2-methyl-indazole (Compound A4-b, 8.0 g, 34.9 mmol) in THF (120 mL) was added LDA (26.2 mL, 52.4 mmol) dropwise for 10 mins at −78° C. under nitrogen, then 1-chloro-3-iodopropane (10.7 g, 52.4 mmol) was added dropwise at −78° C. After being stirred at −78° C. for 2 hrs, the mixture was warmed to room temperature for 12 hrs. The reaction mixture was diluted with EtOAc, poured into saturated ammonium chloride solution, and then extracted with EtOAc twice. The combined organic layer was washed with brine, dried over anhydrous Na₂SO₄. After filtration, the filtrate was concentrated under reduced pressure to get crude product, which was purified by prep-HPLC to afford compound A4-c (5.2 g) as a light brown solid, LCMS (M+H)⁺: 305.

### Step 4: Preparation of 3-(4-bromo-7-fluoro-2-methyl-indazol-3-yl)-N-methyl-propan-1-amine (Compound A4-d)

A mixture of 4-bromo-3-(3-chloropropyl)-7-fluoro-2-methyl-indazole (Compound A4-c, 611 mg, 2 mmol), methylamine hydrochloride (1.35 g, 20 mmol) and sodium carbonate (1.27 g, 12 mmol) in anhydrous EtOH (8 mL) was heated at 90° C. for 40 hrs. After being cooled to room temperature, the reaction mixture was partitioned between water and DCM. The separated aqueous layer was extracted with DCM twice, and then the combined organic layer was washed with brine, dried over anhydrous Na₂SO₄, concentrated to give crude compound A4-d (635 mg) as a yellow oil, which was directly used in next step without purification, LCMS (M+H)⁺: 300.

### Step 5: Preparation of tert-butyl N-[3-(4-bromo-7-fluoro-2-methyl-indazol-3-yl)propyl]-N-methyl-carbamate (Compound A4-e)

To a mixture of 3-(4-bromo-7-fluoro-2-methyl-indazol-3-yl)-N-methyl-propan-1-amine (compound A4-d, 635 mg, 1.7 mmol) and TEA (514 mg, 5.1 mmol) in DCM (10 mL) was added Boc anhydride (443 mg, 2.0 mmol), the resulting mixture was stirred at room temperature for 2 hrs. The reaction mixture was partitioned between water and EtOAc. The separated aqueous layer was extracted with EtOAc twice, the combined organic layer was washed with brine, dried over anhydrous Na₂SO₄, concentrated to give some residue. The residue was purified by flash column to afford compound A4-e (615 mg) as a colorless oil, LCMS (M+H)⁺: 400.

### Step 6: Preparation of tert-butyl N-[3-[7-fluoro-2-methyl-4-(4,4,5,5-tetramethyl-1,3,2-dioxaborolan-2-yl)indazol-3-yl]propyl]-N-methyl-carbamate (Intermediate A4)

A mixture of tert-butyl N-[3-(4-bromo-7-fluoro-2-methyl-indazol-3-yl)propyl]-N-methyl-carbamate (Compound A4-e, 596 mg, 1.5 mmol), bis(pinacolato)diboron (945 mg, 3.7 mmol), Pd(dppf)Cl₂ (97 mg, 0.15 mmol) and potassium acetate (292 mg, 3.0 mmol) in anhydrous 1,4-dioxane (10 mL) was heated at 100° C. for 16 hrs under nitrogen. After being cooled to room temperature, the reaction mixture was partitioned between water and EtOAc, the separated aqueous layer was extracted with EtOAc twice. The combined organic layer was washed with brine, dried over anhydrous Na₂SO₄, concentrated in vacuo to give crude product, the crude was purified by flash column to afford intermediate A4 (320 mg) as a light brown oil, LCMS (M+H)⁺: 448.

### Intermediate A5 tert-Butyl N-(3-hydroxypropyl)-N-[3-[2-methyl-4-(4,4,5,5-tetramethyl-1,3,2-dioxaborolan-2-yl)indazol-3-yl]propyl]carbamate

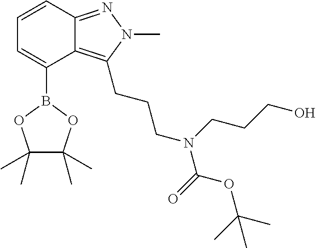 [SMILES: Cn1nc2cccc(B3OC(C)(C)C(C)(C)O3)c2c1CCCN(CCCO)C(=O)OC(C)(C)C]

The title compound was prepared according to the following scheme:

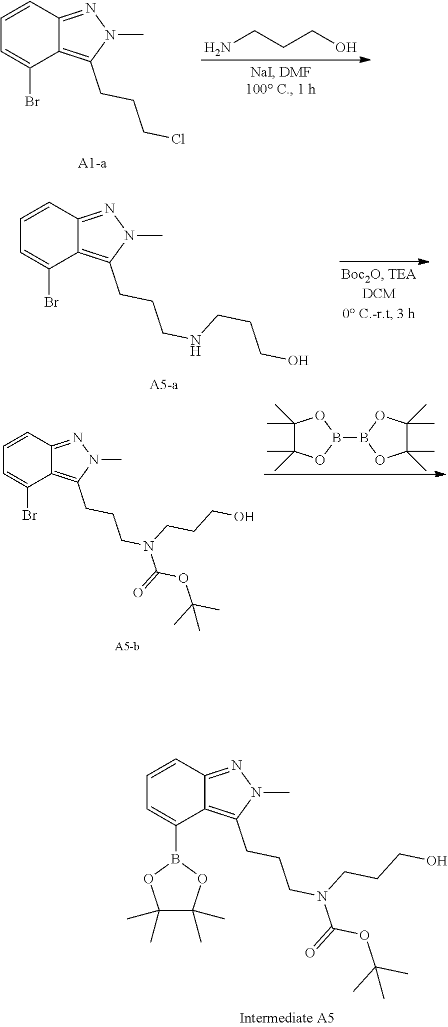

### Step 1: Preparation of 3-[3-(4-bromo-2-methyl-indazol-3-yl)propylamino]propan-1-ol (Compound A5-a)

A mixture of 4-bromo-3-(3-chloropropyl)-2-methyl-indazole (Compound A1-a, 1.44 g, 5 mmol), 3-aminopropan-1-ol (3.76 g, 50 mmol) and sodium iodide (1.5 g, 10 mmol) in DMF (15 mL) was heated to 100° C. for 1 hr. The reaction mixture was partitioned between water and DCM, then the separated aqueous layer was extracted with DCM for three times. The combined organic layer was washed with brine, dried over anhydrous Na₂SO₄, concentrated under reduced pressure to give crude compound A5-a (3.49 g) as a yellow oil, which was directly used in next step without further purification, LCMS (M+H)⁺: 326.

### Step 2: Preparation of tert-butyl N-[3-(4-bromo-2-methyl-indazol-3-yl)propyl]-N-(3-hydroxypropyl)carbamate (Compound A5-b)

To a mixture of 3-[3-(4-bromo-2-methyl-indazol-3-yl)propylamino]propan-1-ol (compound A5-a, 3.49 g, 4.8 mmol) and TEA (584 mg, 5.8 mmol) in DCM (30 mL) was added Boc anhydride (3.15 g, 14.4 mmol) at 0° C., the resulting mixture was stirred at 0° C. to room temperature for 3 hrs. The reaction mixture was partitioned between water and DCM, the separated aqueous layer was extracted with DCM twice. The combined organic layer was washed with brine, dried over anhydrous Na₂SO₄, concentrated in vacuo to give crude product. The crude was purified by flash column to afford compound A5-b (1.42 g) as a yellow solid, which was directly used in next step without further purification, LCMS (M+H)⁺: 428.

### Step 3: Preparation of tert-butyl N-(3-hydroxypropyl)-N-[3-[2-methyl-4-(4,4,5,5-tetramethyl-1,3,2-dioxaborolan-2-yl)indazol-3-yl]propyl]carbamate (Intermediate A5)

A mixture of tert-butyl N-[3-(4-bromo-2-methyl-indazol-3-yl)propyl]-N-(3-hydroxypropyl)carbamate (A5-b, 1.42 g, 3.3 mmol), bis(pinacolato)diboron (1.27 g, 5.0 mmol), Pd(dppf)Cl₂ (136 mg, 0.17 mmol) and potassium acetate (654 mg, 6.7 mmol) in anhydrous 1,4-dioxane (20 mL) was heated at 110° C. for 16 hrs under nitrogen. After being cooled to room temperature, the reaction mixture was partitioned between water and DCM, the separated aqueous layer was extracted with DCM twice. The combined organic layer was washed with brine, dried over anhydrous Na₂SO₄, concentrated in vacuo to give crude product, the crude was purified by flash column to afford desired intermediate A5 (1.23 g) as a brown oil, LCMS (M+H)⁺: 474.

### Intermediate A6

### N-[(4-methoxyphenyl)methyl]-N-methyl-3-[2-methyl-4-(4,4,5,5-tetramethyl-1,3,2-dioxaborolan-2-yl)indazol-3-yl]propan-1-amine

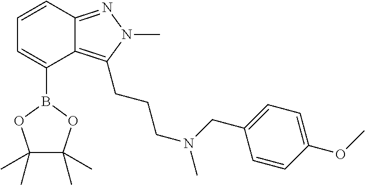

The title compound was prepared according to the following scheme:

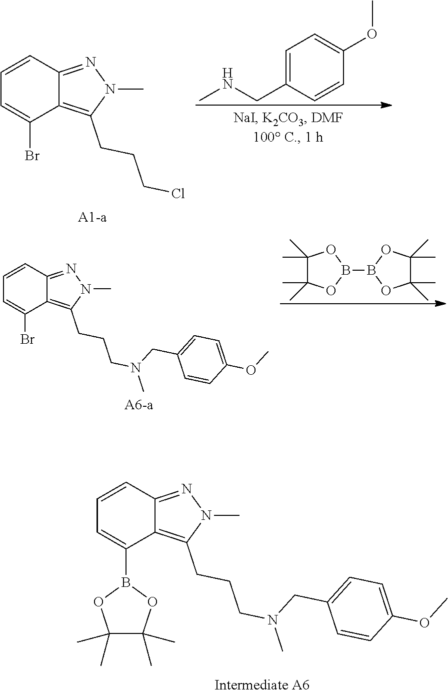

### Step 1: Preparation of 3-(4-bromo-2-methyl-indazol-3-yl)-N-[(4-methoxyphenyl)methyl]-N-methyl-propan-1-amine (Compound A6-a)

A mixture of 4-bromo-3-(3-chloropropyl)-2-methyl-indazole (Compound A1-a, 3.0 g, 10.4 mmol), 4-methoxy-N-methylbenzylamine (2.0 g, 13.2 mmol), sodium iodide (2.0 g, 13.3 mmol) and potassium carbonate (2.0 g, 14.47 mmol) in anhydrous DMF (30 mL) was stirred at 120° C. for 2 hrs. The mixture was cooled to room temperature, and then poured into water. The resulted precipitate was filtered, the filter cake was dissolved in EtOAc. Then the resulting solution was dried over anhydrous Na₂SO₄, concentrated to give crude compound A6-a (3.7 g) as a yellow oil, which was used in next step directly, LCMS (M+H)⁺: 402.

### Step 2: Preparation of N-[(4-methoxyphenyl)methyl]-N-methyl-3-[2-methyl-4-(4,4,5,5-tetramethyl-1,3,2-dioxaborolan-2-yl)indazol-3-yl]propan-1-amine (Intermediate A6)

To a solution of 3-(4-bromo-2-methyl-indazol-3-yl)-N-[(4-methoxyphenyl)methyl]-N-methyl-propan-1-amine (Compound A6-a, 3.7 g, 9.2 mmol), bis(pinacolato)diboron (4.5 g, 17.7 mmol) and potassium phosphate (4.0 g, 18.8 mmol) in 1,4-dioxane (30 mL) was added cataCXium A-Pd-G2 (0.5 g, 0.75 mmol), the resulting mixture was stirred at 100° C. for 12 hrs under argon. The mixture was diluted with EtOAc, and poured into water, then extracted with EtOAc. The separated organic layer was washed brine and dried over anhydrous Na₂SO₄, filtered, concentrated to give crude product, which was purified by prep-HPLC to give intermediate A6 (2.3 g) as a yellow oil, which was used in next step directly, LCMS (M+H)⁺: 450.

### Intermediate A7

### tert-Butyl N-[3-[7-fluoro-2-methyl-4-(4,4,5,5-tetramethyl-1,3,2-dioxaborolan-2-yl)indazol-3-yl]propyl]-N-(3-morpholinopropyl)carbamate

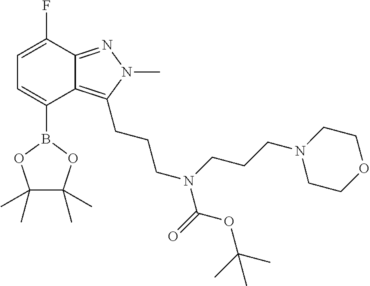 [SMILES: Cn1nc2c(F)ccc(B3OC(C)(C)C(C)(C)O3)c2c1CCCN(CCCN1CCOCC1)C(=O)OC(C)(C)C]

The title compound was prepared according to the following scheme:

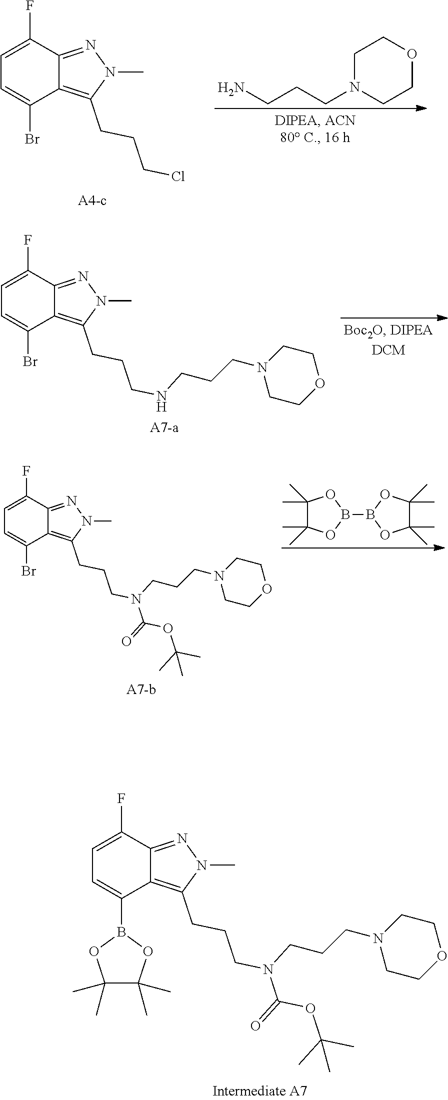

### Step 1: Preparation of 3-(4-bromo-7-fluoro-2-methyl-indazol-3-yl)-N-(3-morpholinopropyl)propan-1-amine (Compound A7-a)

A mixture of 4-bromo-3-(3-chloropropyl)-7-fluoro-2-methyl-indazole (Compound A4-c, 917 mg, 3 mmol), N-(3-aminopropyl)morpholine (2.16 g, 15 mmol) and DIPEA (1.16 g, 9 mmol) in anhydrous ACN (2 mL) was heated at 80° C. for 16 hrs. After being cooled to room temperature, the reaction mixture was partitioned between water and EtOAc, the separated aqueous layer was extracted with EtOAc twice. The combined organic layer was washed with brine, dried over anhydrous Na₂SO₄, concentrated in vacuo to give compound A7-a (1.39 g) as a light brown oil, which was directly used in next step without purification, LCMS (M+H)⁺: 413.

### Step 2: Preparation of tert-butyl N-[3-(4-bromo-7-fluoro-2-methyl-indazol-3-yl)propyl]-N-(3-morpholinopropyl)carbamate (Compound A7-b)

A mixture of 3-(4-bromo-7-fluoro-2-methyl-indazol-3-yl)-N-(3-morpholinopropyl)propan-1-amine (Compound A7-a, 1.39 g, 2.7 mmol), Boc anhydride (881 mg, 4.0 mmol) and DIPEA (1.04 g, 8.1 mmol) in DCM (10 mL) was stirred at room temperature for 16 hrs. The reaction mixture was concentrated in vacuo to give crude product, the crude was purified by flash column to afford compound A7-b (1.48 g) as a light yellow oil, LCMS (M+H)⁺: 513.

### Step 3: Preparation of tert-butyl N-[3-[7-fluoro-2-methyl-4-(4,4,5,5-tetramethyl-1,3,2-dioxaborolan-2-yl)indazol-3-yl]propyl]-N-(3-morpholinopropyl)carbamate (Intermediate A7)

A mixture of tert-butyl N-[3-(4-bromo-7-fluoro-2-methyl-indazol-3-yl)propyl]-N-(3-morpholinopropyl)carbamate (Compound A7-b, 1.48 g, 2.6 mmol), bis(pinacolato)diboron (1.65 g, 6.5 mmol), Pd(dppf)Cl₂·DCM adduct (214 mg, 0.26 mmol) and potassium acetate (764 mg, 7.8 mmol) in anhydrous 1,4-dioxane (15 mL) was heated at 110° C. for 16 hrs under nitrogen. After being cooled to room temperature, the reaction mixture was partitioned between water and EtOAc, the separated aqueous layer was extracted with EtOAc twice. The combined organic layer was washed with water, brine, dried over anhydrous Na₂SO₄, concentrated in vacuo to give crude product, the crude was purified by flash column to afford intermediate A7 (890 mg) as a brown oil, which was directly used in next step without further purification, LCMS (M+H)⁺: 561.

### Intermediate B1

### O1-tert-butyl O2-methyl (2S,4S)-4-(3-bromophenoxy)pyrrolidine-1,2-dicarboxylate

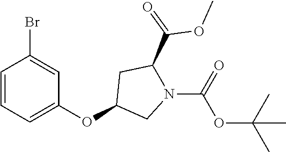 [SMILES: COC(=O)[C@@H]1C[C@H](Oc2cccc(Br)c2)CN1C(=O)OC(C)(C)C]

The title compound was prepared according to the following scheme:

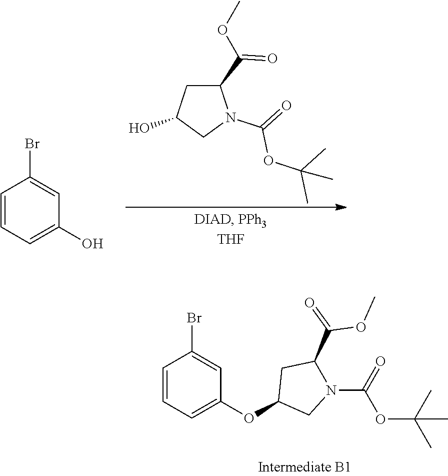 [SMILES: Oc1cccc(Br)c1 ; COC(=O)[C@@H]1C[C@@H](O)CN1C(=O)OC(C)(C)C ; COC(=O)[C@@H]1C[C@H](Oc2cccc(Br)c2)CN1C(=O)OC(C)(C)C]

To a mixture of 3-bromophenol (4.23 g, 24.5 mmol), O1-tert-butyl O2-methyl (2S,4R)-4-hydroxypyrrolidine-1,2-dicarboxylate (4 g, 16.3 mmol), PPh₃ (6.42 g, 24.5 mmol) in THF (5 mL) was added DIAD (4.95 g, 24.5 mmol) dropwise. The resulting mixture was stirred at room temperature for 4 hrs. The mixture was concentrated and the residue was purified by flash column to afford intermediate B1 (5.5 g), LCMS (M+H)⁺: 400.

### Intermediate B2

### O1-tert-butyl O2-methyl (2S,4S)-4-[3-(4,4,5,5-tetramethyl-1,3,2-dioxaborolan-2-yl)phenoxy]pyrrolidine-1,2-dicarboxylate

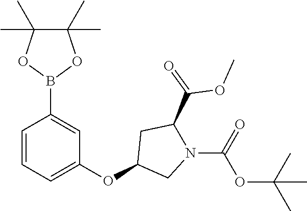 [SMILES: COC(=O)[C@@H]1C[C@H](Oc2cccc(B3OC(C)(C)C(C)(C)O3)c2)CN1C(=O)OC(C)(C)C]

To a stirred solution of 1-(tert-butyl) 2-methyl (2S,4R)-4-hydroxypyrrolidine-1,2-dicarboxylate (2.0 g, 8.2 mmol), 3-(4,4,5,5-tetramethyl-1,3,2-dioxaborolan-2-yl)phenol (1.79 g, 8.2 mmol) and PPh₃ (2.46 g, 9.4 mmol) in THF (50 mL) was added DIAD (1.9 g, 9.4 mmol) in THF (10 mL) dropwise at 0° C. After being stirred at room temperature overnight, the reaction mixture was concentrated to give some residue, the residue was purified by silica gel to give intermediate B2, LCMS: (M+H)⁺: 448.

### Intermediate B3

### O1-tert-butyl O2-methyl (2S,4S)-4-[(6-bromo-2-pyridyl)amino]pyrrolidine-1,2-dicarboxylate

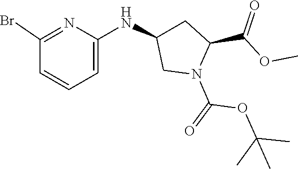 [SMILES: COC(=O)[C@@H]1C[C@H](Nc2cccc(Br)n2)CN1C(=O)OC(C)(C)C]

A mixture of 1-(tert-butyl) 2-methyl (2S,4S)-4-aminopyrrolidine-1,2-dicarboxylate (28.0 g, 99.7 mmol), DIPEA (69.5 mL, 398.9 mmol) and 2-bromo-6-fluoropyridine (21.1 g, 119.7 mmol) in DMSO (140 mL) was heated at 110° C. for 16 hrs. The mixture was diluted with water, and extracted with EtOAc, the separated organic layer was concentrated to give intermediate B3 (34 g), LCMS (M+H⁺): 400.

### Intermediate B4

### O1-benzyl O2-methyl (2S,4S)-4-[(6-bromo-2-pyridyl)amino]pyrrolidine-1,2-dicarboxylate

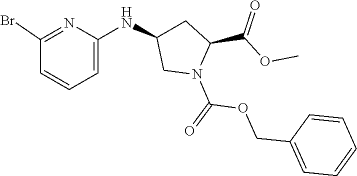 [SMILES: COC(=O)[C@@H]1C[C@H](Nc2cccc(Br)n2)CN1C(=O)OCc1ccccc1]

A mixture of O1-benzyl O2-methyl (2S,4S)-4-aminopyrrolidine-1,2-dicarboxylate;hydrochloride (4.7 g, 14.9 mmol), 2-bromo-6-fluoropyridine (3.15 g, 17.9 mmol) and DIPEA (13.0 mL, 74.7 mmol) in DMSO (20 mL) was stirred at 110° C. for 16 hrs. The mixture was concentrated to give some residue, the residue was purified by prep-HPLC to give intermediate B4 (5.1 g), LCMS (M+H⁺): 434.

### Intermediate B5

### O1-benzyl O2-methyl (2S,4S)-4-[(6-bromo-4-methyl-2-pyridyl)amino]pyrrolidine-1,2-dicarboxylate

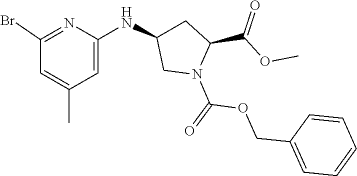 [SMILES: COC(=O)[C@@H]1C[C@H](Nc2cc(C)cc(Br)n2)CN1C(=O)OCc1ccccc1]

A mixture of O1-benzyl O2-methyl (2S,4S)-4-aminopyrrolidine-1,2-dicarboxylate;hydrochloride (3.0 g, 9.5 mmol), 2-bromo-6-fluoro-4-methyl-pyridine (1.99 g, 10.5 mmol) and DIPEA (3.7 g, 28.6 mmol) in DMSO (30 mL) was stirred at 110° C. for 16 hrs. The reaction mixture was cooled to room temperature, then diluted with EtOAc, washed with water, the separated organic layer was dried over anhydrous Na₂SO₄ and concentrated under reduced pressure to give some residue, the residue was purified by flash column to give intermediate B5 (2.2 g), LCMS (M+H)⁺: 448.

### Intermediate B6

### O1-tert-butyl O2-methyl (2S,4S)-4-[(6-bromo-4-methoxy-2-pyridyl)amino]pyrrolidine-1,2-dicarboxylate

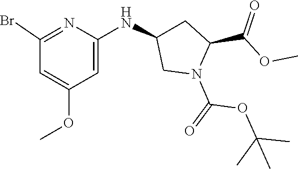 [SMILES: COC(=O)[C@@H]1C[C@H](Nc2cc(OC)cc(Br)n2)CN1C(=O)OC(C)(C)C]

A mixture of O1-benzyl O2-methyl (2S,4S)-4-aminopyrrolidine-1,2-dicarboxylate;hydrochloride (4.0 g, 14.2 mmol), 2,6-dibromo-4-methoxypyridine (4.4 g, 16.5 mmol), Pd₂(dba)₃ (2.17 g, 2.4 mmol), XantPhos (1.37 g, 2.4 mmol) and cesium carbonate (13.9 g, 42.7 mmol) in 1,4-dioxane (80 mL) was stirred at 85° C. for 12 hrs under nitrogen. The reaction mixture was poured into water, and extracted with EtOAc for three times, the combined organic layer was washed with brine, dried over Na₂SO₄, filtered and evaporated to give a crude residue. The residue was purified by flash column to give intermediate B6 (2.0 g) as a yellow oil, LCMS (M+H)⁺: 430.

### Intermediate B7 and B8

### O1-tert-butyl O2-methyl (2S,4S)-4-[(4-chloropyrimidin-2-yl)amino]pyrrolidine-1,2-dicarboxylate and O1-tert-butyl O2-methyl (2S,4S)-4-[(2-chloropyrimidin-4-yl)amino]pyrrolidine-1,2-dicarboxylate

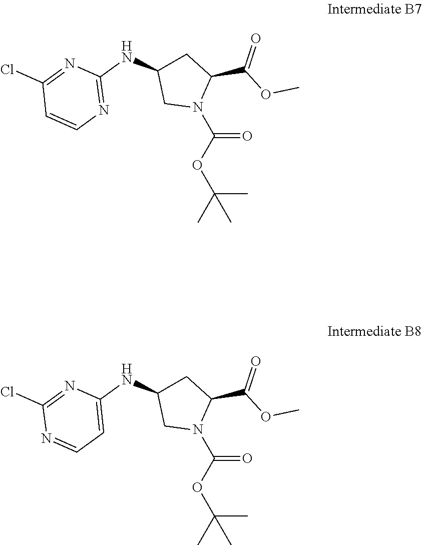 [SMILES: COC(=O)[C@@H]1C[C@H](Nc2nccc(Cl)n2)CN1C(=O)OC(C)(C)C ; COC(=O)[C@@H]1C[C@H](Nc2ccnc(Cl)n2)CN1C(=O)OC(C)(C)C]

The title compounds were prepared according to the following scheme:

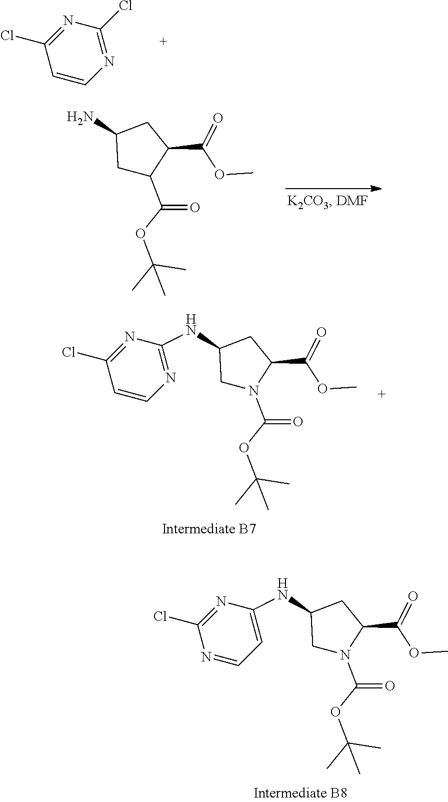

A mixture of 2,4-dichloropyrimidine (19.2 g, 128.9 mmol), 1-(tert-butyl) 2-methyl (2S,4S)-4-aminopyrrolidine-1,2-dicarboxylate (21 g, 86.0 mmol) and potassium carbonate (35.64 g, 257.9 mmol) in DMF (100 mL) was stirred at 80° C. for 16 hrs. Then the reaction was diluted with EtOAc, washed with water and brine, the separated organic layer was concentrated to give some residue, the residue was purified by flash column to give intermediate B7 (faster eluted, 5 g), LCMS (M+H)⁺: 357; and intermediate B8 (slower eluted, 25 g), LCMS (M+H)⁺: 357.

### Intermediate B9

### O1-tert-butyl O2-methyl (2S,4S)-4-[(6-bromopyrazin-2-yl)amino]pyrrolidine-1,2-dicarboxylate

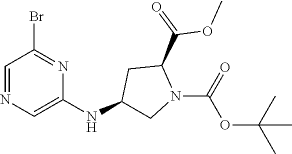 [SMILES: COC(=O)[C@@H]1C[C@H](Nc2cncc(Br)n2)CN1C(=O)OC(C)(C)C]

A mixture of 2-bromo-6-fluoro-pyrazine (3.12 g, 17.6 mmol), O1-tert-butyl O2-methyl (2S,4S)-4-aminopyrrolidine-1,2-dicarboxylate hydrochloride (4.5 g, 16.0 mmol) and DIPEA (6.21 g, 48.1 mmol) in DMSO was heated at 110° C. for 12 hrs. The mixture was diluted with EtOAc, washed with water, the separated organic layer was dried over anhydrous Na₂SO₄ and concentrated to give intermediate B9 (7 g), LCMS (M+H)⁺: 401.

### Intermediate B10

### O1-tert-butyl O2-methyl (2S,4S)-4-[(6-bromo-2-pyridyl)oxy]pyrrolidine-1,2-dicarboxylate

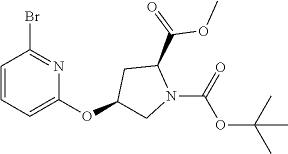 [SMILES: COC(=O)[C@@H]1C[C@H](Oc2cccc(Br)n2)CN1C(=O)OC(C)(C)C]

To a mixture of 2-bromo-6-hydroxypyridine (2.0 g, 11.5 mmol), O1-tert-butyl O2-methyl (2S,4R)-4-hydroxypyrrolidine-1,2-dicarboxylate (3.7 g, 14.9 mmol) and PPh₃ (6.0 g, 23.0 mmol) in anhydrous THF (10 mL) was added DIAD (3.5 g, 17.2 mmol) at 0° C. under nitrogen. The resulting mixture was stirred at 70° C. for 1 hr with microwave irritation. The mixture was concentrated under reduced pressure to give crude product, which was purified by chromatography on silica gel to give intermediate B10 (1.84 g) as a white solid, LCMS (M+Na)⁺: 423.

### Intermediate B11

### O1-tert-butyl O2-methyl (2S,4S)-4-[(2-bromo-4-pyridyl)oxy]pyrrolidine-1,2-dicarboxylate

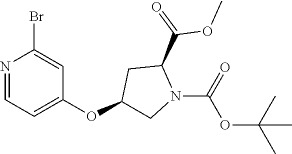 [SMILES: COC(=O)[C@@H]1C[C@H](Oc2ccnc(Br)c2)CN1C(=O)OC(C)(C)C]

To a mixture of O1-tert-butyl O2-methyl (2S,4R)-4-hydroxypyrrolidine-1,2-dicarboxylate (5.2 g, 21.2 mmol), 2-bromo-4-hydroxypyridine (5.53 g, 31.8 mmol) and PPh₃ (8.3 g, 31.8 mmol) in anhydrous toluene (50 mL) was added DIAD (6.43 g, 31.8 mmol) at 0° C. under nitrogen. The resulting mixture was stirred at 80° C. for 16 hrs. The reaction mixture was cooled to room temperature and then concentrated under reduced pressure to give crude product, which was purified by prep-HPLC to give intermediate B11 (2.80 g) as a light brown oil, LCMS (M+H)⁺: 401.

### Intermediate B12

### O1-tert-butyl O2-methyl (2S,4S)-4-(4-bromopyrimidin-2-yl)oxypyrrolidine-1,2-dicarboxylate

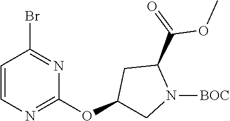 [SMILES: COC(=O)[C@@H]1C[C@H](Oc2nccc(Br)n2)CN1C(=O)OC(C)(C)C]

The title compound was prepared according to the following scheme:

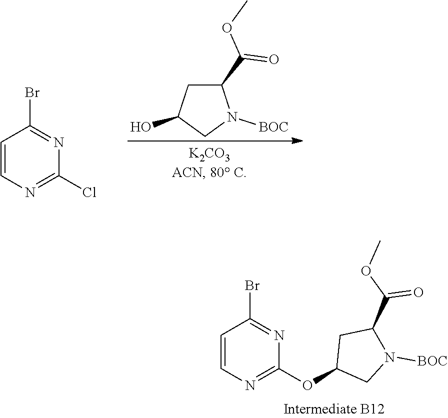 [SMILES: Clc1nccc(Br)n1 ; COC(=O)[C@@H]1C[C@H](O)CN1C(=O)OC(C)(C)C ; COC(=O)[C@@H]1C[C@H](Oc2nccc(Br)n2)CN1C(=O)OC(C)(C)C]

To a solution of O1-tert-butyl O2-methyl (2S,4S)-4-hydroxypyrrolidine-1,2-dicarboxylate (5.0 g, 20.4 mmol), potassium carbonate (8.45 g, 61.2 mmol) in ACN (100 mL) was added 4-bromo-2-chloropyrimidine (3.94 g, 20.4 mmol) and then stirred at 80° C. for 16 hrs. The reaction mixture was poured into saturated ammonium chloride solution, and then extracted with EtOAc for three times. The combined organic layer was washed with brine, dried over anhydrous Na₂SO₄, filtered and evaporated to afford crude product, which was purified by flash column to afford intermediate B12 (2.69 g), LCMS (M-100+H)⁺: 302.

### Intermediate B13

### O1-benzyl O2-methyl (2S,4S)-4-(4-chloropyrimidin-2-yl)oxypyrrolidine-1,2-dicarboxylate

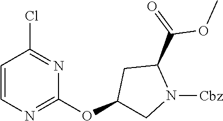 [SMILES: COC(=O)[C@@H]1C[C@H](Oc2nccc(Cl)n2)CN1C(=O)OCc1ccccc1]

To a mixture of O1-benzyl O2-methyl (2S,4S)-4-hydroxypyrrolidine-1,2-dicarboxylate (30.0 g, 107.4 mmol) and 4-chloro-2-methylsulfonyl-pyrimidine (20.0 g, 103.8 mmol) in anhydrous THF (340 mL) was added sodium hydride (60% in oil, 4.4 g, 110 mmol) in portions at room temperature under nitrogen, the resulting mixture was stirred at room temperature for 16 hrs. The mixture was poured into saturated ammonium chloride solution, and extracted with EtOAc. The separated organic layer was dried over anhydrous Na₂SO₄ and concentrated to give crude product, which was purified by flash column to give intermediate B13 (27.0 g), LCMS (M+H)⁺: 392.

### Intermediate B14

### O1-tert-butyl O2-methyl (2S,4S)-4-(6-chloropyrimidin-4-yl)oxypyrrolidine-1,2-dicarboxylate

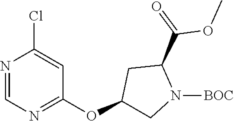 [SMILES: COC(=O)[C@@H]1C[C@H](Oc2cc(Cl)ncn2)CN1C(=O)OC(C)(C)C]

To a solution of O1-tert-butyl O2-methyl (2S,4S)-4-hydroxypyrrolidine-1,2-dicarboxylate (10.0 g, 40.8 mmol) in anhydrous DMF (200 mL) was added sodium hydride (4.89 g, 122.3 mmol) at 0° C., 30 mins later, 4,6-dichloropyrimidine (6.07 g, 40.8 mmol) was added at 0° C. and then stirred at room temperature for 2 hrs. The reaction mixture was quenched with saturated ammonium chloride solution, and extracted with EtOAc for three times. The combined organic layer was washed with brine and dried over anhydrous Na₂SO₄, concentrated under reduced pressure to give crude product, which was purified by flash column to afford intermediate B14 (11.0 g) as a colorless oil, LCMS (M-100+H)⁺: 302.

### Intermediate B15

### O1-tert-butyl O2-methyl (2S,4S)-4-[(6-chloro-2-pyridyl)amino]pyrrolidine-1,2-dicarboxylate

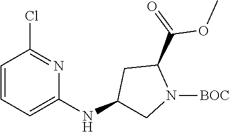 [SMILES: COC(=O)[C@@H]1C[C@H](Nc2cccc(Cl)n2)CN1C(=O)OC(C)(C)C]

To a solution of O1-tert-butyl O2-methyl (2S,4S)-4-aminopyrrolidine-1,2-dicarboxylate hydrochloride (5.0 g, 17.8 mmol) and 2,6-dichloropyridine (3.0 g, 20.3 mmol) in 1,4-dioxane (50 mL) was added XPhos (3.4 g, 7.1 mmol), Pd₂(dba)₃ (3.26 g, 3.6 mmol) and Cesium carbonate (11.6 g, 35.6 mmol). The resulting mixture was stirred at 90° C. for 16 hrs, and then the reaction mixture was filtered, the filtrate was concentrated under reduced pressure to give some residue, the residue was purified by flash column to give intermediate B15 (2.5 g) as a yellow solid, LCMS (M+H)⁺: 300.

### Intermediate C1

### 4-Chloro-1-(2,4-difluorophenyl)pyrazolo[3,4-d]pyrimidine

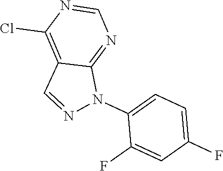 [SMILES: Fc1ccc(-n2ncc3c(Cl)ncnc32)c(F)c1]

The title compound was prepared according to the following scheme:

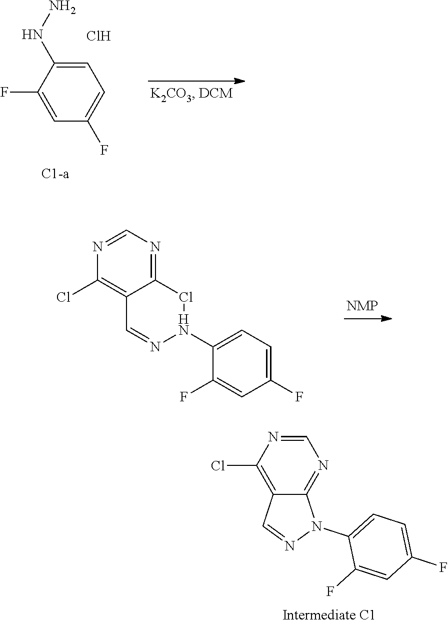

### Step 1: Preparation of N-[(4,6-dichloropyrimidin-5-yl)methyleneamino]-2,4-difluoro-aniline (Compound C1-b)

A mixture of 4,6-dichloro-5-pyrimidinecarbaldehyde (20.0 g, 113 mmol), 2,4-difluorophenylhydrazine hydrochloride (compound C1-a, 24.9 g, 138.0 mmol) and potassium carbonate (21.2 g, 153.2 mmol) in DCM (400 mL) was stirred at room temperature for 12 hrs. Then the mixture was washed with water and brine, the separated organic layer was dried over anhydrous Na₂SO₄ and concentrated to give the crude compound C1-b (34.5 g), LCMS (M+H)⁺: 303.

### Step 2: Preparation of 4-chloro-1-(2,4-difluorophenyl)pyrazolo[3,4-d]pyrimidine (Intermediate C1)

To a solution of N-[(4,6-dichloropyrimidin-5-yl)methyleneamino]-2,4-difluoro-aniline (compound C1-b, 18.5 g, 61.0 mmol) in NMP (185 mL) was added 4 A molecular sieve (2 g). The reaction mixture was stirred at 110° C. for 40 hrs. The reaction was cooled and 300 mL of water was added into the reaction mixture within 20 mins. The resulted precipitate was collected and dried to give intermediate C1 (8.5 g), LCMS (M+H)⁺: 267.

### Intermediate C2

### 4-Chloro-1-(4-fluoro-2-methoxy-phenyl)pyrazolo[3,4-d]pyrimidine

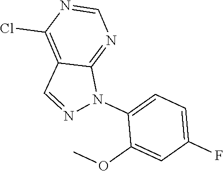 [SMILES: COc1cc(F)ccc1-n1ncc2c(Cl)ncnc21]

The title compound was prepared according to the following scheme:

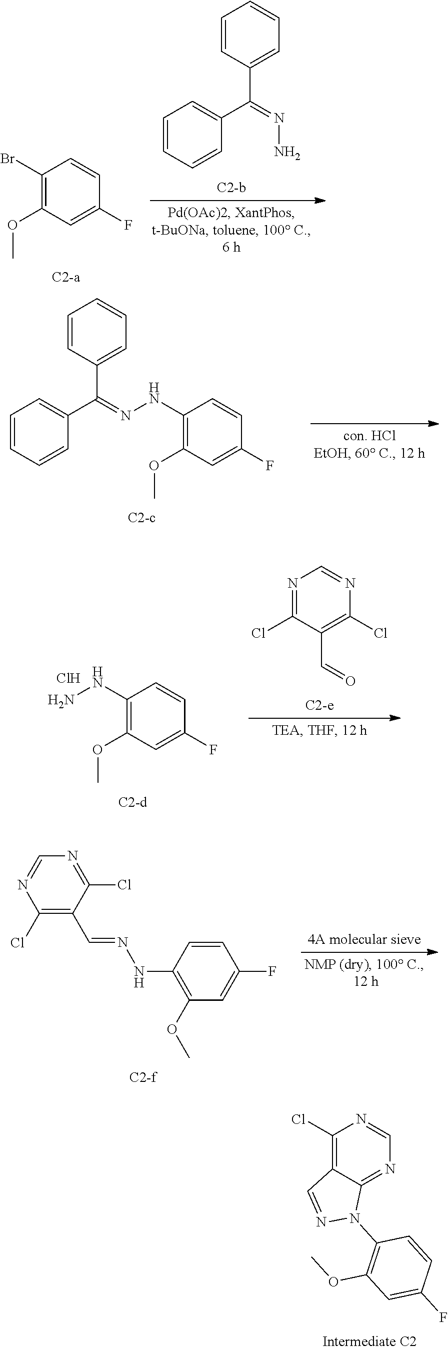

### Step 1: Preparation of N-(benzhydrylideneamino)-4-fluoro-2-methoxy-aniline (Compound C2-c)

A solution of 2-bromo-5-fluoroanisole (compound C2-a, 10.0 g, 48.8 mmol), benzophenone hydrazone (compound C2-b, 10.0 g, 51.0 mmol), palladium (II) acetate (200 mg, 0.90 mmol), XantPhos (400 mg, 0.70 mmol) and sodium tert-butoxide (6.7 g, 69.7 mmol) in anhydrous toluene (150 mL) was stirred at 100° C. for 12 hrs under nitrogen. The mixture was diluted with EtOAc, washed with water and brine. The organic layer was dried and concentrated to give crude product, which was recrystallized from petroleum ether to give compound C2-c (12.0 g), which was used in next step directly, LCMS (M+H)⁺: 321.

### Step 2: Preparation of (4-fluoro-2-methoxy-phenyl)hydrazine hydrochloride (Compound C2-d)

A mixture of compound N-(benzhydrylideneamino)-4-fluoro-2-methoxy-aniline (compound C2-c, 11.0 g, 34.3 mmol) in EtOH (100 mL) and con. HCl (10 mL, 120 mmol) was stirred at 60° C. for 12 hrs. The mixture was concentrated, and then EtOAc was added. After stirring for 5 mins, the mixture was filtered, the filter cake was washed with petroleum ether and dried to give compound C2-d (4.5 g), which was used in next step directly, LCMS (M+H)⁺: 141.

### Step 3: Preparation of N-[(4,6-dichloropyrimidin-5-yl)methyleneamino]-4-fluoro-2-methoxy-aniline (Compound C2-f)

A mixture of (4-fluoro-2-methoxy-phenyl)hydrazine hydrochloride (compound C2-d, 4.3 g, 22.3 mmol), 4,6-dichloro-5-pyrimidinecarbaldehyde (C2-e, 4.3 g, 24.3 mmol) and TEA (3.5 g, 34.6 mmol) in anhydrous THF (100 mL) was stirred at room temperature for 12 hrs. The reaction mixture was diluted with EtOAc, washed with water and brine. The separated organic layer was dried over anhydrous Na₂SO₄ and concentrated to give crude compound C2-f (5.2 g), which was used in next step directly, LCMS (M+H)⁺: 315.

### Step 4: Preparation of 4-chloro-1-(4-fluoro-2-methoxy-phenyl)pyrazolo[3,4-d]pyrimidine (Intermediate C2)

A mixture of N-[(4,6-dichloropyrimidin-5-yl)methyleneamino]-4-fluoro-2-methoxy-aniline (compound C2-f, 4.0 g, 12.7 mmol) and 4 A molecular sieve (1.0 g) in anhydrous DMF (10 mL) was stirred at 100° C. for 12 hrs. The mixture was diluted with EtOAc, washed with water and brine. The separated organic layer was dried over anhydrous Na₂SO₄ and concentrated to give crude product, which was purified by prep-TLC to give intermediate C2 (747 mg), LCMS (M+H)⁺: 279.

### Intermediate C3

### 8-Chloro-3-(2,4-difluorophenyl)imidazo[1,5-a]pyrazine

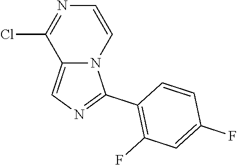 [SMILES: Fc1ccc(-c2ncc3c(Cl)nccn23)c(F)c1]

The title compound was prepared according to the following scheme:

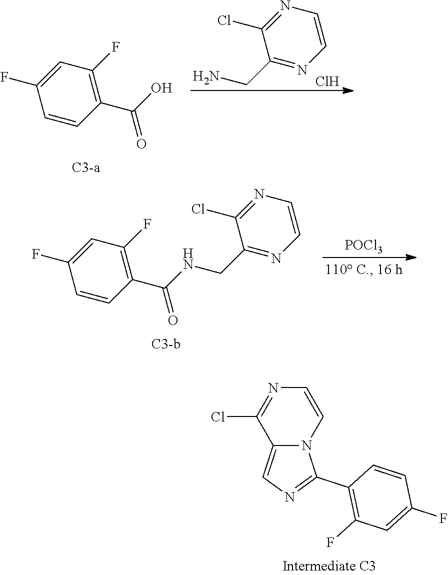

### Step 1: Preparation of N-[(3-chloropyrazin-2-yl)methyl]-2,4-difluoro-benzamide (Compound C3-b)

To a mixture of 2,4-difluorobenzoic acid (C3-a, 8.0 g, 50.6 mmol) in DCM (100 mL) was added thionyl chloride (260 mL) at room temperature. After being stirred at 80° C. for 2 hrs, the mixture was concentrated and diluted with DCM. Then to the solution was added (3-chloropyrazin-2-yl)methanamine hydrochloride (9.12 g, 50.7 mmol) and TEA (14.3 mL, 102.8 mmol) at 0° C. The mixture was stirred at room temperature for 1 hr. Then the mixture was concentrated to give some residue, the residue was diluted with DCM, washed with water and brine. The separated organic layer was dried over anhydrous Na₂SO₄ and concentrated to afford compound C3-b (7.62 g), LCMS (M+H)⁺: 284.

### Step 2: Preparation of 8-chloro-3-(2,4-difluorophenyl)imidazo[1,5-a]pyrazine (Intermediate C3)

To a solution of N-[(3-chloropyrazin-2-yl)methyl]-2,4-difluoro-benzamide (compound C3-b, 16.2 g, 57.1 mmol) in toluene (320 mL) was added phosphorus oxychloride (26.6 mL, 285.6 mmol). The mixture was stirred at 110° C. for 16 hrs. Then the mixture was concentrated to give some residue, the residue was added to a vigorous stirring mixture of EtOAc (300 mL, contained 10 mL of TEA) and 50 g sodium bicarbonate in 300 mL water at 0° C. The organic layer was separated, dried over anhydrous Na₂SO₄ and concentrated to give intermediate C3 (15.4 g), LCMS (M+H)⁺: 266.

### Example 1

### (8S,11S)-10-[1-(2,4-difluorophenyl)pyrazolo[3,4-d]pyrimidin-4-yl]-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one

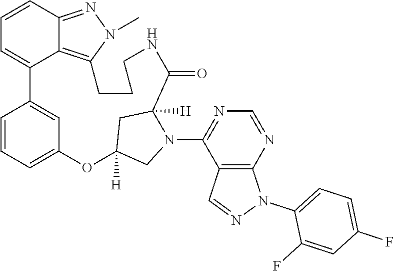 [SMILES: Cn1nc2cccc3c2c1CCCNC(=O)[C@@H]1C[C@@H](CN1c1ncnc2c1cnn2-c1ccc(F)cc1F)Oc1cccc-3c1]

The title compound was prepared according to the following scheme:

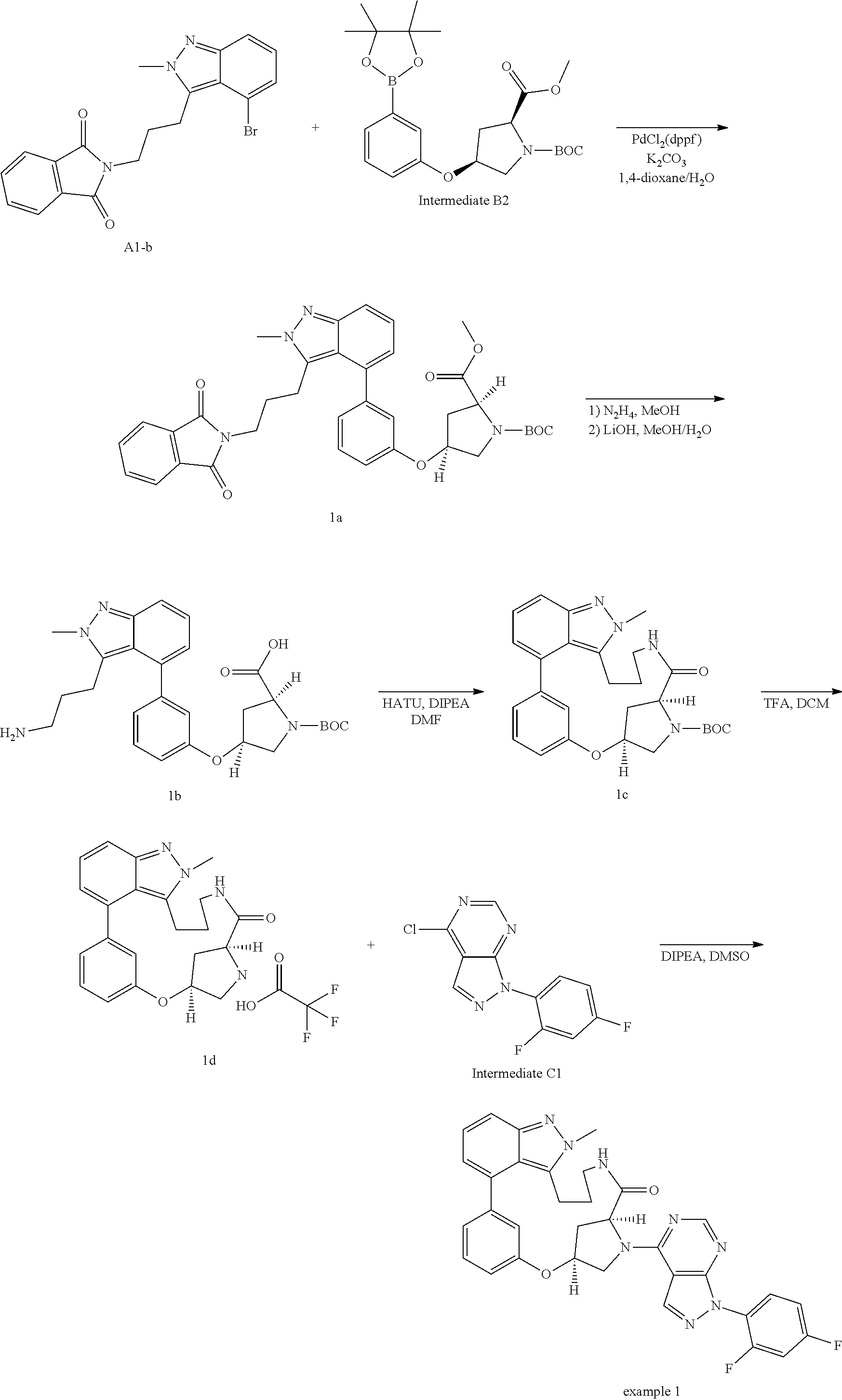

### Step 1: Preparation of O1-tert-butyl O2-methyl (2S,4S)-4-[3-[3-[3-(1,3-dioxoisoindolin-2-yl)propyl]-2-methyl-indazol-4-yl]phenoxy]pyrrolidine-1,2-dicarboxylate (Compound 1a)

A mixture of 2-[3-(4-bromo-2-methyl-indazol-3-yl)propyl]isoindoline-1,3-dione (compound A1-b, 2.5 g, 6.3 mmol), O1-tert-butyl O2-methyl (2S,4S)-4-[3-(4,4,5,5-tetramethyl-1,3,2-dioxaborolan-2-yl)phenoxy]pyrrolidine-1,2-dicarboxylate (intermediate B2, 3.65 g, 8.2 mmol), Pd(dppf)Cl₂·DCM (230 mg, 0.3 mol) and potassium carbonate (2.6 g, 18.8 mmol) in 1,4-dioxane (50 mL) and water (5 mL) was heated at 100° C. for 16 hrs under argon. After being cooled to room temperature, the reaction mixture was partitioned between water and EtOAc. The separated aqueous layer was extracted with EtOAc for three times, the combined organic layer was washed with water, brine, dried over anhydrous Na₂SO₄, concentrated under reduced pressure to give crude product. The crude was purified by flash column to afford compound 1a (3.35 g) as a light yellow solid, LCMS (M+H)⁺: 639.

### Step 2: Preparation of (2S,4S)-4-[3-[3-(3-aminopropyl)-2-methyl-indazol-4-yl]phenoxy]-1-tert-butoxycarbonyl-pyrrolidine-2-carboxylic acid (Compound 1b)

A mixture of O1-tert-butyl O2-methyl (2S,4S)-4-[3-[3-[3-(1,3-dioxoisoindolin-2-yl)propyl]-2-methyl-indazol-4-yl]phenoxy]pyrrolidine-1,2-dicarboxylate (compound 1a, 3.3 g, 4.1 mmol) and hydrazine hydrate (1.03 g, 20.7 mmol) in MeOH (60 mL) was heated at 70° C. for 16 hrs in a sealed tube. After being cooled to room temperature, the reaction mixture was diluted with water, then extracted with EtOAc for three times. The combine organic layer was washed with brine, dried over anhydrous Na₂SO₄, concentrated in vacuo to give some yellow oil. The yellow oil was dissolved in MeOH (28 mL) and water (7 mL), then lithium hydroxide monohydrate (759 mg, 18.1 mmol) was added. The resulting mixture was stirred at room temperature for 12 hrs, and then diluted with water, acidified by HOAc, extracted with DCM for three times. The combined organic layer was washed with water, brine, dried over anhydrous Na₂SO₄, concentrated under reduced pressure to afford crude compound 1b (2.35 g) as a light yellow solid, LCMS (M+H)⁺: 495.

### Step 3: Preparation of tert-butyl (8S,11S)-18-methyl-12-oxo-7-oxa-10,13,18,19-tetrazapentacyclo[15.6.1.1^{2,6}.1^{8,11}.0^{20,24}]hexacosa-1(23),2(26),3,5,17(24),19,21-heptaene-10-carboxylate (Compound 1c)

To a mixture of DIPEA (4.0 g, 31.0 mmol) and HATU (8.0 g, 42.0 mmol) in DMF (1.5 L) was added a solution of (2S,4S)-4-[3-[3-(3-aminopropyl)-2-methyl-indazol-4-yl]phenoxy]-1-tert-butoxycarbonyl-pyrrolidine-2-carboxylic acid (compound 1b, 8.0 g, 16.2 mmol) in DMF (1.5 L) at 0° C. dropwise and then stirred at 0° C. for 2 hrs. The mixture was concentrated to give some residue, which was diluted with EtOAc, then washed with water, 0.5 N aq. HCl solution, saturated sodium bicarbonate aqueous solution and brine successively. The separated organic layer was dried over anhydrous Na₂SO₄, filtered, the filtrate was concentrated under reduced pressure to give crude compound 1c (5.2 g) as a yellow solid, which was used in next step directly, LCMS (M+H)⁺: 477.

### Step 4: Preparation of (8S,11S)-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;2,2,2-trifluoroacetic acid (Compound 1d)

A mixture of tert-butyl (8S,11S)-18-methyl-12-oxo-7-oxa-10,13,18,19-tetrazapentacyclo[15.6.1.1^{2,6}.1^{8,11}.0^{20,24}]hexacosa-1(23),2(26),3,5,17(24),19,21-heptaene-10-carboxylate (compound 1c, 2.38 g, 5 mmol) and TFA (5 mL) in DCM (20 mL) was stirred at room temperature for 3 hrs. The reaction mixture was concentrated under reduced pressure to afford crude compound 1d (3.27 g) as a brown oil, LCMS (M+H)⁺: 377.

### Step 5: Preparation of (8S,11S)-10-[1-(2,4-difluorophenyl)pyrazolo[3,4-d]pyrimidin-4-yl]-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one (Example 1)

A mixture of (8S,11S)-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;2,2,2-trifluoroacetic acid (compound 1d, 3.27 g, 5 mmol), 4-chloro-1-(2,4-difluorophenyl)pyrazolo[3,4-d]pyrimidine (intermediate C1, 2 g, 6 mmol) and DIPEA (3.23 g, 25 mmol) in DMSO (30 mL) was heated at 90° C. for 1 hr. After being cooled to room temperature, the reaction mixture was diluted with water and some precipitation formed. The precipitate was filtered, the filter cake was collected and dried to give crude product. The crude product was slurried in MeOH, then filtered, the filter cake was collected, Example 1 (2.65 g) was obtained as a yellow solid, LCMS (M+H)⁺: 607. ¹H NMR (400 MHz, METHANOL-d₄) δ=8.58-7.89 (m, 2H), 7.69-7.59 (m, 1H), 7.54-7.43 (m, 2H), 7.33-7.25 (m, 2H), 7.24-7.17 (m, 1H), 7.16-7.08 (m, 2H), 7.02 (d, J=7.8 Hz, 1H), 6.94 (dd, J=0.6, 6.8 Hz, 1H), 5.55-5.35 (m, 1H), 5.16-4.95 (m, 1H), 4.63-4.19 (m, 2H), 4.11 (s, 3H), 3.74-3.60 (m, 1H), 3.15-2.91 (m, 2H), 2.89-2.71 (m, 2H), 2.68-2.47 (m, 1H), 1.96-1.81 (m, 2H).

### Example 2

### (8S,11S)-10-[1-(2,4-difluorophenyl)pyrazolo[3,4-d]pyrimidin-4-yl]-13,18-dimethyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one

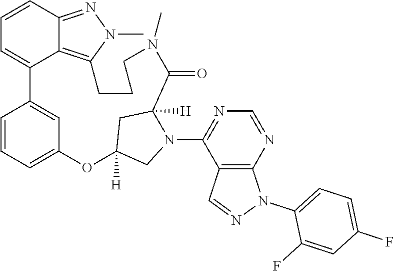 [SMILES: CN1CCCc2c3c(cccc3nn2C)-c2cccc(c2)O[C@H]2C[C@@H](C1=O)N(c1ncnc3c1cnn3-c1ccc(F)cc1F)C2]

The title compound was prepared according to the following scheme:

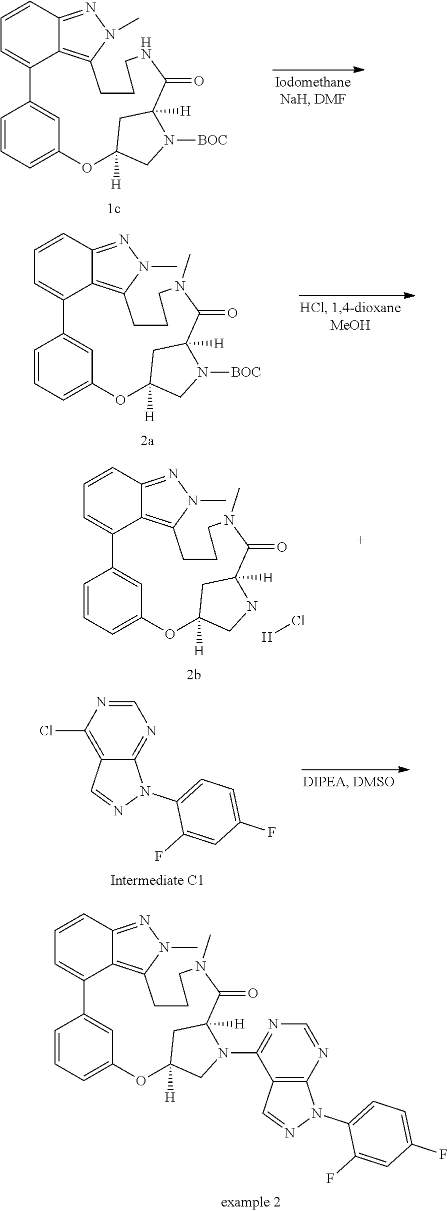

### Step 1: Preparation of tert-butyl (8S,11S)-13,18-dimethyl-12-oxo-7-oxa-10,13,18,19-tetrazapentacyclo[15.6.1.1^{2,6}.1^{8,11}.0^{20,24}]hexacosa-1(23),2(26),3,5,17(24),19,21-heptaene-10-carboxylate (Compound 2a)

To a solution of tert-butyl (8S,11S)-18-methyl-12-oxo-7-oxa-10,13,18,19-tetrazapentacyclo[15.6.1.1^{2,6}.1^{8,11}.0^{20,24}]hexacosa-1(23),2(26),3,5,17(24),19,21-heptaene-10-carboxylate (compound 1c, 118 mg, 0.17 mol) in DMF (3 mL) was added sodium hydride (35 mg, 0.87 mmol) at 0° C., 30 mins later, iodomethane (123 mg, 0.87 mmol) was added, the resulting mixture was stirred at 0° C. to room temperature for 16 hrs. The reaction mixture was partitioned between water and EtOAc. The separated aqueous layer was extracted with EtOAc for three times, the combined organic layer was washed with water, brine, dried over anhydrous Na₂SO₄, concentrated under reduced pressure to afford compound 2a (160 mg) as a yellow oil, LCMS (M+H)⁺: 491.

### Step 2: Preparation of (8S,11S)-13,18-dimethyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;hydrochloride (Compound 2b)

To a solution of tert-butyl (8S,11S)-13,18-dimethyl-12-oxo-7-oxa-10,13,18,19-tetrazapentacyclo[15.6.1.1^{2,6}.1^{8,11}.0^{20,24}]hexacosa-1(23),2(26),3,5,17(24),19,21-heptaene-10-carboxylate (compound 2a, 160 mg, 0.16 mmol) in MeOH (2 mL) was added 4 M HCl in 1,4-dioxane solution (3 mL), the resulting mixture was stirred at room temperature for 1 hr. After being concentrated under reduced pressure, crude compound 2b (108 mg) was obtained as a yellow solid, LCMS (M+H)⁺: 391.

### Step 3: Preparation of (8S,11S)-10-[1-(2,4-difluorophenyl)pyrazolo[3,4-d]pyrimidin-4-yl]-13,18-dimethyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one (Example 2)

A mixture of (8S,11S)-13,18-dimethyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;hydrochloride (compound 2b, 54 mg, 0.76 mmol), 4-chloro-1-(2,4-difluorophenyl)pyrazolo[3,4-d]pyrimidine (intermediate C1, 26 mg, 0.99 mmol) and DIPEA (49 mg, 0.38 mmol) in DMSO (1 mL) was heated at 100° C. for 2 hrs. After being cooled to room temperature, the reaction mixture was directly purified by prep-HPLC to afford example 2 (29 mg) as a yellow solid, LCMS (M+H)⁺: 621. ¹H NMR (400 MHz, METHANOL-d₄) δ=8.64-7.87 (m, 2H), 7.73-7.61 (m, 1H), 7.57-7.47 (m, 2H), 7.37-7.26 (m, 2H), 7.25-7.17 (m, 2H), 7.04-6.93 (m, 2H), 6.91-6.84 (m, 1H), 5.70-5.48 (m, 2H), 4.68-4.26 (m, 2H), 4.23-4.15 (m, 1H), 4.12 (s, 3H), 3.23-3.07 (m, 3H), 3.01-2.66 (m, 5H), 2.40-2.24 (m, 1H), 1.67-1.52 (m, 1H).

### Example 3

### (8S,11S)-10-[1-(2,4-difluorophenyl)pyrazolo[3,4-d]pyrimidin-4-yl]-13,18-dimethyl-7,10,13,18,19,26-hexazapentacyclo[15.6.1.1^{2,6}.1^{8,11}.0^{20,24}]hexacosa-1(23),2(26),3,5,17(24),19,21-heptaen-12-one

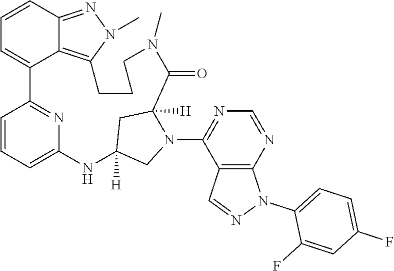 [SMILES: CN1CCCc2c3c(cccc3nn2C)-c2cccc(n2)N[C@H]2C[C@@H](C1=O)N(c1ncnc3c1cnn3-c1ccc(F)cc1F)C2]

The title compound was prepared according to the following scheme:

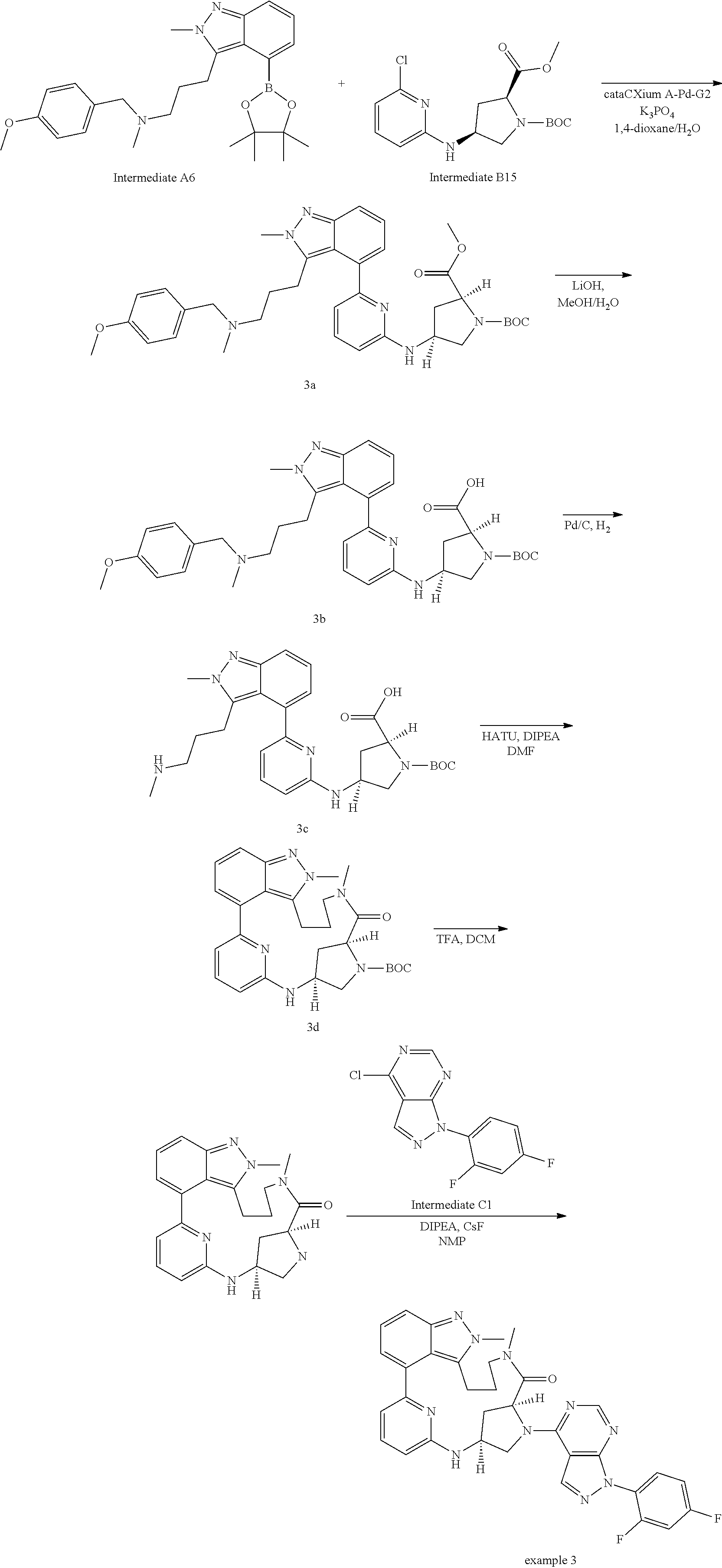

### Step 1: Preparation of O1-tert-butyl O2-methyl (2S,4S)-4-[[6-[3-[3-[(4-methoxyphenyl)methyl-methyl-amino]propyl]-2-methyl-indazol-4-yl]-2-pyridyl]amino]pyrrolidine-1,2-dicarboxylate (Compound 3a)

To a solution of N-[(4-methoxyphenyl)methyl]-N-methyl-3-[2-methyl-4-(4,4,5,5-tetramethyl-1,3,2-dioxaborolan-2-yl)indazol-3-yl]propan-1-amine (Intermediate A6, 1.39 g, 3.09 mmol) and O1-tert-butyl O2-methyl (2S,4S)-4-[(6-chloro-2-pyridyl)amino]pyrrolidine-1,2-dicarboxylate (intermediate B15, 1 g, 2.8 mmol) in 1,4-dioxane (10 mL) and water (1 mL) was added cataCXium A-Pd-G2 (282 mg, 0.42 mmol) and potassium phosphate (1.19 g, 5.62 mmol) under argon. The mixture was stirred at 80° C. for 2 hrs, then the reaction mixture was concentrated to give some residue, which was purified by flash column to give compound 3a (1.2 g) as a yellow solid, LCMS (M+H)⁺: 643.

### Step 2: Preparation of (2S,4S)-1-tert-butoxycarbonyl-4-[[6-[3-[3-[(4-methoxyphenyl)methyl-methyl-amino]propyl]-2-methyl-indazol-4-yl]-2-pyridyl]amino]pyrrolidine-2-carboxylic acid (Compound 3b)

To a solution of O1-tert-butyl O2-methyl (2S,4S)-4-[[6-[3-[3-[(4-methoxyphenyl)methyl-methyl-amino]propyl]-2-methyl-indazol-4-yl]-2-pyridyl]amino]pyrrolidine-1,2-dicarboxylate (compound 3a, 1.1 g, 1.71 mmol) in MeOH (22 mL) and water (11 mL) was added lithium hydroxide monohydrate (300 mg, 7.15 mmol). After being stirred at room temperature for 12 hrs, the reaction mixture was adjust to pH=4˜6 by 1 N aq. HCl solution. Then the mixture was extracted with EtOAc, the separated organic layer was washed with brine, dried over anhydrous Na₂SO₄ and concentrated under reduced pressure to give compound 3b (0.8 g) as a yellow solid, LCMS (M+H)⁺: 629.

### Step 3: Preparation of (2S,4S)-1-tert-butoxycarbonyl-4-[[6-[2-methyl-3-[3-(methylamino)propyl]indazol-4-yl]-2-pyridyl]amino]pyrrolidine-2-carboxylic acid (Compound 3c)

To a solution of (2S,4S)-1-tert-butoxycarbonyl-4-[[6-[3-[3-[(4-methoxyphenyl)methyl-methyl-amino]propyl]-2-methyl-indazol-4-yl]-2-pyridyl]amino]pyrrolidine-2-carboxylic acid (compound 3b, 200 mg, 0.32 mmol) in THF (5 mL) and isopropanol (5 mL) was added Pd/C (338 mg, 0.32 mmol) under nitrogen. The reaction mixture was stirred at 40° C. for 6 hrs under hydrogen, then filtered and concentrated under reduced pressure to give compound 3c (160 mg) as a yellow solid, LCMS (M+H)⁺: 509.

### Step 4: Preparation of tert-butyl (8S,11S)-13,18-dimethyl-12-oxo-7,10,13,18,19,26-hexazapentacyclo[15.6.1.1^{2,6}.1^{8,11}.0^{20,24}]hexacosa-1(23),2(26),3,5,17(24),19,21-heptaene-10-carboxylate (Compound 3d)

To a solution of HATU (448 mg, 1.18 mmol) and DIPEA (254 mg, 1.97 mmol) in DMF (100 mL) was added (2S,4S)-1-tert-butoxycarbonyl-4-[[6-[2-methyl-3-[3-(methylamino)propyl]indazol-4-yl]-2-pyridyl]amino]pyrrolidine-2-carboxylic acid (compound 3c, 500 mg, 0.98 mmol) in THF (100 mL) dropwise at −10° C. Then the reaction mixture was stirred at −10° C. for 1 hr, the mixture was concentrated under reduced pressure to give some residue, which was purified by flash column to give compound 3d (300 mg) as a yellow solid, LCMS (M+H)⁺: 491.

### Step 5: Preparation of (8S,11S)-13,18-dimethyl-7,10,13,18,19,26-hexazapentacyclo[15.6.1.1^{2,6}.1^{8,11}.0^{20,24}]hexacosa-1(23),2(26),3,5,17(24),19,21-heptaen-12-one (Compound 3e)

To a solution of tert-butyl (8S,11S)-13,18-dimethyl-12-oxo-7,10,13,18,19,26-hexazapentacyclo[15.6.1.1^{2,6}.1^{8,11}.0^{20,24}]hexacosa-1(23),2(26),3,5,17(24),19,21-heptaene-10-carboxylate (compound 3d, 30 mg, 0.06 mmol) in DCM (0.5 mL) was added TFA (70 mg, 0.61 mmol). The reaction mixture was stirred at room temperature for 2 hrs, then concentrated under reduced pressure to give compound 3e (20 mg) as a yellow solid, LCMS (M+H)⁺: 391.

### Step 6: Preparation of (8S,11S)-10-[1-(2,4-difluorophenyl)pyrazolo[3,4-d]pyrimidin-4-yl]-13,18-dimethyl-7,10,13,18,19,26-hexazapentacyclo[15.6.1.1^{2,6}.1^{8,11}.0^{20,24}]hexacosa-1(23),2(26),3,5,17(24),19,21-heptaen-12-one (Example 3)

To a solution of (8S,11S)-13,18-dimethyl-7,10,13,18,19,26-hexazapentacyclo[15.6.1.1^{2,6}.1^{8,11}.0^{20,24}]hexacosa-1(23),2(26),3,5,17(24),19,21-heptaen-12-one (compound 3e, 20 mg, 0.05 mmol) and 4-chloro-1-(2,4-difluorophenyl)pyrazolo[3,4-d]pyrimidine (Intermediate C1, 16 mg, 0.06 mmol) in NMP (0.2 mL) was added potassium fluoride (15 mg, 0.26 mmol) and DIPEA (66 mg, 0.51 mmol) under nitrogen. The mixture was stirred at 120° C. for 12 hrs, then cooled to room temperature, the reaction mixture was diluted with EtOAc, washed with water and brine. The separated organic layer was dried over anhydrous Na₂SO₄ and concentrated under reduced pressure to give some residue. The residue was purified by flash column to give example 3 (12 mg) as a white solid, LCMS (M+H)⁺: 621. ¹H NMR (400 MHz, METHANOL-d₄) δ=8.55 (s, 1H), 8.26 (s, 1H), 8.23-8.16 (m, 1H), 7.73 (d, J=8.7 Hz, 1H), 7.70-7.61 (m, 1H), 7.39 (dd, J=6.9, 8.7 Hz, 1H), 7.33-7.26 (m, 2H), 7.25-7.16 (m, 2H), 7.11-7.05 (m, 1H), 5.66-5.55 (m, 1H), 4.97-4.91 (m, 1H), 4.81-4.64 (m, 1H), 4.35 (d, J=11.9 Hz, 1H), 4.15 (s, 3H), 4.13-4.05 (m, 1H), 3.29-3.16 (m, 3H), 3.14-3.04 (m, 1H), 3.03-2.89 (m, 1H), 2.74 (ddd, J=5.0, 8.9, 13.8 Hz, 1H), 2.66-2.54 (m, 2H), 2.38-2.17 (m, 1H), 1.52-1.38 (m, 1H).

### Example 4

### (8S,11S)-10-[1-(2,4-difluorophenyl)pyrazolo[3,4-d]pyrimidin-4-yl]-13,18-dimethyl-5,7,10,13,18,19,26-heptazapentacyclo[15.6.1.1^{2,6}.1^{8,11}.0^{20,24}]hexacosa-1(23),2,4,6(26),17(24),19,21-heptaen-12-one

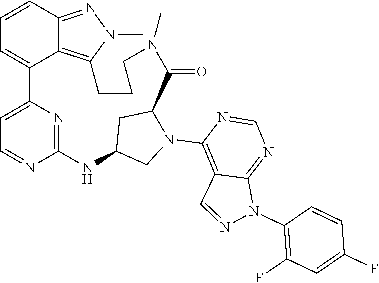 [SMILES: CN1CCCc2c3c(cccc3nn2C)-c2ccnc(n2)N[C@H]2C[C@@H](C1=O)N(c1ncnc3c1cnn3-c1ccc(F)cc1F)C2]

The title compound was prepared according to the following scheme:

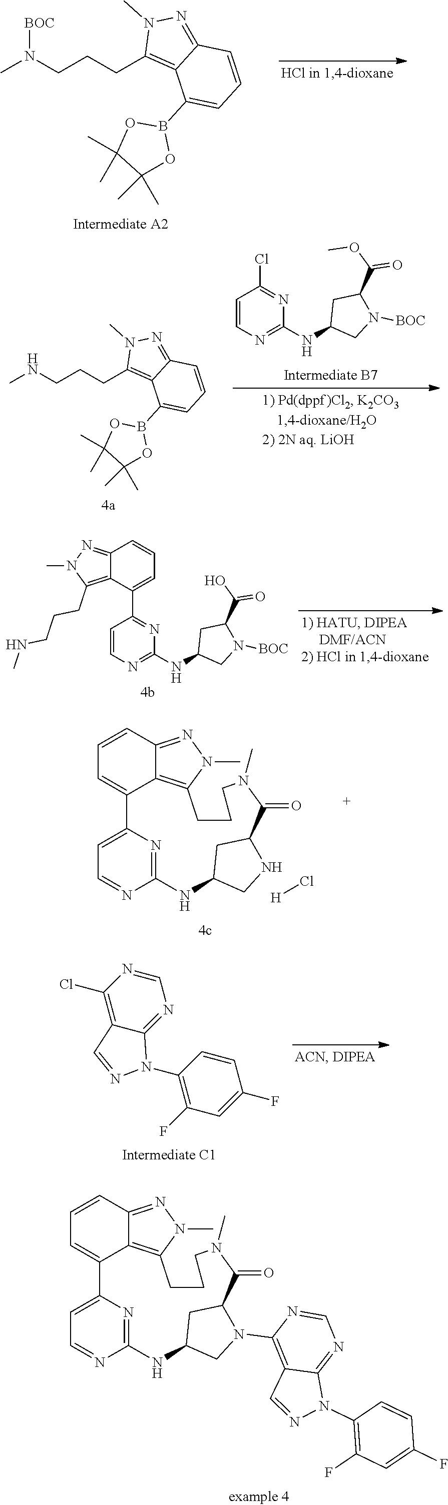

### Step 1: Preparation of N-methyl-3-[2-methyl-4-(4,4,5,5-tetramethyl-1,3,2-dioxaborolan-2-yl)indazol-3-yl]propan-1-amine (Compound 4a)

A mixture of tert-butyl N-methyl-N-[3-[2-methyl-4-(4,4,5,5-tetramethyl-1,3,2-dioxaborolan-2-yl)indazol-3-yl]propyl]carbamate (Intermediate A2, 100 mg, 0.23 mmol) in 4 M HCl in 1,4-dioxane (3 mL) was stirred at room temperature for 4 hrs, then the reaction mixture was filtered to give compound 4a (80 mg), LCMS (M+H)⁺: 330.

### Step 2: Preparation of (2S,4S)-1-tert-butoxycarbonyl-4-[[4-[2-methyl-3-[3-(methylamino)propyl]indazol-4-yl]pyrimidin-2-yl]amino]pyrrolidine-2-carboxylic acid (Compound 4b)

A mixture of potassium carbonate (2.32 g, 16.8 mmol), N-methyl-3-[2-methyl-4-(4,4,5,5-tetramethyl-1,3,2-dioxaborolan-2-yl)indazol-3-yl]propan-1-amine (compound 4a, 2.77 g, 8.41 mmol), O1-tert-butyl O2-methyl (2S,4S)-4-[(4-chloropyrimidin-2-yl)amino]pyrrolidine-1,2-dicarboxylate (intermediate B7, 2 g, 5.61 mmol) and Pd(dppf)Cl₂ (458 mg, 0.56 mmol) in 1,4-dioxane (50 mL) and water (10 mL) was stirred at 95° C. for 2 hrs. Then the reaction mixture was concentrated under reduced pressure to give some residue. The residue was purified by silica gel to give an intermediate, which was dissolved in THF/MeOH (10 mL, 2:1), then 2 N aq. LiOH (2.39 mL, 4.77 mmol) was added and the resulting mixture was stirred at room temperature for 4 hrs. The reaction mixture was adjusted to pH=5, then concentrated to remove most organic solvent to give crude product, the crude was purified by flash preparation (ACN/TFA in water) to give compound 4b (220 mg), LCMS (M+H)⁺: 510.

### Step 3: Preparation of (8S,11S)-13,18-dimethyl-5,7,10,13,18,19,26-heptazapentacyclo[15.6.1.1^{2,6}.1^{8,11}.0^{20,24}]hexacosa-1(23),2,4,6(26),17(24),19,21-heptaen-12-one;hydrochloride (Compound 4c)

To a mixture of HATU (298 mg, 0.78 mmol) and DIPEA (254 mg, 1.96 mmol) in DMF (10 mL) and ACN (100 mL) was added a solution of (2S,4S)-1-tert-butoxycarbonyl-4-[[4-[2-methyl-3-[3-(methylamino)propyl]indazol-4-yl]pyrimidin-2-yl]amino]pyrrolidine-2-carboxylic acid (compound 4b, 150 mg, 0.28 mmol) in DMF (10 mL) and ACN (100 mL) dropwise. The reaction mixture was stirred at room temperature for 1 hr, then concentrated in vacuo, the residue was purified by flash preparation (ACN/TFA in water) to give a light yellow solid, which was then dissolved in 4 M HCl in 1,4-dioxane (5 mL) and stirred at room temperature for 4 hrs. The reaction mixture was concentrated under reduced pressure to give compound 4c (128 mg), LCMS (M+H)⁺: 392.

### Step 4: Preparation of (8S,11S)-10-[1-(2,4-difluorophenyl)pyrazolo[3,4-d]pyrimidin-4-yl]-13,18-dimethyl-5,7,10,13,18,19,26-heptazapentacyclo[15.6.1.1^{2,6}.1^{8,11}.0^{20,24}]hexacosa-1(23),2,4,6(26),17(24),19,21-heptaen-12-one (Example 4)

A mixture of (8S,11S)-13,18-dimethyl-5,7,10,13,18,19,26-heptazapentacyclo[15.6.1.1^{2,6}.1^{8,11}.0^{20,24}]hexacosa-1(23),2,4,6(26),17(24),19,21-heptaen-12-one;hydrochloride (compound 4d, 60 mg, 0.15 mmol), 4-chloro-1-(2,4-difluorophenyl)pyrazolo[3,4-d]pyrimidine (intermediate C1, 49 mg, 0.18 mmol) and DIPEA (99 mg, 0.77 mmol) in ACN (5 mL) was stirred at 80° C. for 2 hrs, then the reaction mixture was concentrated under reduced pressure to give some residue, which was purified by prep-HPLC to give example 4 (28 mg), LCMS (M+H)⁺: 622. ¹H NMR (400 MHz, METHANOL-d₄) δ=8.59-8.32 (m, 2H), 8.27-7.81 (m, 1H), 7.73-7.58 (m, 2H), 7.41-7.11 (m, 4H), 6.96 (d, J=5.1 Hz, 1H), 5.64-5.41 (m, 1H), 5.09-4.96 (m, 1H), 4.57 (dd, J=7.0, 11.6 Hz, 1H), 4.37-4.16 (m, 2H), 4.16-4.09 (m, 3H), 3.79-3.57 (m, 1H), 3.23-3.08 (m, 3H), 3.02-2.82 (m, 2H), 2.80-2.56 (m, 2H), 2.28-1.94 (m, 1H), 1.46-1.28 (m, 1H).

### Example 5

### (8S,11S)-10-[3-(2,4-difluorophenyl)imidazo[1,5-a]pyrazin-8-yl]-13,18-dimethyl-5,7,10,13,18,19,26-heptazapentacyclo[15.6.1.1^{2,6}.1^{8,11}.0^{20,24}]hexacosa-1(23),2,4,6(26),17(24),19,21-heptaen-12-one

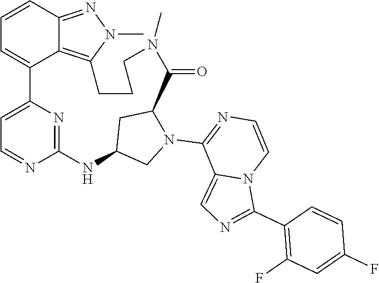 [SMILES: CN1CCCc2c3c(cccc3nn2C)-c2ccnc(n2)N[C@H]2C[C@@H](C1=O)N(c1nccn3c(-c4ccc(F)cc4F)ncc13)C2]

A mixture of (8S,11S)-13,18-dimethyl-5,7,10,13,18,19,26-heptazapentacyclo[15.6.1.1^{2,6}.1^{8,11}.0^{20,24}]hexacosa-1(23),2,4,6(26),17(24),19,21-heptaen-12-one;hydrochloride (compound 4d, 100 mg, 0.26 mmol), 8-chloro-3-(2,4-difluorophenyl)imidazo[1,5-a]pyrazine (intermediate C3, 81 mg, 0.31 mmol), cesium fluoride (194 mg, 1.28 mmol) and DIPEA (165 mg, 1.28 mmol) in DMSO (5 mL) was stirred at 90° C. for 4 hrs. After being cooled to room temperature, the reaction mixture was directly purified by prep-HPLC to give example 5 (10 mg), LCMS (M+H)⁺: 621. ¹H NMR (400 MHz, DMSO-d₆) δ =8.61-8.45 (m, 1H), 8.24-7.99 (m, 1H), 7.81-7.60 (m, 2H), 7.43-7.21 (m, 4H), 7.20-7.01 (m, 2H), 6.97-6.86 (m, 1H), 5.55-5.40 (m, 1H), 5.03-4.86 (m, 1H), 4.15-3.95 (m, 7H), 3.59-3.45 (m, 1H), 3.17-3.05 (m, 3H), 3.01-2.78 (m, 3H), 2.47-2.33 (m, 1H), 2.25-2.12 (m, 1H), 1.42-1.24 (m, 1H).

### Example 6

### (8S,11S)-10-[1-(4-fluoro-2-methoxy-phenyl)pyrazolo[3,4-d]pyrimidin-4-yl]-13,18-dimethyl-5,7,10,13,18,19,26-heptazapentacyclo[15.6.1.1^{2,6}.1^{8,11}.0^{20,24}]hexacosa-1(23),2,4,6(26),17(24),19,21-heptaen-12-one

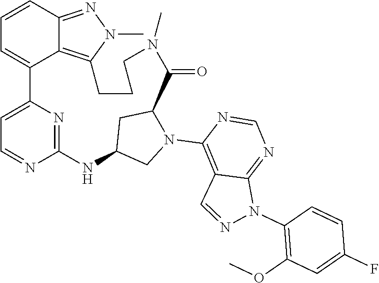 [SMILES: COc1cc(F)ccc1-n1ncc2c(N3C[C@@H]4C[C@H]3C(=O)N(C)CCCc3c5c(cccc5nn3C)-c3ccnc(n3)N4)ncnc21]

The title compound was prepared in analogy to the preparation of example 4 by using intermediate C2 instead of intermediate C1. Example 6 (27 mg), LCMS (M+H)⁺: 634. ¹H NMR (500 MHz, METHANOL-d₄) δ=8.58-8.29 (m, 2H), 8.23-7.78 (m, 1H), 7.69-7.59 (m, 1H), 7.50-7.39 (m, 1H), 7.37-7.28 (m, 1H), 7.24-7.18 (m, 1H), 7.09-7.01 (m, 1H), 7.01-6.93 (m, 1H), 6.92-6.82 (m, 1H), 5.62-5.46 (m, 1H), 5.04-4.99 (m, 1H), 4.63-4.32 (m, 1H), 4.29-4.17 (m, 2H), 4.17-4.09 (m, 3H), 3.80-3.73 (m, 3H), 3.68-3.57 (m, 1H), 3.23-3.09 (m, 3H), 3.03-2.85 (m, 2H), 2.78-2.55 (m, 2H), 2.21-1.94 (m, 1H), 1.44-1.25 (m, 1H).

### Example 7

### (8S,11S)-10-[1-(2,4-difluorophenyl)pyrazolo[3,4-d]pyrimidin-4-yl]-13,18-dimethyl-4,7,10,13,18,19,26-heptazapentacyclo[15.6.1.1^{2,6}.1^{8,11}.0^{20,24}]hexacosa-1(23),2,4,6(26),17(24),19,21-heptaen-12-one

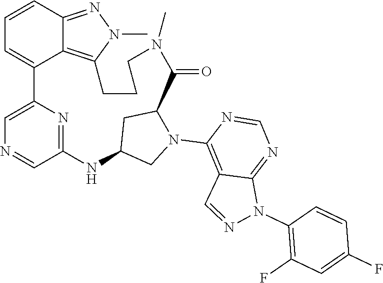 [SMILES: CN1CCCc2c3c(cccc3nn2C)-c2cncc(n2)N[C@H]2C[C@@H](C1=O)N(c1ncnc3c1cnn3-c1ccc(F)cc1F)C2]

The title compound was prepared in analogy to the preparation of example 4 by using intermediate B9 instead of intermediate B7. Example 7 (11 mg), LCMS (M+H)⁺: 622. ¹H NMR (400 MHz, METHANOL-d₄) δ=8.46-8.43 (m, 1H), 8.29-8.13 (m, 1H), 8.10-8.05 (m, 1H), 8.04-7.74 (m, 1H), 7.60-7.54 (m, 1H), 7.53 (dd, J=0.6, 8.7 Hz, 1H), 7.24 (dd, J=7.0, 8.7 Hz, 1H), 7.21-7.16 (m, 1H), 7.14-7.07 (m, 1H), 7.06-7.01 (m, 1H), 5.56-5.42 (m, 1H), 4.87-4.83 (m, 1H), 4.59-4.30 (m, 1H), 4.19 (d, J=11.5 Hz, 1H), 4.12-4.06 (m, 1H), 4.05-4.02 (m, 3H), 3.43-3.34 (m, 1H), 3.13-3.03 (m, 3H), 2.94-2.69 (m, 2H), 2.65-2.55 (m, 1H), 2.41 (d, J=13.4 Hz, 1H), 2.20-2.07 (m, 1H), 1.34-1.22 (m, 1H).

### Example 8

### (8S,11S)-10-[1-(2,4-difluorophenyl)pyrazolo[3,4-d]pyrimidin-4-yl]-13,18-dimethyl-3,7,10,13,18,19,26-heptazapentacyclo[15.6.1.1^{2,6}.1^{8,11}.0^{20,24}]hexacosa-1(23),2,4,6(26),17(24),19,21-heptaen-12-one

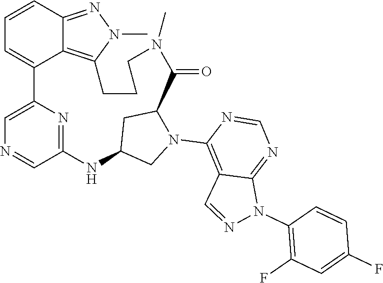 [SMILES: CN1CCCc2c3c(cccc3nn2C)-c2cncc(n2)N[C@H]2C[C@@H](C1=O)N(c1ncnc3c1cnn3-c1ccc(F)cc1F)C2]

The title compound was prepared in analogy to the preparation of example 4 by using intermediate B8 instead of intermediate B7. Example 8 (30 mg), LCMS (M+H)⁺: 622. ¹H NMR (400 MHz, METHANOL-d₄) δ=8.56-8.29 (m, 2H), 8.28-7.84 (m, 1H), 7.74-7.59 (m, 2H), 7.47-7.14 (m, 4H), 6.83-6.70 (m, 1H), 5.69-5.51 (m, 1H), 4.68-4.56 (m, 1H), 4.44-4.11 (m, 6H), 3.90-3.70 (m, 1H), 3.23-3.12 (m, 3H), 2.99-2.83 (m, 2H), 2.82-2.51 (m, 2H), 2.15-1.88 (m, 1H), 1.50-1.26 (m, 1H).

### Example 9

### (8S,11S)-10-[1-(2,4-difluorophenyl)pyrazolo[3,4-d]pyrimidin-4-yl]-13,18-dimethyl-7-oxa-10,13,18,19,26-pentazapentacyclo[15.6.1.1^{2,6}.1^{8,11}.0^{20,24}]hexacosa-1(23),2(26),3,5,17(24),19,21-heptaen-12-one

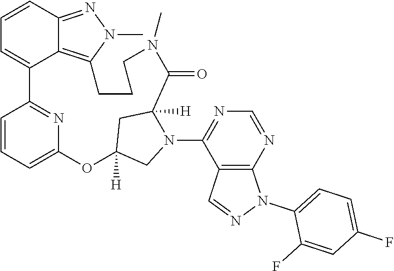 [SMILES: CN1CCCc2c3c(cccc3nn2C)-c2cccc(n2)O[C@H]2C[C@@H](C1=O)N(c1ncnc3c1cnn3-c1ccc(F)cc1F)C2]

The title compound was prepared according to the following scheme:

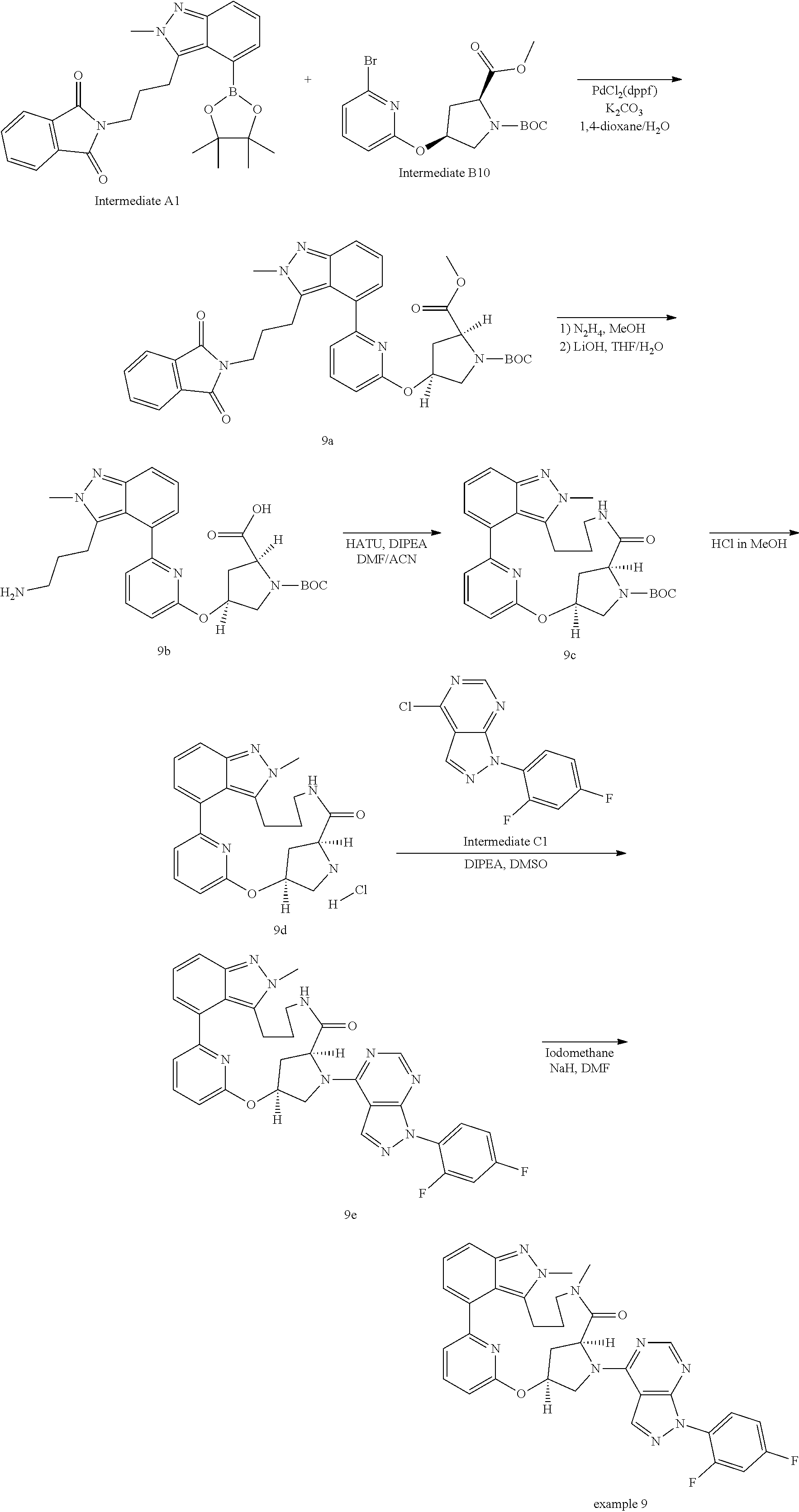

### Step 1: Preparation of O1-tert-butyl O2-methyl (2S,4S)-4-[[6-[3-[3-(1,3-dioxoisoindolin-2-yl)propyl]-2-methyl-indazol-4-yl]-2-pyridyl]oxy]pyrrolidine-1,2-dicarboxylate (Compound 9a)

A mixture of 2-[3-[2-methyl-4-(4,4,5,5-tetramethyl-1,3,2-dioxaborolan-2-yl)indazol-3-yl]propyl]isoindoline-1,3-dione (intermediated A1, 600 mg, 1.35 mmol), O1-tert-butyl 02-methyl (2S,4S)-4-[(6-bromo-2-pyridyl)oxy]pyrrolidine-1,2-dicarboxylate (intermediated B10, 757 mg, 1.89 mmol), Pd(dppf)Cl₂·DCM adduct (49 mg, 0.07 mmol) and potassium carbonate (559 mg, 4.04 mmol) in 1,4-dioxane (10 mL) and water (1 mL) was heated at 100° C. for 1 hr under argon with microwave irritation. After being cooled to room temperature, the reaction mixture was partitioned between water and EtOAc. The separated aqueous layer was extracted with EtOAc for three times, the combined organic layer was washed with water, brine, dried over anhydrous Na₂SO₄, concentrated under reduced to give crude product. The crude was purified by flash column to afford compound 9a (761 mg) as a light yellow foam, LCMS (M+H)⁺: 640.

### Step 2: Preparation of (2S,4S)-4-[[6-[3-(3-aminopropyl)-2-methyl-indazol-4-yl]-2-pyridyl]oxy]-1-tert-butoxycarbonyl-pyrrolidine-2-carboxylic acid (Compound 9b)

A mixture of O1-tert-butyl O2-methyl (2S,4S)-4-[[6-[3-[3-(1,3-dioxoisoindolin-2-yl)propyl]-2-methyl-indazol-4-yl]-2-pyridyl]oxy]pyrrolidine-1,2-dicarboxylate (compound 9a, 761 mg, 1.19 mmol) and hydrazine hydrate (179 mg, 3.57 mmol) in MeOH (20 mL) was heated at 70° C. for 16 hrs in a sealed tube. After being cooled to room temperature, the reaction mixture was diluted with water, then extracted with EtOAc for three times. The combine organic layer was washed with brine, dried over anhydrous Na₂SO₄, concentrated under reduced pressure to give some yellow oil. The yellow oil was dissolved in THF (5 mL) and water (1 mL), then lithium hydroxide monohydrate (190 mg, 4.53 mmol) was added. The resulting mixture was stirred at room temperature for 12 hrs. The reaction mixture was diluted with water, acidified to PH˜5 with 1 N aq. HCl solution, then extracted with EtOAc twice. The combined organic layer was washed with water and brine, dried over anhydrous Na₂SO₄ and concentrated under reduced pressure to afford crude compound 9b (390 mg) as a yellow solid, LCMS (M+H)⁺: 496.

### Step 3: Preparation of tert-butyl (8S,11S)-18-methyl-12-oxo-7-oxa-10,13,18,19,26-pentazapentacyclo[15.6.1.1^{2,6}.1^{8,11}.0^{20,24}]hexacosa-1(23),2(26),3,5,17(24),19,21-heptaene-10-carboxylate (Compound 9c)

To a mixture of DIPEA (326 mg, 2.52 mmol) and HATU (383 mg, 1.01 mmol) in DMF (20 mL) and ACN (20 mL) was added a solution of (2S,4S)-4-[[6-[3-(3-aminopropyl)-2-methyl-indazol-4-yl]-2-pyridyl]oxy]-1-tert-butoxycarbonyl-pyrrolidine-2-carboxylic acid (compound 9b, 250 mg, 0.50 mmol) in DMF (80 mL) and ACN (80 mL) at room temperature dropwise, and then stirred at room temperature for 1 hr. The mixture was concentrated to give some residue, which was diluted with water and EtOAc, then extracted with EtOAc twice. The combined organic layer was washed with water, brine, dried over anhydrous Na₂SO₄, concentrated under reduced pressure to give crude compound 9c (300 mg) as a yellow oil, which was used in next step directly, LCMS (M+H)⁺: 478.

### Step 4: Preparation of (8S,11S)-18-methyl-7-oxa-10,13,18,19,26-pentazapentacyclo[15.6.1.1^{2,6}.1^{8,11}.0^{20,24}]hexacosa-1(23),2(26),3,5,17(24),19,21-heptaen-12-one;hydrochloride (Compound 9d)

A mixture of tert-butyl (8S,11S)-18-methyl-12-oxo-7-oxa-10,13,18,19,26-pentazapentacyclo[15.6.1.1^{2,6}.1^{8,11}.0^{20,24}]hexacosa-1(23),2(26),3,5,17(24),19,21-heptaene-10-carboxylate (compound 9c, 302 mg, 0.25 mmol) and 3 M HCl in MeOH solution (3 mL) was stirred at room temperature for 16 hrs. After being concentrated under reduced pressure, crude compound 9d (371 mg) was obtained as a brown oil, LCMS (M+H)⁺: 378.

### Step 5: Preparation of (8S,11S)-10-[1-(2,4-difluorophenyl)pyrazolo[3,4-d]pyrimidin-4-yl]-18-methyl-7-oxa-10,13,18,19,26-pentazapentacyclo[15.6.1.1^{2,6}.1^{8,11}.0^{20,24}]hexacosa-1(23),2(26),3,5,17(24),19,21-heptaen-12-one (Compound 9e)

A mixture of crude (8S,11S)-18-methyl-7-oxa-10,13,18,19,26-pentazapentacyclo[15.6.1.1^{2,6}.1^{8,11}.0^{20,24}]hexacosa-1(23),2(26),3,5,17(24),19,21-heptaen-12-one;hydrochloride (compound 9d, 370 mg, 0.44 mmol), 4-chloro-1-(2,4-difluorophenyl)pyrazolo[3,4-d]pyrimidine (intermediate C1, 118 mg, 0.44 mmol) and DIPEA (171 mg, 1.32 mmol) in DMSO (15 mL) was heated at 60° C. for 1 hr. After being cooled to room temperature, the reaction mixture was diluted with water, and then extracted with EtOAc twice. The combined organic layer was washed with water, brine, dried over anhydrous Na₂SO₄, concentrated to give crude product, which was purified by flash column to afford crude compound 9e (240 mg) as a yellow oil, LCMS (M+H)⁺: 608.

### Step 6: Preparation of (8S,11S)-10-[1-(2,4-difluorophenyl)pyrazolo[3,4-d]pyrimidin-4-yl]-13,18-dimethyl-7-oxa-10,13,18,19,26-pentazapentacyclo[15.6.1.1^{2,6}.1^{8,11}.0^{20,24}]hexacosa-1(23),2(26),3,5,17(24),19,21-heptaen-12-one (Example 9)

(8S,11S)-10-[1-(2,4-difluorophenyl)pyrazolo[3,4-d]pyrimidin-4-yl]-18-methyl-7-oxa-10,13,18,19,26-pentazapentacyclo[15.6.1.1^{2,6}.1^{8,11}.0^{20,24}]hexacosa-1(23),2(26),3,5,17(24),19,21-heptaen-12-one (compound 9e, 60 mg, 0.04 mmol) was dissolved in anhydrous DMF (1 mL), and then sodium hydride (5 mg, 0.12 mmol) was added at room temperature, 5 mins later, iodomethane (6 mg, 0.04 mmol) was added, the resulting mixture was stirred at room temperature for 12 hrs. The reaction mixture was quenched by MeOH and then directly purified by prep-HPLC to afford example 9 (7 mg) as a white solid, LCMS (M+H)⁺: 622. ¹H NMR (400 MHz, DMSO-d₆) δ=8.68-7.92 (m, 3H), 7.79-7.69 (m, 1H), 7.65-7.55 (m, 2H), 7.36-7.29 (m, 2H), 7.27-7.20 (m, 1H), 7.17-7.06 (m, 2H), 5.69-5.40 (m, 2H), 4.63-4.31 (m, 2H), 4.30-4.00 (m, 4H), 3.86-3.67 (m, 1H), 3.03-2.87 (m, 3H), 2.86-2.42 (m, 4H), 1.85-1.64 (m, 1H), 1.23-1.07 (m, 1H).

### Example 10

### (8S,11S)-10-[1-(2,4-difluorophenyl)pyrazolo[3,4-d]pyrimidin-4-yl]-18-methyl-7-oxa-3,10,13,18,19-pentazapentacyclo[15.6.1.1^{2,6}.1^{8,11}.0^{20,24}]hexacosa-1(23),2(26),3,5,17(24),19,21-heptaen-12-one

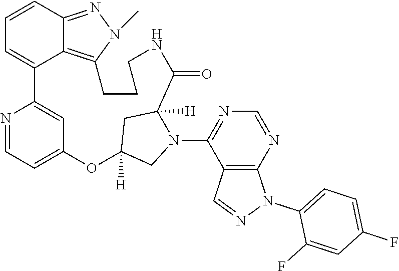 [SMILES: Cn1nc2cccc3c2c1CCCNC(=O)[C@@H]1C[C@@H](CN1c1ncnc2c1cnn2-c1ccc(F)cc1F)Oc1ccnc-3c1]

The title compound was prepared according to the following scheme:

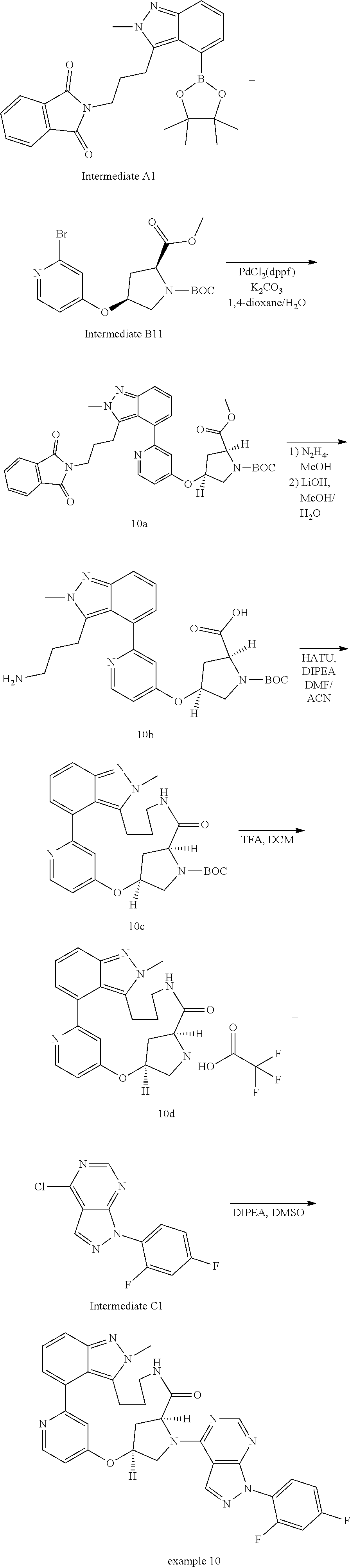

### Step 1: Preparation of O1-tert-butyl O2-methyl (2S,4S)-4-[[2-[3-[3-(1,3-dioxoisoindolin-2-yl)propyl]-2-methyl-indazol-4-yl]-4-pyridyl]oxy]pyrrolidine-1,2-dicarboxylate (Compound 10a)

A mixture of 2-[3-[2-methyl-4-(4,4,5,5-tetramethyl-1,3,2-dioxaborolan-2-yl)indazol-3-yl]propyl]isoindoline-1,3-dione (Intermediate A1, 356 mg, 0.60 mmol), O1-tert-butyl O2-methyl (2S,4S)-4-[(2-bromo-4-pyridyl)oxy]pyrrolidine-1,2-dicarboxylate (intermediated Bit, 361 mg, 0.90 mmol), Pd(dppf)Cl₂·DCM adduct (22 mg, 0.03 mmol) and potassium carbonate (249 mg, 1.8 mmol) in 1,4-dioxane (6 mL) and water (0.6 mL) was heated at 110° C. for 2 hrs under argon with microwave irritation. After being cooled to room temperature, the reaction mixture was partitioned between water and EtOAc. The separated aqueous layer was extracted with EtOAc for three times. The combined organic layer was washed with water, brine, dried over anhydrous Na₂SO₄, concentrated under reduced to give crude product. The crude was purified by flash column to afford compound 10a (164 mg) as a light brown oil, LCMS (M+H)⁺: 640.

### Step 2: Preparation of (2S,4S)-4-[[2-[3-(3-aminopropyl)-2-methyl-indazol-4-yl]-4-pyridyl]oxy]-1-tert-butoxycarbonyl-pyrrolidine-2-carboxylic acid (Compound 10b)

A mixture of O1-tert-butyl O2-methyl (2S,4S)-4-[[2-[3-[3-(1,3-dioxoisoindolin-2-yl)propyl]-2-methyl-indazol-4-yl]-4-pyridyl]oxy]pyrrolidine-1,2-dicarboxylate (compound 10a, 164 mg, 0.19 mmol) and hydrazine hydrate (29 mg, 0.58 mmol) in MeOH (5 mL) was heated at 70° C. for 16 hrs in a sealed tube. After being cooled to room temperature, the reaction mixture was diluted with water, then extracted with EtOAc for three times. The combine organic layer was washed with brine, dried, concentrated under reduced pressure to give some yellow oil. The yellow oil was dissolved in MeOH (2 mL) and water (0.5 mL), then lithium hydroxide monohydrate (48 mg, 1.2 mmol) was added. The resulting mixture was stirred at room temperature for 16 hrs and then directly purified by flash preparation to afford crude compound 10b (99 mg) as a pink solid, LCMS (M+H)⁺: 496.

### Step 3: Preparation of tert-butyl (8S,11S)-18-methyl-12-oxo-7-oxa-3,10,13,18,19-pentazapentacyclo[15.6.1.1^{2,6}.1^{8,11}.0^{20,24}]hexacosa-1(23),2(26),3,5,17(24),19,21-heptaene-10-carboxylate (Compound 10c)

To a mixture of DIPEA (116 mg, 0.9 mmol) and HATU (89 mg, 0.23 mmol) in DMF (4 mL) was added a solution of (2S,4S)-4-[[2-[3-(3-aminopropyl)-2-methyl-indazol-4-yl]-4-pyridyl]oxy]-1-tert-butoxycarbonyl-pyrrolidine-2-carboxylic acid (compound 10b, 99 mg, 0.18 mmol) in DMF (6 mL) and ACN (150 mL) at 0° C. dropwise, and then stirred at 0° C. to room temperature for 16 hrs. The mixture was concentrated to give some residue, which was diluted with water and EtOAc, then extracted with EtOAc for three times. The combined organic layer was washed with water, brine, dried over anhydrous Na₂SO₄, concentrated to give crude compound 10c (73 mg) as a brown oil, which was used in next step directly, LCMS (M+H)⁺: 478.

### Step 4: Preparation of (8S,11S)-18-methyl-7-oxa-3,10,13,18,19-pentazapentacyclo[15.6.1.1^{2,6}.1^{8,11}.0^{20,24}]hexacosa-1(23),2(26),3,5,17(24),19,21-heptaen-12-one;2,2,2-trifluoroacetic acid (Compound 10d)

A mixture of tert-butyl (8S,11S)-18-methyl-12-oxo-7-oxa-3,10,13,18,19-pentazapentacyclo[15.6.1.1^{2,6}.1^{8,11}.0^{20,24}]hexacosa-1(23),2(26),3,5,17(24),19,21-heptaene-10-carboxylate (compound 10c, 73 mg, 0.15 mmol) and TFA (1 mL) in DCM (4 mL) was stirred at room temperature for 3 hrs. After being concentrated under reduced pressure, crude compound 10d (122 mg) was obtained as a brown oil, LCMS (M+H)⁺: 378.

### Step 5: Preparation of (8S,11S)-10-[1-(2,4-difluorophenyl)pyrazolo[3,4-d]pyrimidin-4-yl]-18-methyl-7-oxa-3,10,13,18,19-pentazapentacyclo[15.6.1.1^{2,6}.1^{8,11}.0^{20,24}]hexacosa-1(23),2(26),3,5,17(24),19,21-heptaen-12-one (Example 10)

A mixture of crude (8S,11S)-18-methyl-7-oxa-3,10,13,18,19-pentazapentacyclo[15.6.1.1^{2,6}.1^{8,11}.0^{20,24}]hexacosa-1(23),2(26),3,5,17(24),19,21-heptaen-12-one;2,2,2-trifluoroacetic acid (compound 10d, 122 mg, 0.15 mmol), 4-chloro-1-(2,4-difluorophenyl)pyrazolo[3,4-d]pyrimidine (intermediate C1, 52 mg, 0.19 mmol) and DIPEA (96 mg, 0.75 mmol) in DMSO (2 mL) was heated at 90° C. for 1 hr. After being cooled to room temperature, the reaction mixture was diluted with water and EtOAc, then extracted with EtOAc for three times. The combined organic layer was washed with water, brine, dried over anhydrous Na₂SO₄, concentrated to give crude product. The crude was purified by flash column to afford example 10 (34 mg) as a yellow oil, LCMS (M+H)⁺: 608. ¹H NMR (400 MHz, METHANOL-d₄) δ=8.85 (d, J=6.8 Hz, 1H), 8.59-8.20 (m, 2H), 7.91-7.80 (m, 2H), 7.70-7.57 (m, 2H), 7.47 (dd, J=7.0, 8.7 Hz, 1H), 7.38-7.26 (m, 2H), 7.25-7.17 (m, 1H), 5.93-5.71 (m, 1H), 5.24-5.04 (m, 1H), 4.71-4.29 (m, 2H), 4.19 (s, 3H), 3.84-3.70 (m, 1H), 3.12-3.02 (m, 1H), 3.01-2.72 (m, 4H), 2.08-1.78 (m, 2H).

### Example 11

### (8S,11S)-10-[1-(2,4-difluorophenyl)pyrazolo[3,4-d]pyrimidin-4-yl]-13,18-dimethyl-7-oxa-5,10,13,18,19,26-hexazapentacyclo[15.6.1.1^{2,6}.1^{8,11}.0^{20,24}]hexacosa-1(23),2(26),3,5,17(24),19,21-heptaen-12-one

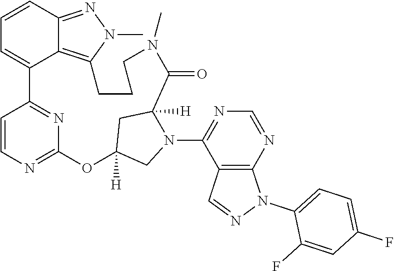 [SMILES: CN1CCCc2c3c(cccc3nn2C)-c2ccnc(n2)O[C@H]2C[C@@H](C1=O)N(c1ncnc3c1cnn3-c1ccc(F)cc1F)C2]

The title compound was prepared in analogy to the preparation of example 9 by using intermediate B12 instead of intermediate B10. Example 11 (85 mg), LCMS (M+H)⁺: 623. ¹H NMR (500 MHz, DMSO-d₆) δ=8.81-8.74 (m, 1H), 8.66-7.91 (m, 2H), 7.79-7.67 (m, 2H), 7.65-7.57 (m, 1H), 7.49 (d, J=5.2 Hz, 1H), 7.36-7.25 (m, 3H), 6.00-5.79 (m, 1H), 5.64-5.52 (m, 1H), 4.60-4.03 (m, 6H), 3.77-3.55 (m, 1H), 3.08-2.90 (m, 3H), 2.88-2.60 (m, 3H), 2.50-2.40 (m, 1H), 1.96-1.76 (m, 1H), 1.28-1.11 (m, 1H).

### Example 12

### (8S,11S)-22-fluoro-10-[1-(4-fluoro-2-methoxy-phenyl)pyrazolo[3,4-d]pyrimidin-4-yl]-13,18-dimethyl-7-oxa-5,10,13,18,19,26-hexazapentacyclo[15.6.1.1^{2,6}.1^{8,11}.0^{20,24}]hexacosa-1(23),2(26),3,5,17(24),19,21-heptaen-12-one

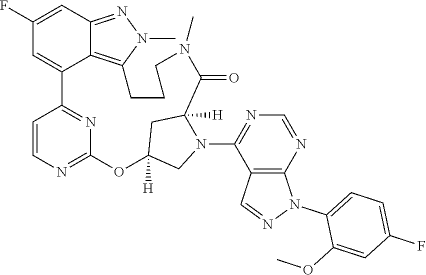 [SMILES: COc1cc(F)ccc1-n1ncc2c(N3C[C@@H]4C[C@H]3C(=O)N(C)CCCc3c5c(cc(F)cc5nn3C)-c3ccnc(n3)O4)ncnc21]

The title compound was prepared according to the following scheme:

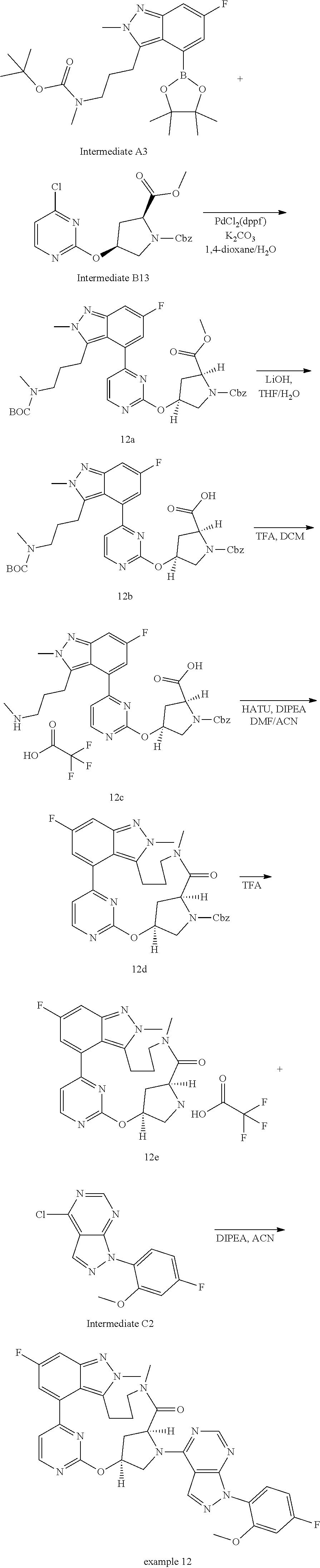

### Step 1: Preparation of O1-benzyl O2-methyl (2S,4S)-4-[4-[3-[3-[tert-butoxycarbonyl(methyl)amino]propyl]-6-fluoro-2-methyl-indazol-4-yl]pyrimidin-2-yl]oxypyrrolidine-1,2-dicarboxylate (Compound 12a)

A mixture of tert-butyl N-[3-[6-fluoro-2-methyl-4-(4,4,5,5-tetramethyl-1,3,2-dioxaborolan-2-yl)indazol-3-yl]propyl]-N-methyl-carbamate (Intermediate A3, 841 mg, 0.9 mmol), O1-benzyl O2-methyl (2S,4S)-4-(4-chloropyrimidin-2-yl)oxypyrrolidine-1,2-dicarboxylate (intermediate B13, 544 mg, 0.9 mmol), Pd(dppf)Cl₂·DCM adduct (37 mg, 0.05 mmol) and potassium carbonate (374 mg, 2.7 mmol) in 1,4-dioxane (10 mL) and water (1 mL) was heated at 90° C. for 15 mins under argon with microwave irritation. After being cooled to room temperature, the reaction mixture was partitioned between water and EtOAc. The separated aqueous layer was extracted with EtOAc twice. The combined organic layer was washed with water, brine, dried over anhydrous Na₂SO₄, concentrated under reduced to give crude product. The crude was purified by flash column to afford compound 12a (920 mg) as a brown oil, LCMS (M+H)⁺: 677.

### Step 2: Preparation of (2S,4S)-1-benzyloxycarbonyl-4-[4-[3-[3-[tert-butoxycarbonyl(methyl)amino]propyl]-6-fluoro-2-methyl-indazol-4-yl]pyrimidin-2-yl]oxy-pyrrolidine-2-carboxylic acid (Compound 12b)

A mixture of O1-benzyl O2-methyl (2S,4S)-4-[4-[3-[3-[tert-butoxycarbonyl(methyl)amino]propyl]-6-fluoro-2-methyl-indazol-4-yl]pyrimidin-2-yl]oxypyrrolidine-1,2-dicarboxylate (compound 12a, 868 mg, 0.83 mmol) and lithium hydroxide monohydrate (245 mg, 5.84 mmol) in THF (6 mL) and water (2 mL) was stirred at room temperature for 16 hrs. The reaction mixture was diluted with water, acidified by 1 N aq. HCl solution, then extracted with EtOAc for three times. The combined organic layer was washed with brine, dried over anhydrous Na₂SO₄, concentrated under reduced pressure to afford compound 12b (692 mg) as a yellow foam, which was directly used in next step without purification, LCMS (M+H)⁺: 663.

### Step 3: Preparation of (2S,4S)-1-benzyloxycarbonyl-4-[4-[6-fluoro-2-methyl-3-[3-(methylamino)propyl]indazol-4-yl]pyrimidin-2-yl]oxy-pyrrolidine-2-carboxylic acid;2,2,2-trifluoroacetic acid (Compound 12c)

A mixture of (2S,4S)-1-benzyloxycarbonyl-4-[4-[3-[3-[tert-butoxycarbonyl(methyl)amino]propyl]-6-fluoro-2-methyl-indazol-4-yl]pyrimidin-2-yl]oxy-pyrrolidine-2-carboxylic acid (compound 12b, 692 mg, 0.78 mmol) and TFA (2 mL) in DCM (6 mL) was stirred at room temperature for 2 hrs. After being concentrated under reduced pressure, crude compound 12c (1.22 g) was obtained as a brown oil, which was directly used in next step without purification, LCMS (M+H)⁺: 563.

### Step 4: Preparation of benzyl (8S,11S)-22-fluoro-13,18-dimethyl-12-oxo-7-oxa-5,10,13,18,19,26-hexazapentacyclo[15.6.1.1^{2,6}.1^{8,11}.0^{20,24}]hexacosa-1(23),2(26),3,5,17(24),19,21-heptaene-10-carboxylate (Compound 12d)

To a mixture of DIPEA (1.12 g, 8.65 mmol) and HATU (411 mg, 1.08 mmol) in DMF (5 mL) was added a solution of (2S,4S)-1-benzyloxycarbonyl-4-[4-[6-fluoro-2-methyl-3-[3-(methylamino)propyl]indazol-4-yl]pyrimidin-2-yl]oxy-pyrrolidine-2-carboxylic acid;2,2,2-trifluoroacetic acid (compound 12c, 1.22 g, 0.72 mmol) in DMF (25 mL) and ACN (300 mL) at 0° C. dropwise, and then stirred at 0° C. to room temperature for 1 hr. The mixture was concentrated to give some residue, which was diluted with water and EtOAc, then extracted with EtOAc for three times. The combined organic layer was washed with water, brine, dried over anhydrous Na₂SO₄, concentrated to give some residue. The residue was purified by flash column to afford crude compound 12d (532 mg) as a brown oil, which was used in next step directly without further purification, LCMS (M+H)⁺: 545.

### Step 5: Preparation of (8S,11S)-22-fluoro-13,18-dimethyl-7-oxa-5,10,13,18,19,26-hexazapentacyclo[15.6.1.1^{2,6}.1^{8,11}.0^{20,24}]hexacosa-1(23),2(26),3,5,17(24),19,21-heptaen-12-one;2,2,2-trifluoroacetic acid (Compound 12e)

A mixture of benzyl (8S,11S)-22-fluoro-13,18-dimethyl-12-oxo-7-oxa-5,10,13,18,19,26-hexazapentacyclo[15.6.1.1^{2,6}.1^{8,11}.0^{20,24}]hexacosa-1(23),2(26),3,5,17(24),19,21-heptaene-10-carboxylate (compound 12d, 532 mg, 0.68 mmol) and TFA (3 mL) was heated at 80° C. for 10 mins with microwave irritation, then concentrated under reduced pressure, crude compound 12e (928 mg) was obtained as a brown oil, which was directly used in next step without purification, LCMS (M+H)⁺: 411.

### Step 6: Preparation of (8S,11S)-22-fluoro-10-[1-(4-fluoro-2-methoxy-phenyl)pyrazolo[3,4-d]pyrimidin-4-yl]-13,18-dimethyl-7-oxa-5,10,13,18,19,26-hexazapentacyclo[15.6.1.1^{2,6}.1^{8,11}.0^{20,24}]hexacosa-1(23),2(26),3,5,17(24),19,21-heptaen-12-one

### Example 12

A mixture of crude (8S,11S)-22-fluoro-13,18-dimethyl-7-oxa-5,10,13,18,19,26-hexazapentacyclo[15.6.1.1^{2,6}.1^{8,11}.0^{20,24}]hexacosa-1(23),2(26),3,5,17(24),19,21-heptaen-12-one;2,2,2-trifluoroacetic acid (compound 12e, 276 mg, 0.20 mmol), 4-chloro-1-(4-fluoro-2-methoxy-phenyl)pyrazolo[3,4-d]pyrimidine (intermediate C2, 67 mg, 0.24 mmol) and DIPEA (207 mg, 1.6 mmol) in anhydrous ACN (3 mL) was heated at 80° C. for 1 hr. After being cooled to room temperature, the reaction mixture was directly purified by prep-HPLC to afford example 12 (78 mg) as a white solid, LCMS (M+H)⁺: 653. ¹H NMR (500 MHz, METHANOL-d₄) δ=8.75 (d, J=5.0 Hz, 1H), 8.49-7.80 (m, 2H), 7.47-7.37 (m, 2H), 7.29 (dd, J=2.2, 9.2 Hz, 1H), 7.19-7.12 (m, 1H), 7.05 (dd, J=2.6, 10.7 Hz, 1H), 6.91-6.84 (m, 1H), 6.12-5.89 (m, 1H), 5.68-5.47 (m, 1H), 4.61-4.21 (m, 3H), 4.14-4.09 (m, 3H), 3.85-3.68 (m, 4H), 3.16-3.03 (m, 3H), 2.95-2.74 (m, 3H), 2.71-2.62 (m, 1H), 2.12-1.92 (m, 1H), 1.40-1.32 (m, 1H).

### Example 13

### (8S,11S)-10-[1-(2,4-difluorophenyl)pyrazolo[3,4-d]pyrimidin-4-yl]-4-methoxy-13,18-dimethyl-7,10,13,18,19,26-hexazapentacyclo[15.6.1.1^{2,6}.1^{8,11}.0^{20,24}]hexacosa-1(23),2,4,6(26),17(24),19,21-heptaen-12-one

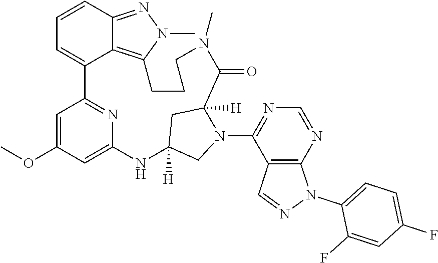 [SMILES: COc1cc2nc(c1)-c1cccc3nn(C)c(c13)CCCN(C)C(=O)[C@@H]1C[C@@H](CN1c1ncnc3c1cnn3-c1ccc(F)cc1F)N2]

The title compound was prepared in analogy to the preparation of example 4 by using intermediate B6 instead of intermediate B7. Example 13 (27.7 mg), LCMS (M+H)⁺: 651. ¹H NMR (400 MHz, METHANOL-d₄) δ=8.55 (s, 1H), 8.27 (s, 1H), 7.78 (d, J=8.3 Hz, 1H), 7.72-7.59 (m, 1H), 7.46-7.38 (m, 1H), 7.34-7.27 (m, 1H), 7.25-7.18 (m, 2H), 6.81-6.67 (m, 2H), 5.70-5.60 (m, 1H), 5.04-4.93 (m, 1H), 5.03-4.93 (m, 1H), 4.76-4.64 (m, 1H), 4.52-4.36 (m, 1H), 4.39-4.26 (m, 1H), 4.18 (s, 6H), 4.11 (d, J=13.6 Hz, 1H), 3.37 (s, 1H), 3.26 (s, 2H), 3.02 (br d, J=12.7 Hz, 1H), 2.82-2.64 (m, 2H), 2.47-2.28 (m, 1H), 1.54 (s, 1H).

### Example 14

### (8S,11S)-10-[1-(4-fluoro-2-methoxy-phenyl)pyrazolo[3,4-d]pyrimidin-4-yl]-4-methoxy-13,18-dimethyl-7,10,13,18,19,26-hexazapentacyclo[15.6.1.1^{2,6}.1^{8,11}.0^{20,24}]hexacosa-1(23),2,4,6(26),17(24),19,21-heptaen-12-one

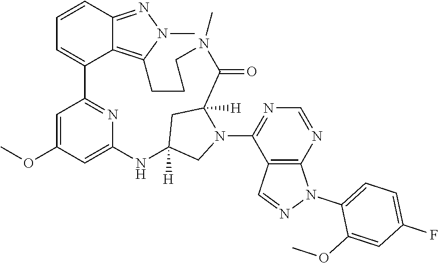 [SMILES: COc1cc2nc(c1)-c1cccc3nn(C)c(c13)CCCN(C)C(=O)[C@@H]1C[C@@H](CN1c1ncnc3c1cnn3-c1ccc(F)cc1OC)N2]

The title compound was prepared in analogy to the preparation of example 4 by using intermediate B6 instead of intermediate B7 and intermediate C2 instead of intermediate C1. Example 14 (28.7 mg) as a white solid, LCMS (M+H)⁺: 663. ¹H NMR (400 MHz, METHANOL-d₄) δ=8.50 (s, 1H), 8.23 (s, 1H), 7.83-7.75 (m, 1H), 7.50-7.36 (m, 2H), 7.22 (d, J=6.8 Hz, 1H), 7.14-7.02 (m, 1H), 6.97-6.86 (m, 1H), 6.83-6.70 (m, 2H), 5.70-5.58 (m, 1H), 5.06-4.92 (m, 2H), 4.76-4.65 (m, 1H), 4.39-4.27 (m, 1H), 4.26-4.11 (m, 6H), 3.77 (s, 3H), 3.37 (s, 1H), 3.26 (s, 2H), 3.05-2.97 (m, 2H), 2.92-2.61 (m, 3H), 2.44-2.38 (m, 1H), 1.65-1.49 (m, 1H).

### Example 15

### (8S,11S)-13-(cyclopropylmethyl)-10-[1-(2,4-difluorophenyl)pyrazolo[3,4-d]pyrimidin-4-yl]-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one

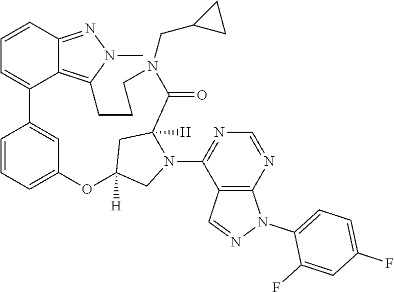 [SMILES: Cn1nc2cccc3c2c1CCCN(CC1CC1)C(=O)[C@@H]1C[C@@H](CN1c1ncnc2c1cnn2-c1ccc(F)cc1F)Oc1cccc-3c1]

The title compound was prepared according to the following scheme:

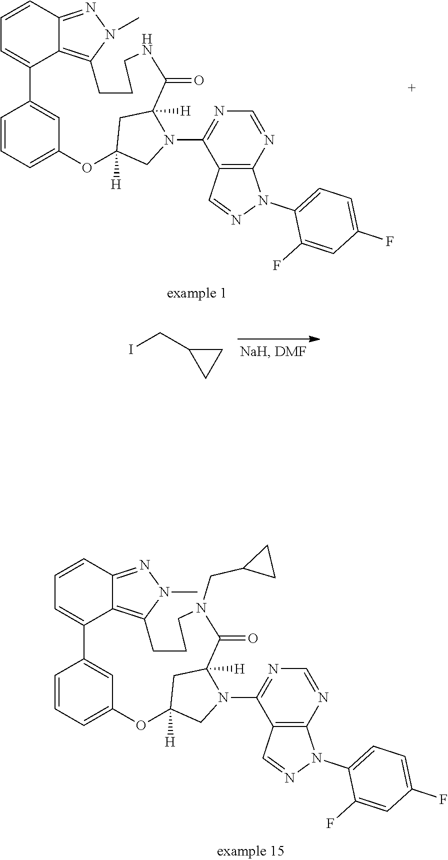 [SMILES: Cn1nc2cccc3c2c1CCCNC(=O)[C@@H]1C[C@@H](CN1c1ncnc2c1cnn2-c1ccc(F)cc1F)Oc1cccc-3c1 ; ICC1CC1 ; Cn1nc2cccc3c2c1CCCN(CC1CC1)C(=O)[C@@H]1C[C@@H](CN1c1ncnc2c1cnn2-c1ccc(F)cc1F)Oc1cccc-3c1]

To a mixture of (8S,11S)-10-[1-(2,4-difluorophenyl)pyrazolo[3,4-d]pyrimidin-4-yl]-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one (example 1, 40 mg, 0.06 mmol) and (iodomethyl)cyclopropane (24 mg, 0.12 mmol) in anhydrous DMF (1 mL) was added sodium hydride (10 mg, 0.24 mmol), the resulting mixture was stirred at room temperature for 16 hrs. The reaction mixture was directly purified by prep-HPLC to afford example 15 (11 mg) as a yellow solid, LCMS (M+H)⁺: 661. ¹H NMR (400 MHz, DMSO-d₆) δ=8.66-7.80 (m, 2H), 7.78-7.70 (m, 1H), 7.64-7.56 (m, 1H), 7.53-7.45 (m, 2H), 7.36-7.30 (m, 1H), 7.28-7.17 (m, 2H), 6.95-6.90 (m, 1H), 6.84-6.74 (m, 2H), 5.66-5.40 (m, 2H), 4.57-4.04 (m, 5H), 3.90-3.80 (m, 1H), 3.22-2.96 (m, 3H), 2.80-2.62 (m, 3H), 2.44-2.04 (m, 2H), 1.61-1.45 (m, 1H), 0.90-0.76 (m, 1H), 0.51-0.31 (m, 4H).

### Example 16

### (8S,11S)-10-[1-(2,4-difluorophenyl)pyrazolo[3,4-d]pyrimidin-4-yl]-18-methyl-13-[(1-methyl-1,2,4-triazol-3-yl)methyl]-7-oxa-10,13,18,19 tetrazapentacyclo[15.6.1.1^{2,6}.1^{8,11}.0^{20,24}]hexacosa-1(23),2(26),3,5,17(24),19,21-heptaen-12-one

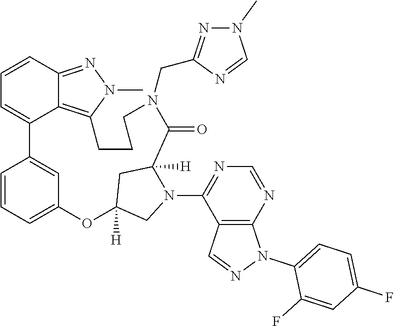 [SMILES: Cn1cnc(CN2CCCc3c4c(cccc4nn3C)-c3cccc(c3)O[C@H]3C[C@@H](C2=O)N(c2ncnc4c2cnn4-c2ccc(F)cc2F)C3)n1]

The title compound was prepared according to the following scheme:

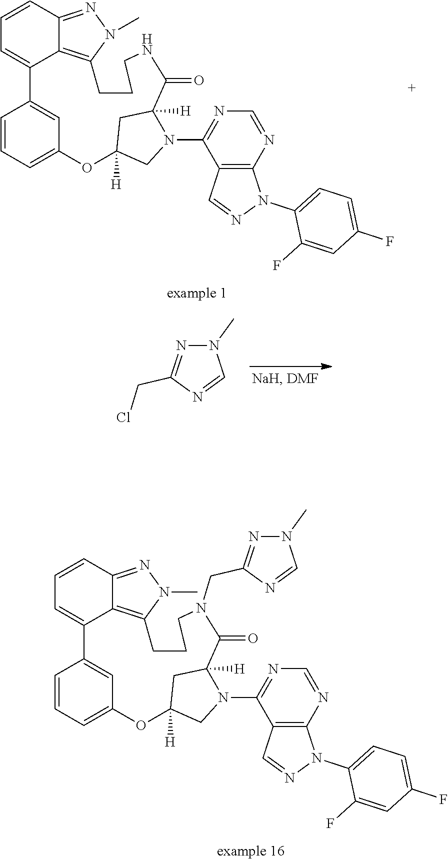

To a mixture of (8S,11S)-10-[1-(2,4-difluorophenyl)pyrazolo[3,4-d]pyrimidin-4-yl]-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one (example 1, 50 mg, 0.08 mmol) and 3-(chloromethyl)-1-methyl-1H-1,2,4-triazole (22 mg, 0.16 mmol) in anhydrous DMF (1 mL) was added sodium hydride (12 mg, 0.25 mmol), the resulting mixture was stirred at room temperature for 16 hrs. The reaction mixture was directly purified by prep-HPLC to afford example 16 (10 mg) as a white solid, LCMS (M+H)⁺: 702. ¹H NMR (400 MHz, DMSO-d₆) δ=8.66-8.19 (m, 3H), 7.78-7.68 (m, 1H), 7.63-7.55 (m, 1H), 7.55-7.44 (m, 2H), 7.36-7.25 (m, 2H), 7.23-7.15 (m, 1H), 6.93 (br d, J=7.5 Hz, 1H), 6.83-6.71 (m, 2H), 5.84-5.30 (m, 2H), 4.84-4.62 (m, 1H), 4.54-4.38 (m, 1H), 4.37-3.95 (m, 5H), 3.93-3.68 (m, 4H), 3.27-2.81 (m, 3H), 2.64-2.50 (m, 2H), 2.22-1.88 (m, 1H), 1.58-1.42 (m, 1H).

### Example 17

### (8S,11S)-10-[1-(2,4-difluorophenyl)pyrazolo[3,4-d]pyrimidin-4-yl]-18-methyl-13-(thiazol-4-ylmethyl)-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one

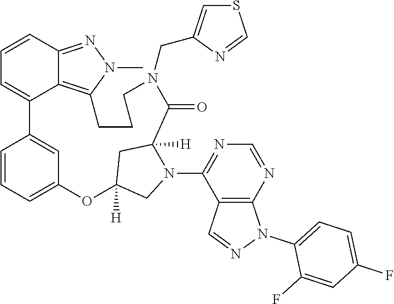 [SMILES: Cn1nc2cccc3c2c1CCCN(Cc1cscn1)C(=O)[C@@H]1C[C@@H](CN1c1ncnc2c1cnn2-c1ccc(F)cc1F)Oc1cccc-3c1]

The title compound was prepared according to the following scheme:

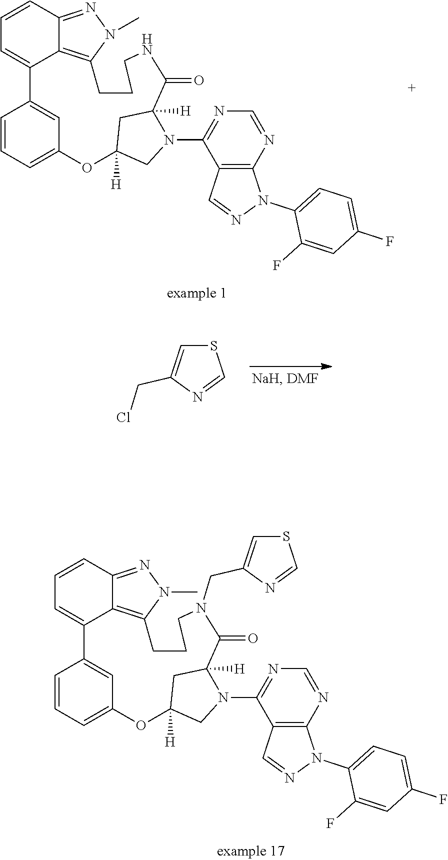 [SMILES: Cn1nc2cccc3c2c1CCCNC(=O)[C@@H]1C[C@@H](CN1c1ncnc2c1cnn2-c1ccc(F)cc1F)Oc1cccc-3c1 ; ClCc1cscn1 ; Cn1nc2cccc3c2c1CCCN(Cc1cscn1)C(=O)[C@@H]1C[C@@H](CN1c1ncnc2c1cnn2-c1ccc(F)cc1F)Oc1cccc-3c1]

To a mixture of (8S,11S)-10-[1-(2,4-difluorophenyl)pyrazolo[3,4-d]pyrimidin-4-yl]-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one (example 1, 40 mg, 0.06 mmol) and 4-(chloromethyl)thiazole (22 mg, 0.13 mmol) in anhydrous DMF (1 mL) was added sodium hydride (10 mg, 0.24 mmol), the resulting mixture was stirred at room temperature for 16 hrs. The reaction mixture was directly purified by prep-HPLC to afford example 17 (17 mg) as a white solid, LCMS (M+H)⁺: 704. ¹H NMR (400 MHz, DMSO-d₆) δ=9.12-8.86 (m, 1H), 8.68-8.22 (m, 2H), 7.80-7.66 (m, 2H), 7.64-7.45 (m, 3H), 7.38-7.26 (m, 2H), 7.25-7.15 (m, 1H), 6.94 (d, J=7.5 Hz, 1H), 6.82-6.73 (m, 2H), 5.72-5.45 (m, 2H), 4.93-4.06 (m, 4H), 4.03-3.66 (m, 4H), 3.13-3.00 (m, 1H), 2.96-2.62 (m, 2H), 2.34-1.92 (m, 3H), 1.54-1.36 (m, 1H).

### Example 18

### (8S,11S)-13-(cyclopropylmethyl)-10-[1-(2,4-difluorophenyl)pyrazolo[3,4-d]pyrimidin-4-yl]-18-methyl-7-oxa-3,5,10,13,18,19-hexazapentacyclo[15.6.1.1^{2,6}.1^{8,11}.0^{20,24}]hexacosa-1(23),2(26),3,5,17(24),19,21-heptaen-12-one

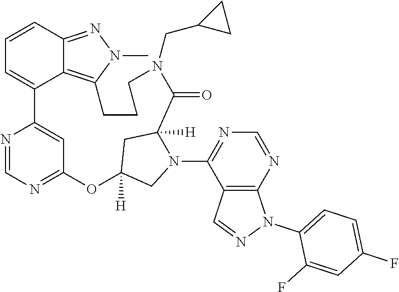 [SMILES: Cn1nc2cccc3c2c1CCCN(CC1CC1)C(=O)[C@@H]1C[C@@H](CN1c1ncnc2c1cnn2-c1ccc(F)cc1F)Oc1cc-3ncn1]

The title compound was prepared in analogy to the preparation of example 9 by using intermediate B14 instead of intermediate B10 and (iodomethyl)cyclopropane instead of iodomethane to give example 18 (5 mg) as a white solid, LCMS (M+H)⁺: 663. ¹H NMR (400 MHz, DMSO-d₆) δ=9.03-8.95 (m, 1H), 8.65-7.82 (m, 2H), 7.78-7.69 (m, 1H), 7.67-7.55 (m, 2H), 7.38-7.26 (m, 2H), 7.20-7.12 (m, 1H), 6.93-6.84 (m, 1H), 6.11-5.88 (m, 1H), 5.58-5.30 (m, 1H), 4.55-4.09 (m, 5H), 3.95-3.85 (m, 1H), 3.50-3.02 (m, 3H), 2.97-2.58 (m, 4H), 2.25-2.04 (m, 1H), 1.67-1.53 (m, 1H), 0.97-0.78 (m, 1H), 0.58-0.30 (m, 4H).

### Example 19

### (8S,11S)-13-(cyclopropylmethyl)-10-[1-(2,4-difluorophenyl)pyrazolo[3,4-d]pyrimidin-4-yl]-18-methyl-7-oxa-5,10,13,18,19,26-hexazapentacyclo[15.6.1.1^{2,6}.1^{8,11}.0^{20,24}]hexacosa-1(23),2(26),3,5,17(24),19,21-heptaen-12-one

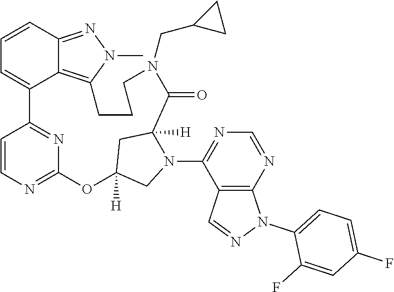 [SMILES: Cn1nc2cccc3c2c1CCCN(CC1CC1)C(=O)[C@@H]1C[C@@H](CN1c1ncnc2c1cnn2-c1ccc(F)cc1F)Oc1nccc-3n1]

The title compound was prepared in analogy to the preparation of example 9 by using intermediate B12 instead of intermediate B10 and (iodomethyl)cyclopropane instead of iodomethane to give example 19 (3 mg) as a white solid, LCMS (M+H)⁺: 663. ¹H NMR (400 MHz, DMSO-d₆) δ=8.79-8.73 (m, 1H), 8.65-7.84 (m, 2H), 7.79-7.66 (m, 2H), 7.64-7.56 (m, 1H), 7.52-7.47 (m, 1H), 7.38-7.26 (m, 3H), 5.94-5.27 (m, 2H), 4.59-4.19 (m, 2H), 4.18-3.89 (m, 5H), 3.88-3.45 (m, 2H), 2.98-2.72 (m, 4H), 1.95-1.38 (m, 2H), 0.51-0.24 (m, 4H), 0.21-0.07 (m, 1H).

### Example 20

### (8S,11S)-13-allyl-10-[1-(2,4-difluorophenyl)pyrazolo[3,4-d]pyrimidin-4-yl]-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one

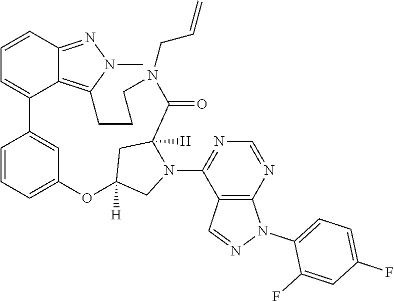 [SMILES: C=CCN1CCCc2c3c(cccc3nn2C)-c2cccc(c2)O[C@H]2C[C@@H](C1=O)N(c1ncnc3c1cnn3-c1ccc(F)cc1F)C2]

The title compound was prepared according to the following scheme:

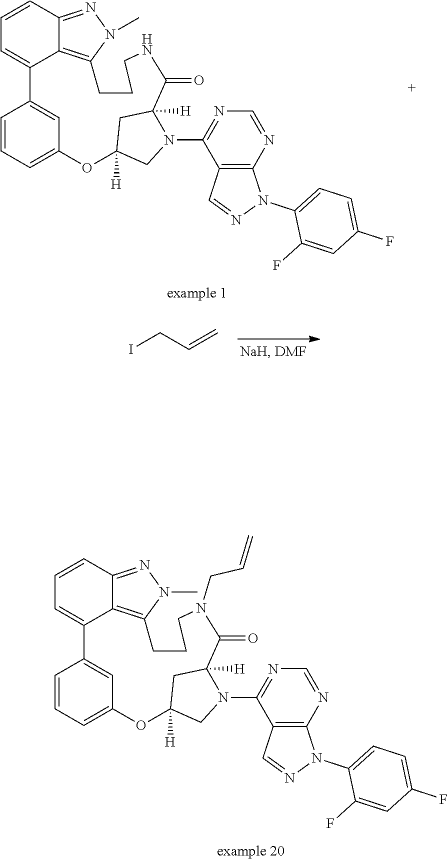

To a mixture of (8S,11S)-10-[1-(2,4-difluorophenyl)pyrazolo[3,4-d]pyrimidin-4-yl]-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one (example 1, 25 mg, 0.04 mmol) and -3-iodoprop-1-ene (13 mg, 0.08 mmol) in anhydrous DMF (1 mL) was added sodium hydride (8 mg, 0.2 mmol), the resulting mixture was stirred at room temperature for 16 hrs. The reaction mixture was directly purified by prep-HPLC to afford example 20 (9 mg) as a white solid, LCMS (M+H)⁺: 647. ¹H NMR (400 MHz, DMSO-d₆) δ=8.67-7.83 (m, 2H), 7.78-7.68 (m, 1H), 7.64-7.56 (m, 1H), 7.53-7.45 (m, 2H), 7.36-7.17 (m, 3H), 6.94 (d, J=7.2 Hz, 1H), 6.85-6.75 (m, 2H), 5.73-5.40 (m, 3H), 5.35-5.21 (m, 2H), 4.56-4.22 (m, 2H), 4.09-3.98 (m, 4H), 3.92-3.78 (m, 2H), 2.92-2.60 (m, 4H), 2.50-2.39 (m, 1H), 2.25-2.04 (m, 1H), 1.58-1.40 (m, 1H).

### Example 21

### (8S,11S)-10-[1-(2,4-difluorophenyl)pyrazolo[3,4-d]pyrimidin-4-yl]-13-(2-methoxyethyl)-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one

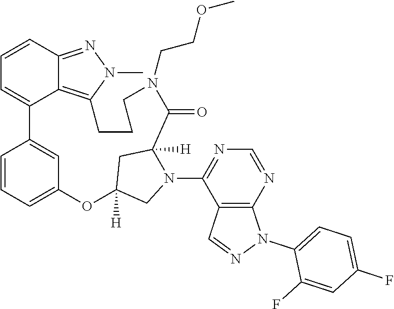 [SMILES: COCCN1CCCc2c3c(cccc3nn2C)-c2cccc(c2)O[C@H]2C[C@@H](C1=O)N(c1ncnc3c1cnn3-c1ccc(F)cc1F)C2]

The title compound was prepared according to the following scheme:

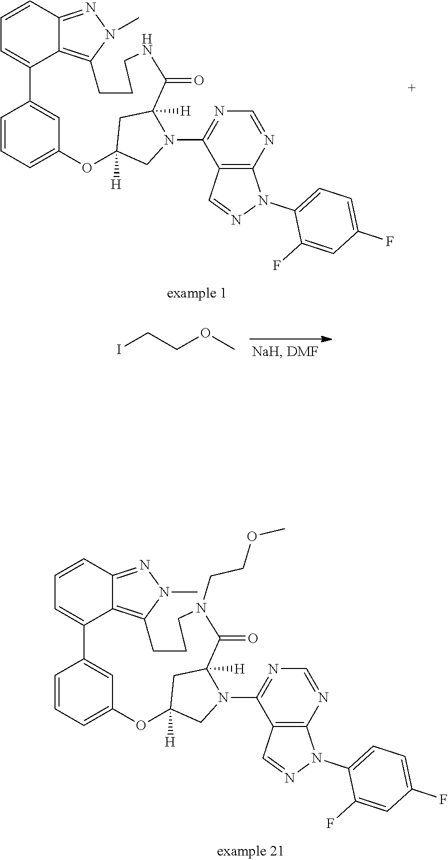

To a mixture of (8S,11S)-10-[1-(2,4-difluorophenyl)pyrazolo[3,4-d]pyrimidin-4-yl]-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one (example 1, 35 mg, 0.06 mmol) and 1-iodo-2-methoxyethane (21 mg, 0.12 mmol) in anhydrous DMF (1 mL) was added sodium hydride (11 mg, 0.3 mmol), the resulting mixture was stirred at room temperature for 16 hrs. The reaction mixture was directly purified by prep-HPLC to afford example 21 (6 mg) as a white solid, LCMS (M+H)⁺: 665. ¹H NMR (400 MHz, DMSO-d₆) δ=8.67-8.32 (m, 1H), 8.28-8.20 (m, 1H), 7.78-7.68 (m, 1H), 7.64-7.54 (m, 1H), 7.53-7.45 (m, 2H), 7.37-7.28 (m, 1H), 7.27-7.17 (m, 2H), 6.97-6.89 (m, 1H), 6.82-6.75 (m, 2H), 5.67-5.28 (m, 2H), 4.54-4.03 (m, 5H), 3.87-3.51 (m, 4H), 3.44-3.32 (m, 2H), 3.27-3.07 (m, 3H), 3.06-2.55 (m, 4H), 2.26-2.10 (m, 1H), 1.58-1.42 (m, 1H).

### Example 22

### (8S,11S)-10-[1-(2,4-difluorophenyl)pyrazolo[3,4-d]pyrimidin-4-yl]-13-(2-hydroxyethyl)-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one

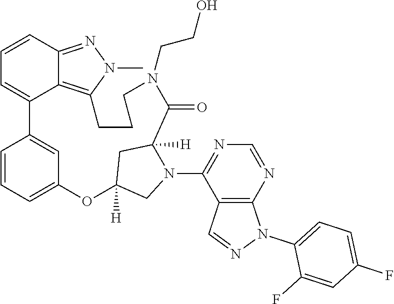 [SMILES: Cn1nc2cccc3c2c1CCCN(CCO)C(=O)[C@@H]1C[C@@H](CN1c1ncnc2c1cnn2-c1ccc(F)cc1F)Oc1cccc-3c1]

The title compound was prepared according to the following scheme:

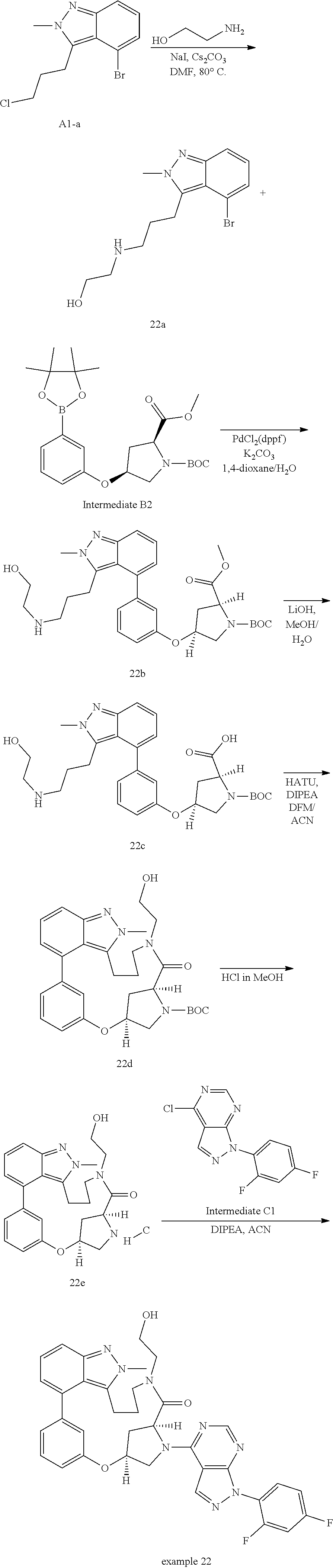

### Step 1: Preparation of 2-[3-(4-bromo-2-methyl-indazol-3-yl)propylamino]ethanol (Compound 22a)

To a solution of 4-bromo-3-(3-chloropropyl)-2-methyl-2H-indazole (Compound A1-a, 2 g, 3.76 mmol), 2-aminoethan-1-ol (0.45 g, 7.51 mmol) and cesium carbonate (3.67 g, 11.3 mmol) in DMF (40 mL) was added sodium iodide (1.13 g, 7.51 mmol), the mixture was stirred at 80° C. for 12 hrs. The mixture was diluted with water and extracted with DCM/MeOH (20:1), the separated organic phase was concentrated under reduced pressure to give crude product, which was purified by flash column to give compound 22a (800 mg) as a colorless oil, LCMS (M+H)⁺: 314.

### Step 2: Preparation of O1-tert-butyl O2-methyl (2S,4S)-4-[3-[3-[3-(2-hydroxyethylamino)propyl]-2-methyl-indazol-4-yl]phenoxy]pyrrolidine-1,2-dicarboxylate (Compound 22b)

A mixture of 2-[3-(4-bromo-2-methyl-indazol-3-yl)propylamino]ethanol (compound 22a, 0.7 g, 2.24 mmol), O1-tert-butyl O2-methyl (2S,4S)-4-[3-(4,4,5,5-tetramethyl-1,3,2-dioxaborolan-2-yl)phenoxy]pyrrolidine-1,2-dicarboxylate (1.2 g, 2.69 mmol), Pd(dppf)C₁₂ (82 mg, 0.11 mmol) and potassium carbonate (0.9 g, 6.73 mmol) in 1,4-dioxane (8 mL) and water (0.8 mL) was heated to 90° C. for 2 hrs under nitrogen with microwave irradiation.

After being cooled to room temperature, the reaction mixture was partitioned between water and DCM. The separated aqueous layer was extracted with DCM and MeOH, the combined organic layer was washed with water, brine, dried over anhydrous Na₂SO₄, concentrated and purified by flash column to give compound 22b (0.8 g) as a brown oil, LCMS (M+H)⁺: 553.

### Step 3: Preparation of (2S,4S)-1-tert-butoxycarbonyl-4-[3-[3-[3-(2-hydroxyethylamino)propyl]-2-methyl-indazol-4-yl]phenoxy]pyrrolidine-2-carboxylic acid (Compound 22c)

A mixture of O1-tert-butyl O2-methyl (2S,4S)-4-[3-[3-[3-(2-hydroxyethylamino)propyl]-2-methyl-indazol-4-yl]phenoxy]pyrrolidine-1,2-dicarboxylate (compound 22b, 0.83 g, 1.2 mmol) and NaOH (240 mg, 6.01 mmol) in MeOH (5 mL) and water (1 mL) was stirred at room temperature for 6 hrs. The reaction mixture was diluted with water and acidified by HOAc to pH˜6, then extracted with EtOAc for three times. The combined organic layer was washed with brine, dried over anhydrous Na₂SO₄, concentrated under reduced pressure to give compound 22c (0.7 g) as a brown solid, LCMS (M+H)⁺: 539.

### Step 4: Preparation of tert-butyl (8S,11S)-13-(2-hydroxyethyl)-18-methyl-12-oxo-7-oxa-10,13,18,19-tetrazapentacyclo[15.6.1.1^{2,6}.1^{8,11}.0^{20,24}]hexacosa-1(23),2(26),3,5,17(24),19,21-heptaene-10-carboxylate (Compound 22d)

To a mixture of HATU (0.98 g, 2.6 mmol) and DIPEA (0.5 g, 3.9 mmol) in DMF (100 mL) was added (2S,4S)-1-tert-butoxycarbonyl-4-[3-[3-[3-(2-hydroxyethylamino)propyl]-2-methyl-indazol-4-yl]phenoxy]pyrrolidine-2-carboxylic acid (compound 22c, 700 mg, 1.3 mmol) in DMF (40 mL) and ACN (980 mL) dropwise at 0° C., the resulting mixture was stirred at 0° C. to room temperature for 16 hrs. The reaction mixture was concentrated to give some residue, which was diluted with water and EtOAc, and then extracted with EtOAc for three times. The combined organic layer was washed with water, brine, dried over anhydrous Na₂SO₄, concentrated under reduced pressure to give crude compound 22d (0.8 g) as a yellow solid, which was directly used in next step without purification, LCMS (M+H)⁺: 521.

### Step 5: preparation (8S,11S)-13-(2-hydroxyethyl)-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;hydrochloride (Compound 22e)

To a mixture of tert-butyl (8S,11S)-13-(2-hydroxyethyl)-18-methyl-12-oxo-7-oxa-10,13,18,19-tetrazapentacyclo[15.6.1.1^{2,6}.1^{8,11}.0^{20,24}]hexacosa-1(23),2(26),3,5,17(24),19,21-heptaene-10-carboxylate (compound 22d, 0.8 g, 1.15 mmol) in MeOH (3 mL) was added a solution of 1 M HCl in MeOH (5.76 mmol), the resulting mixture was stirred at room temperature for 2 hrs. The reaction mixture was concentrated under reduced pressure to give crude compound 22e (520 mg) as a yellow solid, which was directly used in next step without purification, LCMS (M+H)⁺: 421.

### Step 6: Preparation of (8S,11S)-10-[1-(2,4-difluorophenyl)pyrazolo[3,4-d]pyrimidin-4-yl]-13-(2-hydroxyethyl)-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one (Example 22)

A mixture of (8S,11S)-13-(2-hydroxyethyl)-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;hydrochloride (compound 22e, 25 mg, 0.05 mmol), 4-chloro-1-(2,4-difluorophenyl)pyrazolo[3,4-d]pyrimidine (intermediate C1, 21.9 mg, 0.08 mmol) and DIPEA (35.4 mg, 0.27 mmol) in DMSO (1 mL) was heated to 70° C. for 2 hrs. After being cooled to room temperature, the mixture was quenched by MeOH and purified by prep-HPLC to afford example 22 (8 mg) as a white solid, LCMS (M+H)⁺: 651. ¹H NMR (400 MHz, DMSO-d₆) δ=8.67-8.52 (m, 1H), 8.38-8.17 (m, 1H), 7.78-7.67 (m, 1H), 7.64-7.54 (m, 1H), 7.53-7.46 (m, 2H), 7.37-7.17 (m, 3H), 6.93 (t, J=7.5 Hz, 1H), 6.84-6.75 (m, 2H), 5.67-5.53 (m, 1H), 5.50-5.28 (m, 2H), 4.58-4.02 (m, 5H), 3.88-3.53 (m, 3H), 3.50-3.39 (m, 2H), 3.19-2.82 (m, 2H), 2.79-2.57 (m, 4H), 2.24-2.09 (m, 1H), 1.58-1.40 (m, 1H).

### Example 23

### (8S,11S)-10-[1-(2,4-difluorophenyl)pyrazolo[3,4-d]pyrimidin-4-yl]-18-methyl-13-(2-morpholinoethyl)-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one

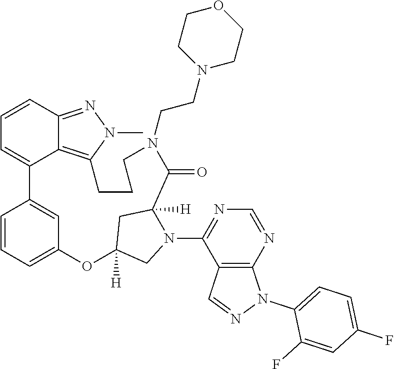 [SMILES: Cn1nc2cccc3c2c1CCCN(CCN1CCOCC1)C(=O)[C@@H]1C[C@@H](CN1c1ncnc2c1cnn2-c1ccc(F)cc1F)Oc1cccc-3c1]

The title compound was prepared according to the following scheme:

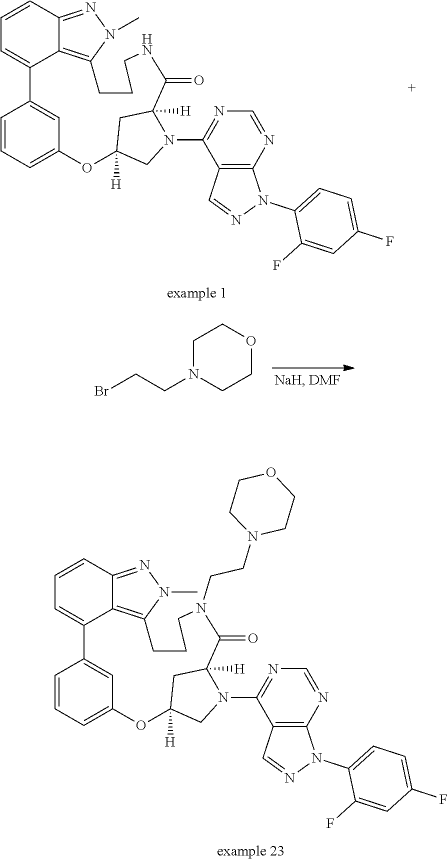 [SMILES: Cn1nc2cccc3c2c1CCCNC(=O)[C@@H]1C[C@@H](CN1c1ncnc2c1cnn2-c1ccc(F)cc1F)Oc1cccc-3c1 ; BrCCN1CCOCC1 ; Cn1nc2cccc3c2c1CCCN(CCN1CCOCC1)C(=O)[C@@H]1C[C@@H](CN1c1ncnc2c1cnn2-c1ccc(F)cc1F)Oc1cccc-3c1]

To a mixture of (8S,11S)-10-[1-(2,4-difluorophenyl)pyrazolo[3,4-d]pyrimidin-4-yl]-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one (example 1, 50 mg, 0.08 mmol) and 4-(2-bromoethyl)morpholine (80 mg, 0.41 mmol) in anhydrous DMF (0.6 mL) was added sodium hydride (10 mg, 0.25 mmol), the resulting mixture was stirred at room temperature for 16 hrs. The reaction mixture was directly purified by prep-HPLC to afford example 23 (20 mg) as a white solid, LCMS (M+H)⁺: 720. ¹H NMR (400 MHz, METHANOL-d₄) δ=8.63-8.21 (m, 2H), 7.73-7.62 (m, 1H), 7.59-7.46 (m, 2H), 7.40-7.27 (m, 2H), 7.26-7.16 (m, 2H), 7.03-6.93 (m, 2H), 6.87 (d, J=6.8 Hz, 1H), 5.75-5.50 (m, 2H), 4.68-4.42 (m, 2H), 4.18-4.11 (m, 3H), 4.08-3.73 (m, 6H), 3.70-3.53 (m, 2H), 3.51-3.15 (m, 5H), 3.07-2.92 (m, 1H), 2.86-2.61 (m, 4H), 2.53-2.36 (m, 1H), 1.67-1.52 (m, 1H).

### Example 24

### (8S,11S)-10-[1-(2,4-difluorophenyl)pyrazolo[3,4-d]pyrimidin-4-yl]-13-[2-(dimethylamino)ethyl]-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one

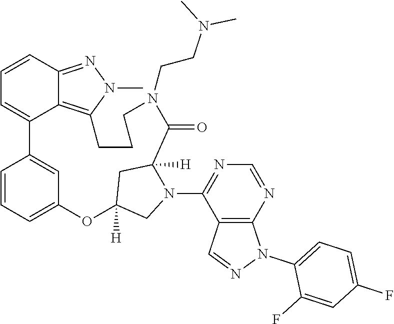 [SMILES: CN(C)CCN1CCCc2c3c(cccc3nn2C)-c2cccc(c2)O[C@H]2C[C@@H](C1=O)N(c1ncnc3c1cnn3-c1ccc(F)cc1F)C2]

The title compound was prepared according to the following scheme:

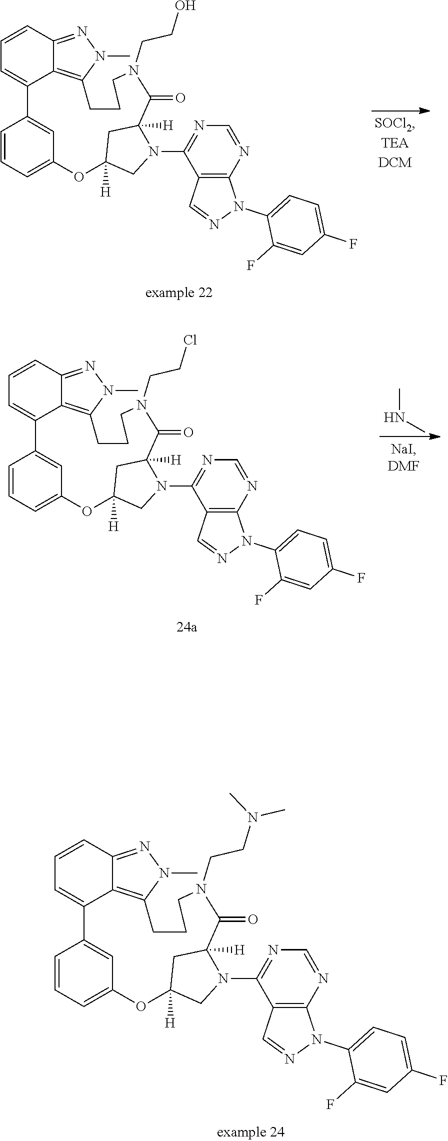

### Step 1: Preparation of (8S,11S)-13-(2-chloroethyl)-10-[1-(2,4-difluorophenyl)pyrazolo[3,4-d]pyrimidin-4-yl]-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one (Compound 24a)

To a solution of (8S,11S)-10-[1-(2,4-difluorophenyl)pyrazolo[3,4-d]pyrimidin-4-yl]-13-(2-hydroxyethyl)-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one (example 22, 50 mg, 0.08 mmol) and triethylamine (23 mg, 0.23 mmol) in DCM (3 mL) was added thionyl chloride (19 mg, 0.16 mmol) at 0° C. under nitrogen, the resulting mixture was stirred at room temperature for 16 hrs. The mixture was added into water and extracted with EtOAc for three times, the combined organic layer was washed with brine, dried over with anhydrous Na₂SO₄, concentrated under reduced pressure to give crude compound 24a (50 mg) as a brown solid, LCMS (M+H)⁺: 683.

### Step 2: Preparation of (8S,11S)-10-[1-(2,4-difluorophenyl)pyrazolo[3,4-d]pyrimidin-4-yl]-13-[2-(dimethylamino)ethyl]-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one (Example 24)

To a solution of (8S,11S)-13-(2-chloroethyl)-10-[1-(2,4-difluorophenyl)pyrazolo[3,4-d]pyrimidin-4-yl]-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one (compound 24a, 50 mg, 0.08 mmol) and sodium iodide (23 mg, 0.15 mmol) in DMF (0.5 mL) was added dimethylamine (7 mg, 0.15 mmol), the resulting mixture was stirred at 80° C. for 2 hrs. After being cooled to room temperature, the mixture was quenched by MeOH and directly purified by prep-HPLC to afford example 24 (13 mg) as a yellow solid, LCMS ((M+H)⁺: 678. ¹H NMR (400 MHz, DMSO-d₆) δ=8.71-7.79 (m, 2H), 7.78-7.69 (m, 1H), 7.64-7.57 (m, 1H), 7.56-7.47 (m, 2H), 7.37-7.18 (m, 3H), 6.95 (d, J=7.6 Hz, 1H), 6.87-6.76 (m, 2H), 5.76-5.29 (m, 2H), 4.62-4.50 (m, 1H), 4.35-4.26 (m, 1H), 4.08 (s, 3H), 3.86-3.74 (m, 2H), 3.36-3.10 (m, 2H), 2.99-2.83 (m, 7H), 2.80-2.59 (m, 4H), 2.43-2.34 (m, 1H), 2.29-2.15 (m, 1H), 1.58-1.42 (m, 1H).

### Example 25

### (8S,11S)-10-[1-(2,4-difluorophenyl)pyrazolo[3,4-d]pyrimidin-4-yl]-22-fluoro-18-methyl-13-(2-morpholinoethyl)-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one

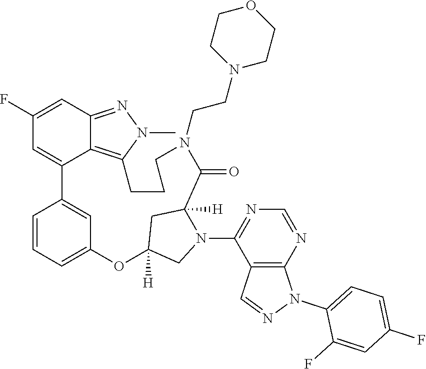 [SMILES: Cn1nc2cc(F)cc3c2c1CCCN(CCN1CCOCC1)C(=O)[C@@H]1C[C@@H](CN1c1ncnc2c1cnn2-c1ccc(F)cc1F)Oc1cccc-3c1]

The title compound was prepared according to the following scheme:

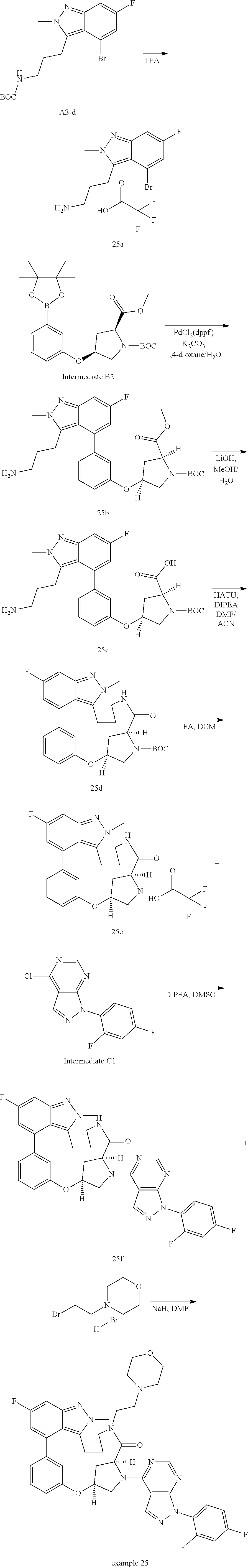

### Step 1: Preparation of 3-(4-bromo-6-fluoro-2-methyl-indazol-3-yl)propan-1-amine;2,2,2-trifluoroacetic acid (Compound 25a)

A solution of tert-butyl N-[3-(4-bromo-6-fluoro-2-methyl-indazol-3-yl)propyl]carbamate (compound A3-d, 1.16 g, 3 mmol) in TFA (5 mL) was stirred at room temperature for 1 hr. After being concentrated under reduced pressure, crude compound 25a (2.74 g) was obtained as a yellow oil, LCMS (M+H)⁺: 286.

### Step 2: Preparation of O1-tert-butyl O2-methyl (2S,4S)-4-[3-[3-(3-aminopropyl)-6-fluoro-2-methyl-indazol-4-yl]phenoxy]pyrrolidine-1,2-dicarboxylate (Compound 25b)

A mixture of 3-(4-bromo-6-fluoro-2-methyl-indazol-3-yl)propan-1-amine;2,2,2-trifluoroacetic acid (compound 25a, 2.74 g, 2.74 mmol), O1-tert-butyl O2-methyl (2S,4S)-4-[3-(4,4,5,5-tetramethyl-1,3,2-dioxaborolan-2-yl)phenoxy]pyrrolidine-1,2-dicarboxylate (intermediate B2, 1.84 g, 4.11 mmol), Pd(dppf)Cl₂·DCM (224 mg, 0.27 mmol) and potassium carbonate (2.27 g, 16.4 mmol) in 1,4-dioxane (15 mL) and water (1.5 mL) was heated at 100° C. for 6 hrs with microwave irritation. After being cooled to room temperature, the reaction mixture was partitioned between water and EtOAc. The separated aqueous layer was extracted with EtOAc for three times, the combined organic layer was washed with brine, dried over anhydrous Na₂SO₄, concentrated under reduced pressure to give crude product, which was purified by flash column to afford compound 25b (924 mg) as a brown oil, LCMS (M+H)⁺: 527.

### Step 3: Preparation of (2S,4S)-4-[3-[3-(3-aminopropyl)-6-fluoro-2-methyl-indazol-4-yl]phenoxy]-1-tert-butoxycarbonyl-pyrrolidine-2-carboxylic acid (Compound 25c)

A mixture of O1-tert-butyl O2-methyl (2S,4S)-4-[3-[3-(3-aminopropyl)-6-fluoro-2-methyl-indazol-4-yl]phenoxy]pyrrolidine-1,2-dicarboxylate (compound 25b, 924 mg, 1.75 mmol) and lithium hydroxide monohydrate (442 mg, 10.53 mmol) in MeOH (6 mL) and water (1.5 mL) was stirred at room temperature for 1 hr. The reaction mixture was diluted with water, acidified by 0.5 N aq. HCl solution, then extracted with DCM for three times. The combined organic layer was washed with brine, dried over anhydrous Na₂SO₄, concentrated under reduced pressure to afford compound 25c (753 mg) as a light brown solid, LCMS (M+H)⁺: 513.

### Step 4: Preparation of tert-butyl (8S,11S)-22-fluoro-18-methyl-12-oxo-7-oxa-10,13,18,19-tetrazapentacyclo[15.6.1.1^{2,6}.1^{8,11}.0^{20,24}]hexacosa-1(23),2(26),3,5,17(24),19,21-heptaene-10-carboxylate (Compound 25d)

To a mixture of DIPEA (749 mg, 7.35 mmol) and HATU (726 mg, 1.91 mmol) in DMF (10 mL) was added a solution of (2S,4S)-4-[3-[3-(3-aminopropyl)-6-fluoro-2-methyl-indazol-4-yl]phenoxy]-1-tert-butoxycarbonyl-pyrrolidine-2-carboxylic acid (compound 25c, 753 mg, 1.47 mmol) in DMF (10 mL) and ACN (400 mL) at 0° C. dropwise and then stirred at 0° C. to room temperature for 16 hrs. The mixture was concentrated to give some residue, which was partitioned between DCM and water, then extracted with DCM for three times. The combined organic layer was washed with brine, concentrated to give crude compound 25d (980 mg) as a brown solid, which was directly used in next step without purification, LCMS (M+H)⁺: 495.

### Step 5: Preparation of (8S,11S)-22-fluoro-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;2,2,2-trifluoroacetic acid (Compound 25e)

A mixture of tert-butyl (8S,11S)-22-fluoro-18-methyl-12-oxo-7-oxa-10,13,18,19-tetrazapentacyclo[15.6.1.1^{2,6}.1^{8,11}.0^{20,24}]hexacosa-1(23),2(26),3,5,17(24),19,21-heptaene-10-carboxylate (compound 25d, 980 mg, 1.39 mmol) and TFA (2 mL) in DCM (4 mL) was stirred at room temperature for 1 hr. After being concentrated under reduced pressure, crude compound 25e (1.87 g) was obtained as a brown oil, which was directly used in next step without purification, LCMS (M+H)⁺: 395.

### Step 6: Preparation of (8S,11S)-10-[1-(2,4-difluorophenyl)pyrazolo[3,4-d]pyrimidin-4-yl]-22-fluoro-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one (Compound 25f)

A mixture of (8S,11S)-22-fluoro-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;2,2,2-trifluoroacetic acid (compound 25e, 1.87 g, 1.29 mmol), 4-chloro-1-(2,4-difluorophenyl)pyrazolo[3,4-d]pyrimidine (intermediate C1, 446 mg, 1.67 mmol) and DIPEA (832 mg, 6.44 mmol) in DMSO (6 mL) was heated at 90° C. for 1 hr. After being cooled to room temperature, the reaction mixture was partitioned between water and DCM. The separated aqueous layer was extracted with DCM for three times, the combined organic layer was washed with brine, dried over anhydrous Na₂SO₄, concentrated in vacuo to give crude product. The crude was purified by flash column to afford compound 25f (510 mg) as a yellow solid, which was directly used in next step without further purification, LCMS (M+H)⁺: 625.

### Step 7: Preparation of (8S,11S)-10-[1-(2,4-difluorophenyl)pyrazolo[3,4-d]pyrimidin-4-yl]-22-fluoro-18-methyl-13-(2-morpholinoethyl)-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one (Example 25)

To a mixture of (8S,11S)-10-[1-(2,4-difluorophenyl)pyrazolo[3,4-d]pyrimidin-4-yl]-22-fluoro-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one (compound 25f, 50 mg, 0.06 mmol) and 4-(2-bromoethyl)morpholine;hydrobromide (48 mg, 0.18 mmol) in anhydrous DMF (0.6 mL) was added sodium hydride (18 mg, 0.46 mmol), the resulting mixture was stirred at room temperature for 16 hrs. The reaction mixture was quenched by MeOH and water, then directly purified by prep-HPLC to afford example 25 (13 mg) as a yellow solid, LCMS (M+H)⁺: 738. ¹H NMR (400 MHz, METHANOL-d₄) δ=8.56-8.14 (m, 2H), 7.71-7.44 (m, 2H), 7.34-7.15 (m, 3H), 7.14-7.05 (m, 1H), 7.02-6.89 (m, 2H), 6.68 (br d, J=9.8 Hz, 1H), 5.72-5.30 (m, 2H), 4.62-4.28 (m, 2H), 4.16-3.93 (m, 4H), 3.79-3.50 (m, 4H), 3.46-3.35 (m, 1H), 3.25-3.16 (m, 2H), 3.11-2.90 (m, 2H), 2.88-2.15 (m, 9H), 1.67-1.50 (m, 1H).

### Example 26

### (8S,11S)-10-[1-(2,4-difluorophenyl)pyrazolo[3,4-d]pyrimidin-4-yl]-13-(3-hydroxypropyl)-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one

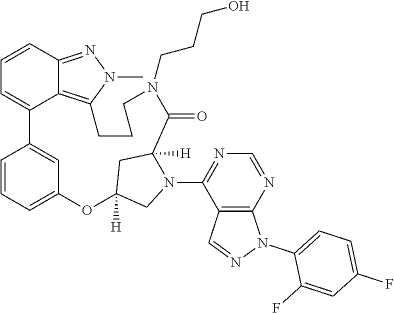 [SMILES: Cn1nc2cccc3c2c1CCCN(CCCO)C(=O)[C@@H]1C[C@@H](CN1c1ncnc2c1cnn2-c1ccc(F)cc1F)Oc1cccc-3c1]

The title compound was prepared in analogy to the preparation of example 12 by using intermediate A5 instead of intermediate A3, intermediate B1 instead of intermediate B13, intermediate C1 instead of intermediate C2 to give example 26 (6 mg) as a white solid, LCMS (M+H)⁺: 665. ¹H NMR (400 MHz, DMSO-d₆) δ=8.67-7.81 (m, 2H), 7.78-7.68 (m, 1H), 7.64-7.55 (m, 1H), 7.54-7.44 (m, 2H), 7.37-7.16 (m, 3H), 6.93 (d, J=7.7 Hz, 1H), 6.84-6.72 (m, 2H), 5.69-5.29 (m, 2H), 4.57-4.44 (m, 1H), 4.33-4.21 (m, 1H), 4.07 (s, 3H), 3.85-3.72 (m, 1H), 3.55-3.35 (m, 3H), 3.22-3.11 (m, 1H), 2.97-2.83 (m, 1H), 2.79-2.57 (m, 3H), 2.48-2.36 (m, 1H), 2.27-2.13 (m, 1H), 1.81-1.68 (m, 1H), 1.63-1.36 (m, 2H).

### Example 27

### (8S,11S)-10-[1-(2,4-difluorophenyl)pyrazolo[3,4-d]pyrimidin-4-yl]-13-(3-methoxypropyl)-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one

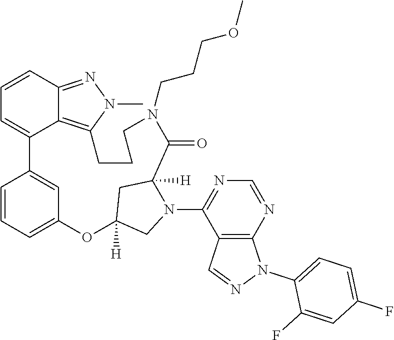 [SMILES: COCCCN1CCCc2c3c(cccc3nn2C)-c2cccc(c2)O[C@H]2C[C@@H](C1=O)N(c1ncnc3c1cnn3-c1ccc(F)cc1F)C2]

The title compound was prepared according to the following scheme:

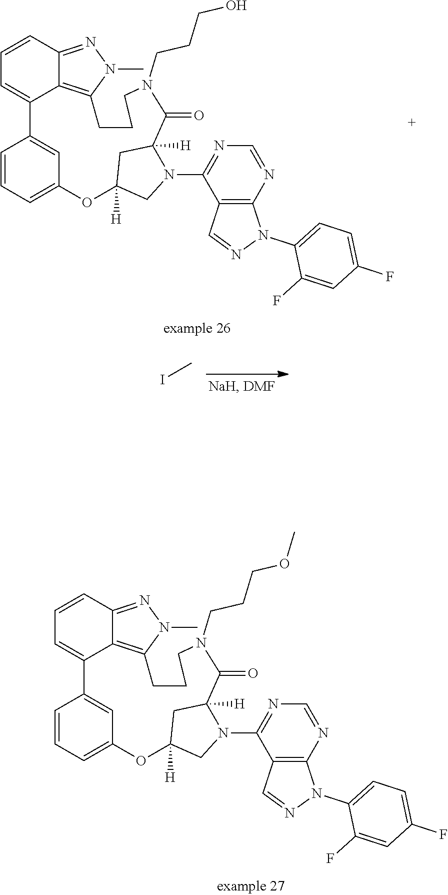

To a mixture of (8S,11S)-10-[1-(2,4-difluorophenyl)pyrazolo[3,4-d]pyrimidin-4-yl]-13-(3-hydroxypropyl)-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one (example 26, 25 mg, 0.04 mmol) and iodomethane (11 mg, 0.04 mmol) in anhydrous DMF (1 mL) was added sodium hydride (5 mg, 0.1 mmol), the resulting mixture was stirred at room temperature for 16 hrs. The reaction mixture was directly purified by prep-HPLC to afford example 27 (6 mg) as a white solid, LCMS (M+H)⁺: 679. ¹H NMR (400 MHz, DMSO-d₆) δ=8.69-7.79 (m, 2H), 7.78-7.69 (m, 1H), 7.65-7.55 (m, 1H), 7.54-7.44 (m, 2H), 7.37-7.16 (m, 3H), 6.93 (d, J=7.6 Hz, 1H), 6.84-6.74 (m, 2H), 5.68-5.28 (m, 2H), 4.57-4.19 (m, 2H), 4.07 (s, 3H), 3.88-3.73 (m, 1H), 3.46-3.29 (m, 3H), 3.28-3.24 (m, 3H), 3.21-3.10 (m, 1H), 2.96-2.84 (m, 1H), 2.80-2.55 (m, 3H), 2.43-2.34 (m, 1H), 2.29-2.13 (m, 1H), 1.91-1.76 (m, 1H), 1.71-1.55 (m, 1H), 1.54-1.39 (m, 1H).

### Example 28

### (8S,11S)-10-[1-(2,4-difluorophenyl)pyrazolo[3,4-d]pyrimidin-4-yl]-18-methyl-13-[2-(4-pyridylmethoxy)ethyl]-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one

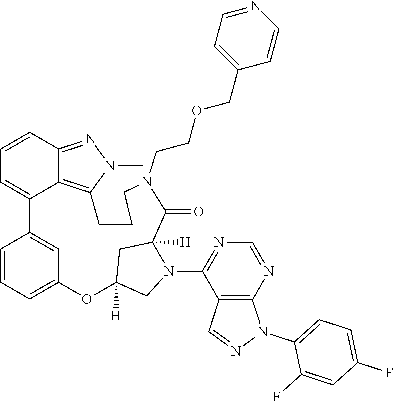 [SMILES: Cn1nc2cccc3c2c1CCCN(CCOCc1ccncc1)C(=O)[C@@H]1C[C@@H](CN1c1ncnc2c1cnn2-c1ccc(F)cc1F)Oc1cccc-3c1]

The title compound was prepared according to the following scheme:

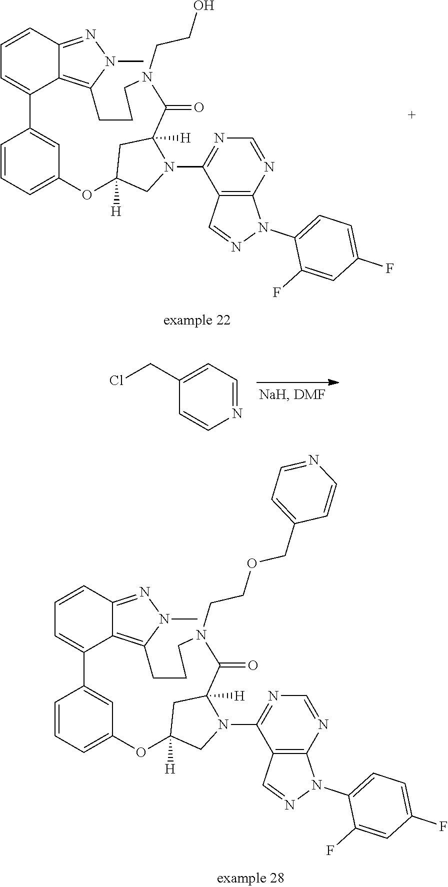 [SMILES: Cn1nc2cccc3c2c1CCCN(CCO)C(=O)[C@@H]1C[C@@H](CN1c1ncnc2c1cnn2-c1ccc(F)cc1F)Oc1cccc-3c1 ; ClCc1ccncc1 ; Cn1nc2cccc3c2c1CCCN(CCOCc1ccncc1)C(=O)[C@@H]1C[C@@H](CN1c1ncnc2c1cnn2-c1ccc(F)cc1F)Oc1cccc-3c1]

To a solution of (8S,11S)-10-[1-(2,4-difluorophenyl)pyrazolo[3,4-d]pyrimidin-4-yl]-13-(2-hydroxyethyl)-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one (example 22, 20 mg, 0.03 mmol) in DMF (0.5 mL) was added sodium hydride (6 mg, 0.15 mmol), the resulting mixture was stirred at room temperature for 5 mins. Then 4-(chloromethyl)pyridine (8 mg, 0.06 mmol) was added and stirred at room temperature for 1 hr. The reaction mixture was directly purified by prep-HPLC to afford example 28 (2 mg) as a white solid, LCMS (M+H)⁺: 742. ¹H NMR (400 MHz, DMSO-d₆) δ=8.67-7.99 (m, 4H), 7.77-7.54 (m, 2H), 7.50 (d, J=8.1 Hz, 1H), 7.43-7.28 (m, 4H), 7.26-7.16 (m, 2H), 6.95-6.87 (m, 1H), 6.85-6.74 (m, 2H), 5.73-5.29 (m, 2H), 4.71-4.45 (m, 3H), 4.36-4.24 (m, 1H), 4.05 (s, 3H), 3.87-3.75 (m, 1H), 3.69-3.41 (m, 3H), 3.08-2.82 (m, 2H), 2.79-2.55 (m, 4H), 2.28-2.11 (m, 1H), 1.58-1.41 (m, 1H).

### Example 29

### (8S,11S)-10-[1-(2,4-difluorophenyl)pyrazolo[3,4-d]pyrimidin-4-yl]-18-methyl-13-(3-phenylpropyl)-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one

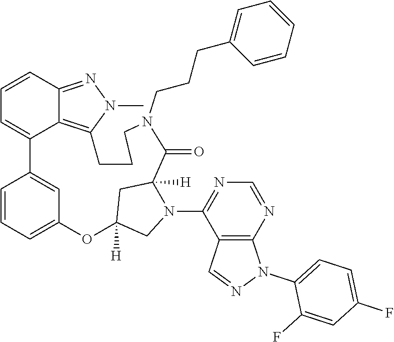 [SMILES: Cn1nc2cccc3c2c1CCCN(CCCc1ccccc1)C(=O)[C@@H]1C[C@@H](CN1c1ncnc2c1cnn2-c1ccc(F)cc1F)Oc1cccc-3c1]

The title compound was prepared according to the following scheme:

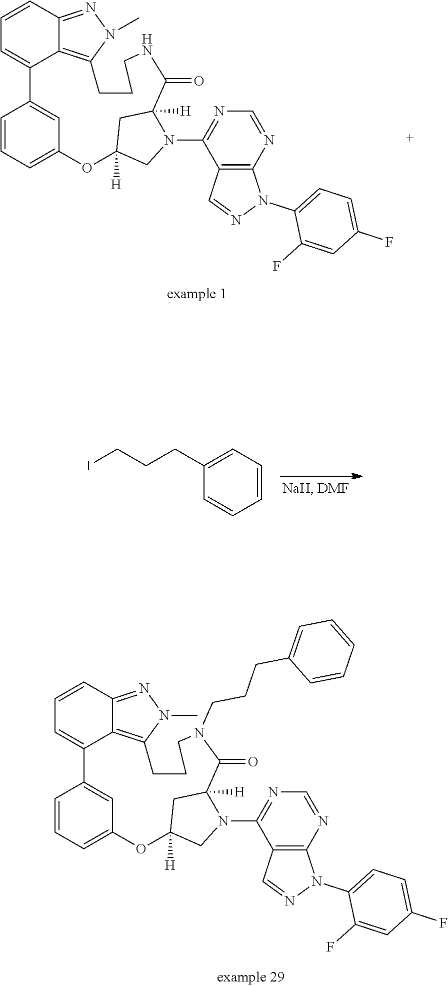 [SMILES: Cn1nc2cccc3c2c1CCCNC(=O)[C@@H]1C[C@@H](CN1c1ncnc2c1cnn2-c1ccc(F)cc1F)Oc1cccc-3c1 ; ICCCc1ccccc1 ; Cn1nc2cccc3c2c1CCCN(CCCc1ccccc1)C(=O)[C@@H]1C[C@@H](CN1c1ncnc2c1cnn2-c1ccc(F)cc1F)Oc1cccc-3c1]

To a mixture of (8S,11S)-10-[1-(2,4-difluorophenyl)pyrazolo[3,4-d]pyrimidin-4-yl]-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one (example 1, 40 mg, 0.07 mmol) and 3-iodopropylbenzene (32 mg, 0.14 mmol) in anhydrous DMF (0.6 mL) was added sodium hydride (11 mg, 0.28 mmol), the resulting mixture was stirred at room temperature for 16 hrs. The reaction mixture was directly purified by prep-HPLC to afford example 29 (13 mg) as a white solid, LCMS (M+H)⁺: 725. ¹H NMR (400 MHz, DMSO-d₆) δ=8.65-7.77 (m, 2H), 7.77-7.68 (m, 1H), 7.64-7.55 (m, 1H), 7.53-7.44 (m, 2H), 7.36-7.28 (m, 3H), 7.25-7.15 (m, 5H), 6.95-6.87 (m, 1H), 6.83-6.72 (m, 2H), 5.61-5.29 (m, 2H), 4.54-4.16 (m, 2H), 4.10-4.03 (m, 3H), 3.90-3.74 (m, 1H), 3.45-3.15 (m, 2H), 2.97-2.81 (m, 1H), 2.75-2.52 (m, 4H), 2.50-2.06 (m, 3H), 1.94-1.82 (m, 1H), 1.69-1.41 (m, 2H).

### Example 30

### (8S,11S)-10-[1-(2,4-difluorophenyl)pyrazolo[3,4-d]pyrimidin-4-yl]-18-methyl-13-(3-morpholinopropyl)-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one

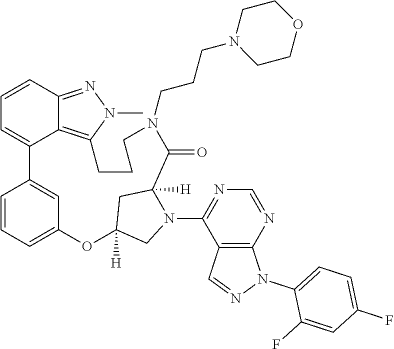 [SMILES: Cn1nc2cccc3c2c1CCCN(CCCN1CCOCC1)C(=O)[C@@H]1C[C@@H](CN1c1ncnc2c1cnn2-c1ccc(F)cc1F)Oc1cccc-3c1]

The title compound was prepared according to the following scheme:

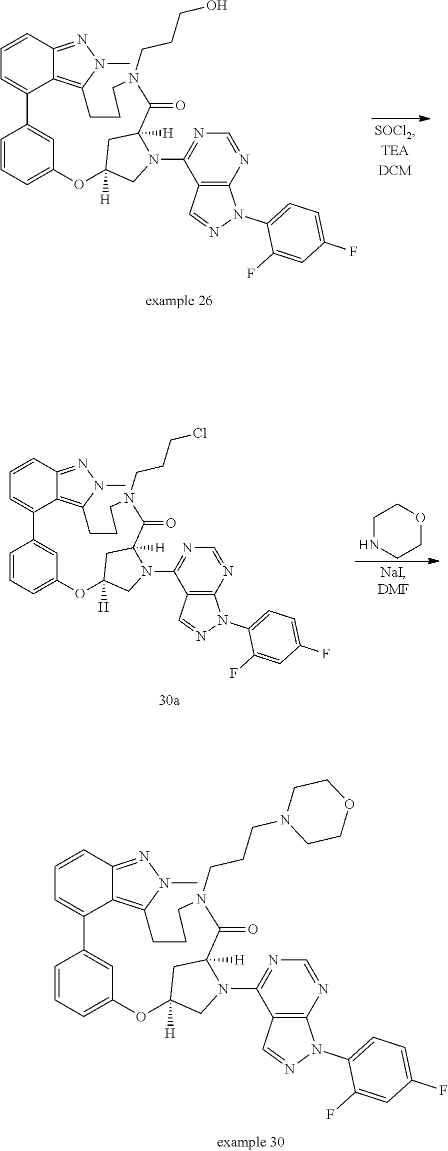 [SMILES: Cn1nc2cccc3c2c1CCCN(CCCO)C(=O)[C@@H]1C[C@@H](CN1c1ncnc2c1cnn2-c1ccc(F)cc1F)Oc1cccc-3c1 ; Cn1nc2cccc3c2c1CCCN(CCCCl)C(=O)[C@@H]1C[C@@H](CN1c1ncnc2c1cnn2-c1ccc(F)cc1F)Oc1cccc-3c1 ; C1COCCN1 ; Cn1nc2cccc3c2c1CCCN(CCCN1CCOCC1)C(=O)[C@@H]1C[C@@H](CN1c1ncnc2c1cnn2-c1ccc(F)cc1F)Oc1cccc-3c1]

### Step 1: Preparation of (8S,11S)-13-(3-chloropropyl)-10-[1-(2,4-difluorophenyl)pyrazolo[3,4-d]pyrimidin-4-yl]-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one (Compound 30a)

To a solution of (8S,11S)-10-[1-(2,4-difluorophenyl)pyrazolo[3,4-d]pyrimidin-4-yl]-13-(3-hydroxypropyl)-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one (example 26, 0.4 g, 0.6 mmol) and TEA (0.2 g, 1.8 mmol) in DCM (10 mL) was added thionyl chloride (0.2 g, 1.2 mmol) at 0° C. under nitrogen. Then the mixture was stirred at room temperature for 16 hrs. The mixture was poured into water and extracted with EtOAc for three times. The combined organic layer was washed with brine, dried over with anhydrous Na₂SO₄, concentrated under reduced pressure to give crude compound 30a (510 mg) as a brown solid, LCMS (M+H)⁺: 683.

### Step 2: Preparation of (8S,11S)-10-[1-(2,4-difluorophenyl)pyrazolo[3,4-d]pyrimidin-4-yl]-18-methyl-13-(3-morpholinopropyl)-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one (Example 30)

To a solution of (8S,11S)-13-(3-chloropropyl)-10-[1-(2,4-difluorophenyl)pyrazolo[3,4-d]pyrimidin-4-yl]-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one (Compound 30a, 40 mg, 0.05 mmol) and sodium iodide (26 mg, 0.15 mmol) in DMF (0.5 mL) was added morpholine (25 mg, 0.25 mmol), the resulting mixture was stirred at 70° C. for 2 hrs. After being cooled to room temperature, the mixture was diluted with MeOH and purified by prep-HPLC to afford example 30 (3 mg) as a white solid, LCMS (M+H)⁺: 734. ¹H NMR (400 MHz, DMSO-d₆) δ=8.65-8.13 (m, 2H), 7.79-7.69 (m, 1H), 7.64-7.56 (m, 1H), 7.53-7.46 (m, 2H), 7.37-7.29 (m, 1H), 7.26 (dd, J=2.0, 8.3 Hz, 1H), 7.23-7.17 (m, 1H), 6.93 (d, J=7.3 Hz, 1H), 6.84-6.72 (m, 2H), 5.66-5.29 (m, 2H), 4.58-4.20 (m, 2H), 4.07 (s, 3H), 3.84-3.73 (m, 1H), 3.65-3.52 (m, 5H), 3.49-3.38 (m, 1H), 3.19-3.09 (m, 1H), 2.97-2.86 (m, 1H), 2.79-2.57 (m, 3H), 2.45-2.16 (m, 7H), 1.79-1.64 (m, 1H), 1.61-1.39 (m, 2H).

### Example 31

### (8S,11S)-10-[1-(2,4-difluorophenyl)pyrazolo[3,4-d]pyrimidin-4-yl]-18-methyl-13-[3-(2-oxa-6-azaspiro[3.3]heptan-6-yl)propyl]-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one

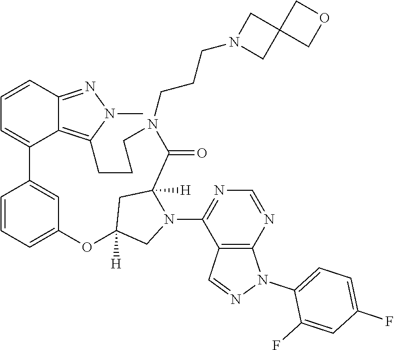 [SMILES: Cn1nc2cccc3c2c1CCCN(CCCN1CC2(COC2)C1)C(=O)[C@@H]1C[C@@H](CN1c1ncnc2c1cnn2-c1ccc(F)cc1F)Oc1cccc-3c1]

The title compound was prepared according to the following scheme:

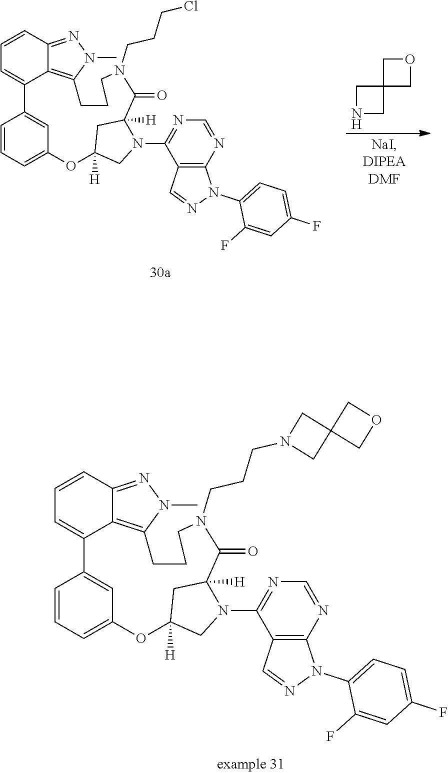 [SMILES: Cn1nc2cccc3c2c1CCCN(CCCCl)C(=O)[C@@H]1C[C@@H](CN1c1ncnc2c1cnn2-c1ccc(F)cc1F)Oc1cccc-3c1 ; C1NCC12COC2 ; Cn1nc2cccc3c2c1CCCN(CCCN1CC2(COC2)C1)C(=O)[C@@H]1C[C@@H](CN1c1ncnc2c1cnn2-c1ccc(F)cc1F)Oc1cccc-3c1]

To a solution of (8S,11S)-13-(3-chloropropyl)-10-[1-(2,4-difluorophenyl)pyrazolo[3,4-d]pyrimidin-4-yl]-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one (compound 30a, 40 mg, 0.05 mmol), sodium iodide (26 mg, 0.15 mmol), DIPEA (23 mg, 0.15 mmol) in DMF (0.5 mL) was added 2-oxa-6-azaspiro[3.3]heptane (12 mg, 0.1 mmol), the resulting mixture was stirred at 70° C. for 2 hrs. After being cooled to room temperature, the mixture was diluted with MeOH and purified by prep-HPLC to afford example 31 (5 mg) as a white solid, LCMS (M+H)⁺: 746. ¹H NMR (400 MHz, DMSO-d₆) δ=8.67-8.11 (m, 2H), 7.79-7.69 (m, 1H), 7.64-7.56 (m, 1H), 7.54-7.45 (m, 2H), 7.37-7.25 (m, 2H), 7.24-7.17 (m, 1H), 6.94 (d, J=7.9 Hz, 1H), 6.83-6.73 (m, 2H), 5.69-5.40 (m, 2H), 4.66-4.48 (m, 4H), 4.37-3.97 (m, 5H), 3.84-3.70 (m, 1H), 3.57-3.38 (m, 2H), 3.25-3.07 (m, 3H), 2.95-2.80 (m, 1H), 2.77-2.55 (m, 3H), 2.47-2.11 (m, 5H), 1.80-1.31 (m, 3H).

### Example 32

### (8S,11S)-10-[1-(2,4-difluorophenyl)pyrazolo[3,4-d]pyrimidin-4-yl]-18-methyl-13-[3-(4-methylpiperazin-1-yl)propyl]-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one

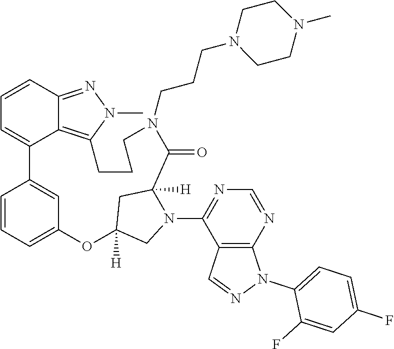 [SMILES: CN1CCN(CCCN2CCCc3c4c(cccc4nn3C)-c3cccc(c3)O[C@H]3C[C@@H](C2=O)N(c2ncnc4c2cnn4-c2ccc(F)cc2F)C3)CC1]

The title compound was prepared according to the following scheme:

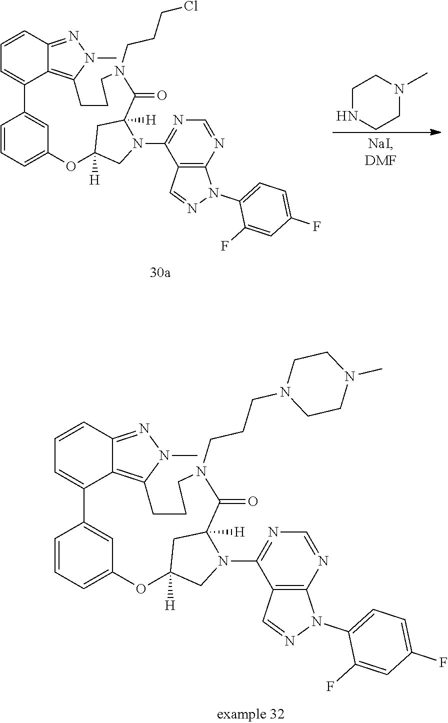

To a solution of (8S,11S)-13-(3-chloropropyl)-10-[1-(2,4-difluorophenyl)pyrazolo[3,4-d]pyrimidin-4-yl]-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one (compound 30a, 40 mg, 0.05 mmol) and sodium iodide (26 mg, 0.15 mmol) in DMF (0.5 mL) was added 1-methylpiperazine (12 mg, 0.1 mmol), the resulting mixture was stirred at 70° C. for 2 hrs. After being cooled to room temperature, the mixture was diluted with MeOH and purified by prep-HPLC to afford example 32 (5 mg) as a white solid, LCMS (M+H)⁺: 747. ¹H NMR (400 MHz, DMSO-d₆) δ=8.66-8.12 (m, 2H), 7.73 (dt, J=6.1, 8.7 Hz, 1H), 7.64-7.56 (m, 1H), 7.54-7.43 (m, 2H), 7.38-7.15 (m, 3H), 6.94 (d, J=7.6 Hz, 1H), 6.86-6.73 (m, 2H), 5.68-5.41 (m, 2H), 4.57-4.25 (m, 2H), 4.07 (s, 3H), 3.85-3.71 (m, 1H), 3.60-3.38 (m, 3H), 3.20-3.07 (m, 2H), 2.96-2.86 (m, 1H), 2.81-2.58 (m, 5H), 2.47-2.35 (m, 4H), 2.33-2.13 (m, 6H), 1.79-1.65 (m, 1H), 1.62-1.43 (m, 2H).

### Example 33

### (8S,11S)-13-[3-(4-acetylpiperazin-1-yl)propyl]-10-[1-(2,4-difluorophenyl)pyrazolo[3,4-d]pyrimidin-4-yl]-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one

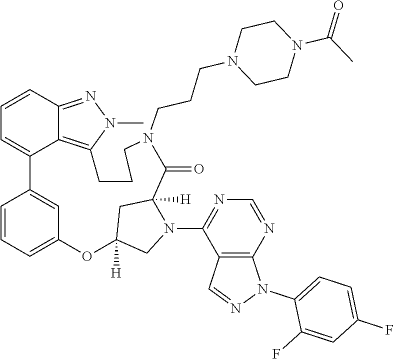 [SMILES: CC(=O)N1CCN(CCCN2CCCc3c4c(cccc4nn3C)-c3cccc(c3)O[C@H]3C[C@@H](C2=O)N(c2ncnc4c2cnn4-c2ccc(F)cc2F)C3)CC1]

The title compound was prepared according to the following scheme:

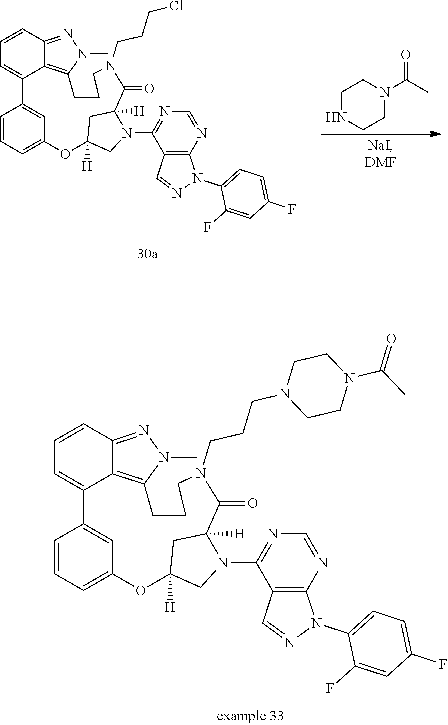

To a solution of (8S,11S)-13-(3-chloropropyl)-10-[1-(2,4-difluorophenyl)pyrazolo[3,4-d]pyrimidin-4-yl]-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one (compound 30a, 40 mg, 0.05 mmol) and sodium iodide (26 mg, 0.15 mmol) in DMF (0.5 mL) was added 1-(piperazin-1-yl)ethan-1-one (15 mg, 0.1 mmol), the resulting mixture was stirred at 80° C. for 2 hrs. After being cooled to room temperature, the mixture was diluted with MeOH and purified by prep-HPLC to afford example 33 (9 mg) as a white solid, LCMS (M+H)⁺: 775. ¹H NMR (400 MHz, DMSO-d₆) δ=8.67-8.09 (m, 2H), 7.78-7.67 (m, 1H), 7.64-7.55 (m, 1H), 7.53-7.46 (m, 2H), 7.37-7.17 (m, 3H), 6.94 (d, J=8.1 Hz, 1H), 6.85-6.73 (m, 2H), 5.68-5.29 (m, 2H), 4.57-4.24 (m, 2H), 4.07 (s, 3H), 3.84-3.73 (m, 1H), 3.49-3.36 (m, 6H), 3.20-3.09 (m, 1H), 2.99-2.82 (m, 1H), 2.80-2.57 (m, 4H), 2.46-2.16 (m, 7H), 1.97 (s, 3H), 1.78-1.67 (m, 1H), 1.61-1.41 (m, 2H).

### Example 34

### (8S,11S)-10-[1-(2,4-difluorophenyl)pyrazolo[3,4-d]pyrimidin-4-yl]-18-methyl-13-[3-(5-methyl-2,5-diazabicyclo[2.2.1]heptan-2-yl)propyl]-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one

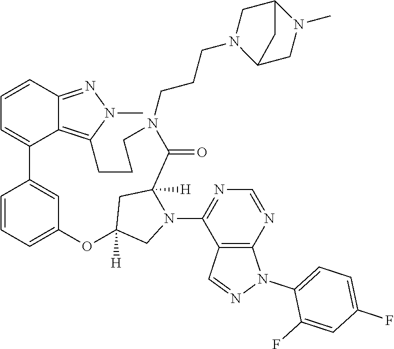 [SMILES: CN1CC2CC1CN2CCCN1CCCc2c3c(cccc3nn2C)-c2cccc(c2)O[C@H]2C[C@@H](C1=O)N(c1ncnc3c1cnn3-c1ccc(F)cc1F)C2]

The title compound was prepared according to the following scheme:

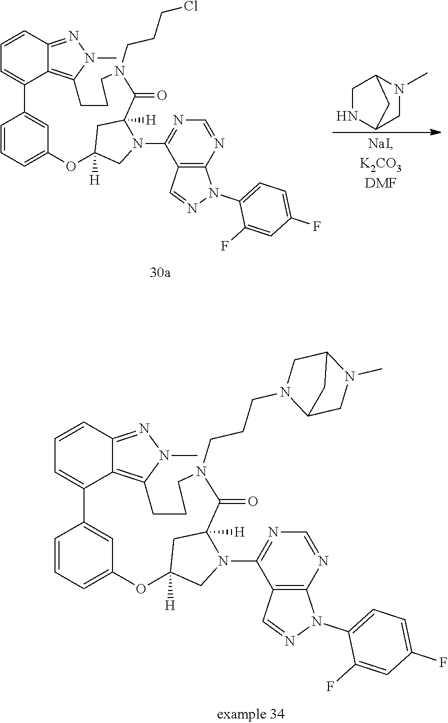

To a solution of (8S,11S)-13-(3-chloropropyl)-10-[1-(2,4-difluorophenyl)pyrazolo[3,4-d]pyrimidin-4-yl]-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one (compound 30a, 40 mg, 0.05 mmol), potassium carbonate (24 mg, 0.15 mmol) and sodium iodide (26 mg, 0.15 mmol) in DMF (0.5 mL) was added 2-methyl-2,5-diazabicyclo[2.2.1]heptane (13 mg, 0.1 mmol), the resulting mixture was stirred at 80° C. for 2 hrs. After being cooled to room temperature, the mixture was diluted with MeOH and purified by prep-HPLC to afford example 34 (4 mg) as a white solid, LCMS (M+H)⁺: 759. ¹H NMR (400 MHz, DMSO-d₆) δ=8.70-8.32 (m, 1H), 8.25-7.76 (m, 1H), 7.74-7.68 (m, 1H), 7.65-7.57 (m, 1H), 7.54-7.46 (m, 2H), 7.33-7.28 (m, 1H), 7.23-7.17 (m, 2H), 6.84-6.75 (m, 2H), 6.70-6.61 (m, 1H), 5.66-5.31 (m, 2H), 4.59-4.47 (m, 1H), 4.32-4.24 (m, 1H), 4.07 (s, 3H), 3.83-3.72 (m, 1H), 3.59-3.42 (m, 2H), 3.27-3.05 (m, 2H), 3.00-2.86 (m, 1H), 2.79-2.53 (m, 9H), 2.46-2.27 (m, 5H), 1.70-1.38 (m, 3H), 1.33-1.27 (m, 2H).

### Example 35

### (8S,11S)-10-[1-(2,4-difluorophenyl)pyrazolo[3,4-d]pyrimidin-4-yl]-18-methyl-13-[3-(4-methylsulfonylpiperazin-1-yl)propyl]-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one

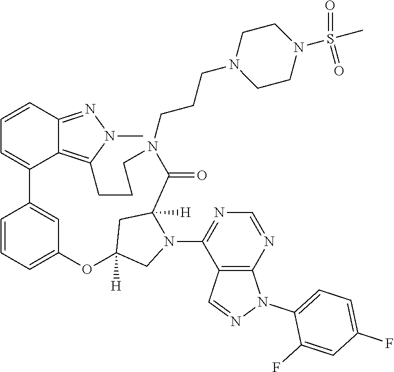 [SMILES: Cn1nc2cccc3c2c1CCCN(CCCN1CCN(S(C)(=O)=O)CC1)C(=O)[C@@H]1C[C@@H](CN1c1ncnc2c1cnn2-c1ccc(F)cc1F)Oc1cccc-3c1]

The title compound was prepared according to the following scheme:

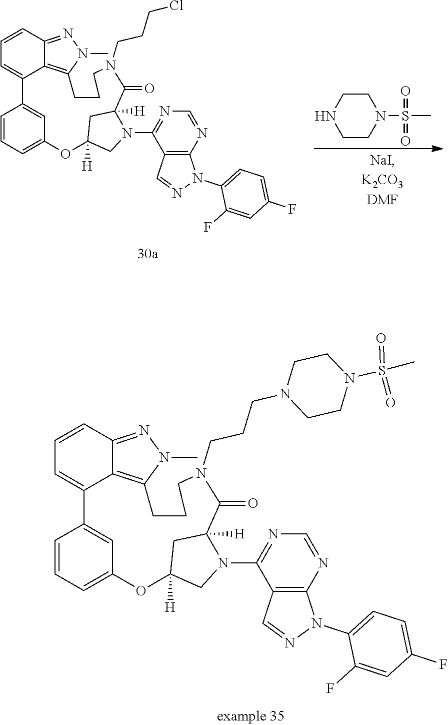

To a solution of (8S,11S)-13-(3-chloropropyl)-10-[1-(2,4-difluorophenyl)pyrazolo[3,4-d]pyrimidin-4-yl]-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one (compound 30a, 40 mg, 0.05 mmol), potassium carbonate (24 mg, 0.15 mmol) and sodium iodide (26 mg, 0.15 mmol) in DMF (0.5 mL) was added 1-(methylsulfonyl)piperazine (30 mg, 0.1 mmol), the resulting mixture was stirred at 80° C. for 2 hrs. After being cooled to room temperature, the mixture was diluted with MeOH and purified by prep-HPLC to afford example (2 mg) as a white solid, LCMS (M+H)⁺: 811. ¹H NMR (400 MHz, DMSO-d₆) δ=8.73-8.10 (m, 2H), 7.81-7.68 (m, 1H), 7.66-7.57 (m, 1H), 7.55-7.46 (m, 2H), 7.38-7.18 (m, 3H), 6.95 (d, J=7.8 Hz, 1H), 6.87-6.74 (m, 2H), 5.72-5.30 (m, 2H), 4.63-4.49 (m, 1H), 4.38-4.25 (m, 1H), 4.08 (s, 3H), 3.87-3.73 (m, 2H), 3.66-3.41 (m, 3H), 3.21-3.00 (m, 8H), 2.97-2.85 (m, 2H), 2.78-2.53 (m, 5H), 2.29-2.14 (m, 2H), 1.81-1.62 (m, 1H), 1.57-1.41 (m, 2H).

### Example 36

### (8S,11S)-10-[1-(2,4-difluorophenyl)pyrazolo[3,4-d]pyrimidin-4-yl]-18-methyl-13-[3-(tetrazol-1-yl)propyl]-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one

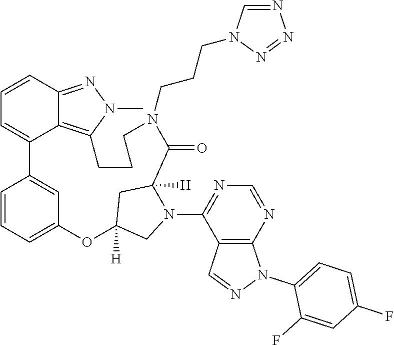 [SMILES: Cn1nc2cccc3c2c1CCCN(CCCn1cnnn1)C(=O)[C@@H]1C[C@@H](CN1c1ncnc2c1cnn2-c1ccc(F)cc1F)Oc1cccc-3c1]

The title compound was prepared according to the following scheme:

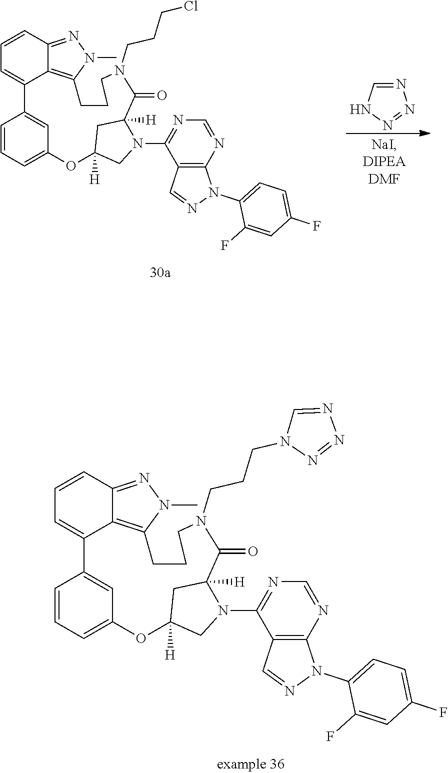 [SMILES: Cn1nc2cccc3c2c1CCCN(CCCCl)C(=O)[C@@H]1C[C@@H](CN1c1ncnc2c1cnn2-c1ccc(F)cc1F)Oc1cccc-3c1 ; c1nnn[nH]1 ; Cn1nc2cccc3c2c1CCCN(CCCn1cnnn1)C(=O)[C@@H]1C[C@@H](CN1c1ncnc2c1cnn2-c1ccc(F)cc1F)Oc1cccc-3c1]

To a solution of (8S,11S)-13-(3-chloropropyl)-10-[1-(2,4-difluorophenyl)pyrazolo[3,4-d]pyrimidin-4-yl]-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one (compound 30a, 40 mg, 0.05 mmol), DIPEA (16 mg, 0.15 mmol) and sodium iodide (26 mg, 0.15 mmol) in DMF (0.5 mL) was added 1H-tetrazole (8 mg, 0.1 mmol), the resulting mixture was stirred at 80° C. for 2 hrs. After being cooled to room temperature, the mixture was diluted with MeOH and purified by prep-HPLC to afford example 36 (3 mg) as a white solid, LCMS (M+H)⁺: 717. ¹H NMR (400 MHz, DMSO-d₆) δ=9.49-9.39 (m, 1H), 8.67-7.78 (m, 2H), 7.77-7.70 (m, 1H), 7.64-7.56 (m, 1H), 7.53-7.45 (m, 2H), 7.37-7.29 (m, 1H), 7.27-7.18 (m, 2H), 6.97-6.87 (m, 1H), 6.83-6.73 (m, 2H), 5.65-5.29 (m, 2H), 4.63-4.49 (m, 3H), 4.28-4.23 (m, 1H), 4.06 (s, 3H), 3.82-3.72 (m, 1H), 3.28-3.15 (m, 1H), 2.96-2.85 (m, 1H), 2.76-2.52 (m, 3H), 2.47-2.12 (m, 3H), 2.08-1.94 (m, 1H), 1.55-1.42 (m, 2H).

### Example 37

### (8S,11S)-10-[1-(2,4-difluorophenyl)pyrazolo[3,4-d]pyrimidin-4-yl]-18-methyl-13-[3-(1,2,4-triazol-1-yl)propyl]-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one

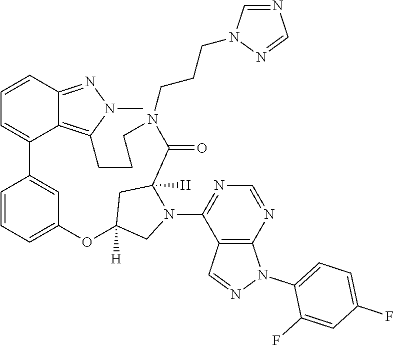 [SMILES: Cn1nc2cccc3c2c1CCCN(CCCn1cncn1)C(=O)[C@@H]1C[C@@H](CN1c1ncnc2c1cnn2-c1ccc(F)cc1F)Oc1cccc-3c1]

The title compound was prepared according to the following scheme:

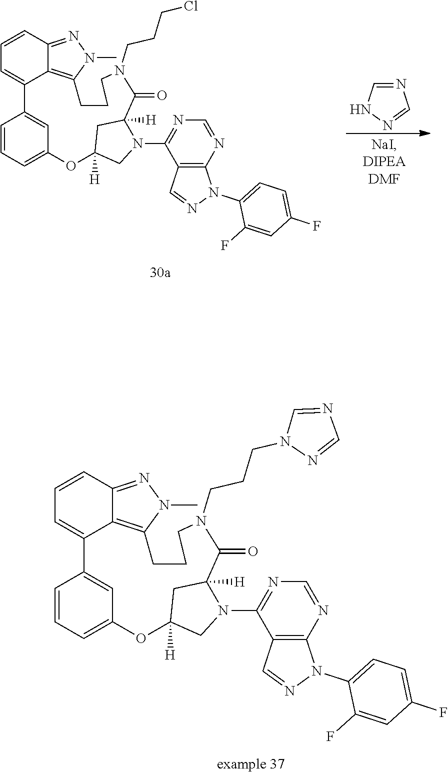 [SMILES: Cn1nc2cccc3c2c1CCCN(CCCCl)C(=O)[C@@H]1C[C@@H](CN1c1ncnc2c1cnn2-c1ccc(F)cc1F)Oc1cccc-3c1 ; c1nc[nH]n1 ; Cn1nc2cccc3c2c1CCCN(CCCn1cncn1)C(=O)[C@@H]1C[C@@H](CN1c1ncnc2c1cnn2-c1ccc(F)cc1F)Oc1cccc-3c1]

To a solution of (8S,11S)-13-(3-chloropropyl)-10-[1-(2,4-difluorophenyl)pyrazolo[3,4-d]pyrimidin-4-yl]-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one (compound 30a, 40 mg, 0.05 mmol), DIPEA (17 mg, 0.15 mmol) and sodium iodide (26 mg, 0.15 mmol) in DMF (0.5 mL) was added 1H-1,2,4-triazole (8 mg, 0.1 mmol), the resulting mixture was stirred at 80° C. for 2 hrs. After being cooled to room temperature, the mixture was diluted with MeOH and purified by prep-HPLC to afford example 37 (1 mg) as a white solid, LCMS (M+H)⁺: 716. ¹H NMR (400 MHz, METHANOL-d₄) δ=8.58-8.53 (m, 2H), 8.25-8.06 (m, 1H), 7.71-7.61 (m, 1H), 7.54-7.46 (m, 2H), 7.33-7.26 (m, 2H), 7.24-7.14 (m, 2H), 6.98-6.93 (m, 2H), 6.85 (d, J=6.8 Hz, 1H), 5.64-5.25 (m, 2H), 4.60-4.53 (m, 1H), 4.47-4.42 (m, 1H), 4.41-4.38 (m, 2H), 4.11 (s, 3H), 4.02-3.95 (m, 1H), 3.60-3.47 (m, 1H), 3.00-2.89 (m, 1H), 2.83-2.51 (m, 4H), 2.46-2.25 (m, 2H), 2.11-1.96 (m, 1H), 1.67-1.55 (m, 2H).

### Example 38

### (8S,11S)-10-[1-(2,4-difluorophenyl)pyrazolo[3,4-d]pyrimidin-4-yl]-13-(3-imidazol-1-ylpropyl)-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one

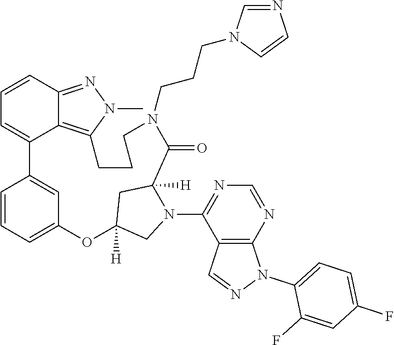 [SMILES: Cn1nc2cccc3c2c1CCCN(CCCn1ccnc1)C(=O)[C@@H]1C[C@@H](CN1c1ncnc2c1cnn2-c1ccc(F)cc1F)Oc1cccc-3c1]

The title compound was prepared according to the following scheme:

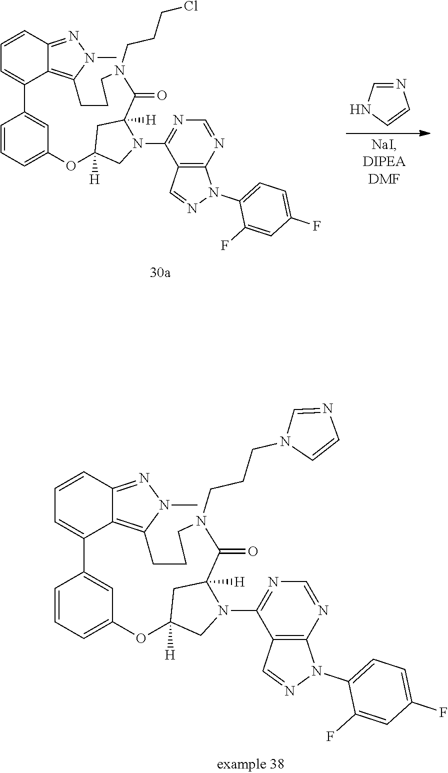 [SMILES: Cn1nc2cccc3c2c1CCCN(CCCCl)C(=O)[C@@H]1C[C@@H](CN1c1ncnc2c1cnn2-c1ccc(F)cc1F)Oc1cccc-3c1 ; c1c[nH]cn1 ; Cn1nc2cccc3c2c1CCCN(CCCn1ccnc1)C(=O)[C@@H]1C[C@@H](CN1c1ncnc2c1cnn2-c1ccc(F)cc1F)Oc1cccc-3c1]

To a solution of (8S,11S)-13-(3-chloropropyl)-10-[1-(2,4-difluorophenyl)pyrazolo[3,4-d]pyrimidin-4-yl]-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one (compound 30a, 40 mg, 0.05 mmol), DIPEA (17 mg, 0.15 mmol) and sodium iodide (26 mg, 0.15 mmol) in DMF (0.5 mL) was added 1H-imidazole (8 mg, 0.1 mmol), the resulting mixture was stirred at 80° C. for 2 hrs. After being cooled to room temperature, the mixture was diluted with MeOH and purified by prep-HPLC to afford example 38 (1.5 mg) as a white solid, LCMS (M+H)⁺: 715. ¹H NMR (400 MHz, DMSO-d₆) δ=8.67-8.12 (m, 2H), 7.79-7.66 (m, 2H), 7.65-7.56 (m, 1H), 7.54-7.45 (m, 2H), 7.38-7.28 (m, 1H), 7.26-7.17 (m, 3H), 6.98-6.88 (m, 2H), 6.82-6.71 (m, 2H), 5.63-5.30 (m, 2H), 4.54-4.19 (m, 2H), 4.12-3.96 (m, 5H), 3.86-3.70 (m, 1H), 3.28-3.07 (m, 1H), 2.91-2.78 (m, 1H), 2.76-2.53 (m, 2H), 2.43-2.31 (m, 1H), 2.25-2.09 (m, 2H), 2.08-1.91 (m, 1H), 1.86-1.71 (m, 1H), 1.55-1.38 (m, 2H).

### Example 39

### (8S,11S)-10-[1-(2,4-difluorophenyl)pyrazolo[3,4-d]pyrimidin-4-yl]-13-[3-(2-methoxyethoxy)propyl]-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one

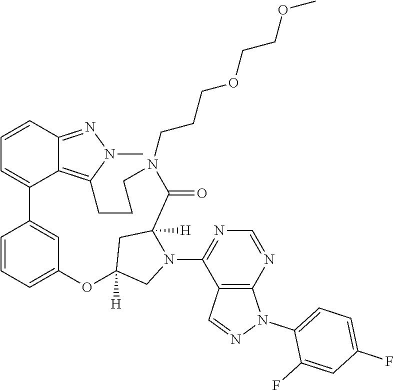 [SMILES: COCCOCCCN1CCCc2c3c(cccc3nn2C)-c2cccc(c2)O[C@H]2C[C@@H](C1=O)N(c1ncnc3c1cnn3-c1ccc(F)cc1F)C2]

The title compound was prepared according to the following scheme:

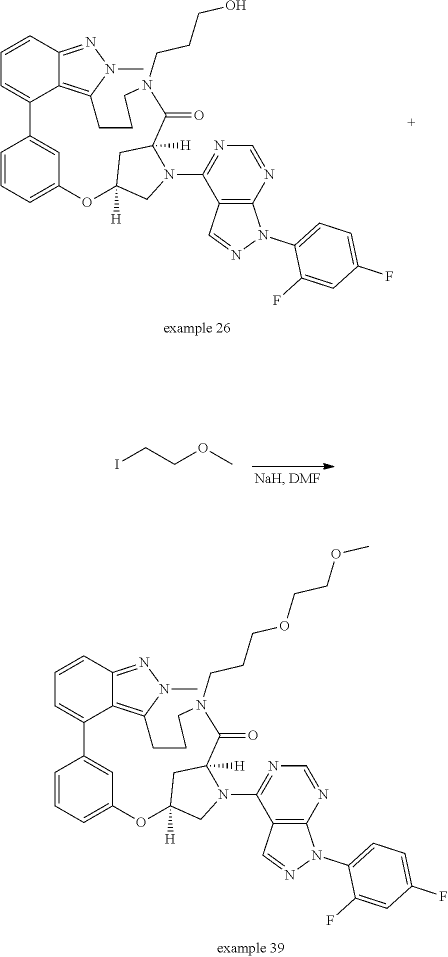

To a mixture of (8S,11S)-10-[1-(2,4-difluorophenyl)pyrazolo[3,4-d]pyrimidin-4-yl]-13-(3-hydroxypropyl)-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one (example 26, 25 mg, 0.04 mmol) and 1-iodo-2-methoxyethane (14 mg, 0.08 mmol) in anhydrous DMF (0.5 mL) was added sodium hydride (5 mg, 0.12 mmol), the resulting mixture was stirred at room temperature for 16 hrs. The reaction mixture was directly purified by prep-HPLC to afford example 39 (0.8 mg) as a white solid, LCMS (M+H)⁺: 723. ¹H NMR (400 MHz, DMSO-d₆) δ=8.68-7.79 (m, 2H), 7.77-7.69 (m, 1H), 7.65-7.57 (m, 1H), 7.54-7.44 (m, 2H), 7.37-7.17 (m, 3H), 6.93 (d, J=7.3 Hz, 1H), 6.85-6.74 (m, 2H), 5.68-5.29 (m, 2H), 4.57-4.22 (m, 2H), 4.07 (s, 3H), 3.83-3.71 (m, 1H), 3.63-3.42 (m, 7H), 3.30-3.20 (m, 4H), 3.18-3.10 (m, 1H), 2.97-2.86 (m, 1H), 2.77-2.56 (m, 2H), 2.44-2.37 (m, 1H), 2.27-2.17 (m, 1H), 1.88-1.78 (m, 1H), 1.70-1.59 (m, 1H), 1.53-1.40 (m, 1H).

### Example 40

### (8S,11S)-10-[1-(2,4-difluorophenyl)pyrazolo[3,4-d]pyrimidin-4-yl]-13-(3-hydroxy-3-methyl-butyl)-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one

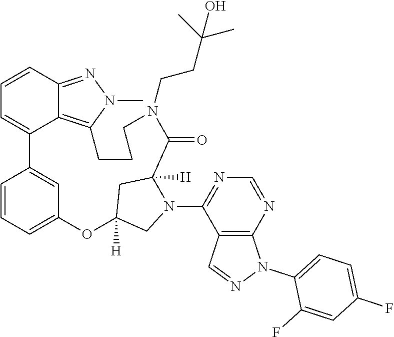 [SMILES: Cn1nc2cccc3c2c1CCCN(CCC(C)(C)O)C(=O)[C@@H]1C[C@@H](CN1c1ncnc2c1cnn2-c1ccc(F)cc1F)Oc1cccc-3c1]

The title compound was prepared according to the following scheme:

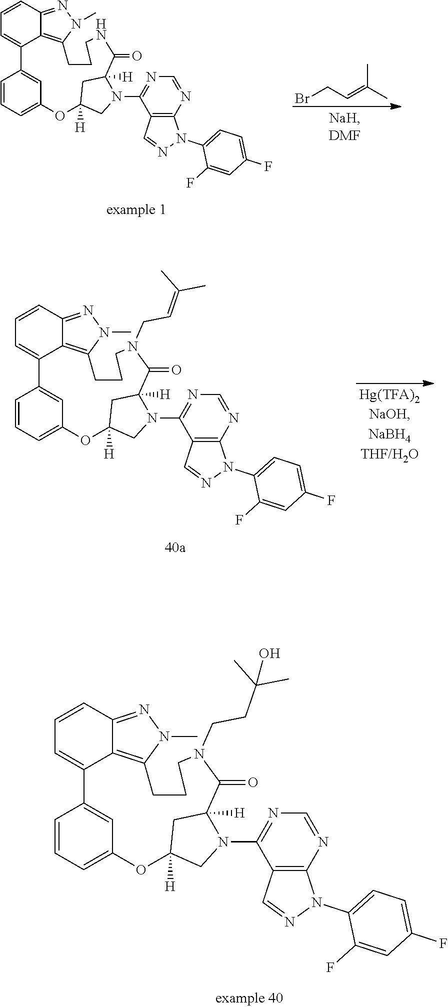

### Step 1: Preparation of (8S,11S)-10-[1-(2,4-difluorophenyl)pyrazolo[3,4-d]pyrimidin-4-yl]-18-methyl-13-(3-methylbut-2-enyl)-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one (Compound 40a)

To a mixture of (8S,11S)-10-[1-(2,4-difluorophenyl)pyrazolo[3,4-d]pyrimidin-4-yl]-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one (example 1, 113 mg, 0.16 mmol) and 1-bromo-3-methyl-but-2-ene (118 mg, 0.79 mmol) in anhydrous DMF (1 mL) was added sodium hydride (32 mg, 0.79 mmol), the resulting mixture was stirred at room temperature for 16 hrs. The reaction mixture was diluted with water, then extracted with DCM for three times. The combined organic layer was washed with water, brine, dried over anhydrous Na₂SO₄, concentrated to give some residue, which was purified by flash column to afford compound 40a (79 mg) as a yellow solid, which was directly used in next step without further purification, LCMS (M+H)⁺: 675.

### Step 2: Preparation of (8S,11S)-10-[1-(2,4-difluorophenyl)pyrazolo[3,4-d]pyrimidin-4-yl]-13-(3-hydroxy-3-methyl-butyl)-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one (Example 40)

To a solution of (8S,11S)-10-[1-(2,4-difluorophenyl)pyrazolo[3,4-d]pyrimidin-4-yl]-18-methyl-13-(3-methylbut-2-enyl)-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one (compound 40a, 79 mg, 0.08 mmol) in THF (1 mL) and water (1 mL) was added mercury(ii) trifluoroacetate (105 mg, 0.25 mmol) in portions at 0° C., the resulting mixture was stirred at 0° C. to room temperature for 16 hrs. Then 3 N aq. NaOH solution (2 mL, 6 mmol) was added and allowed to stir for 2 mins, followed by a solution of NaBH₄ (18.6 mg, 0.49 mmol) in 3 N aq. NaOH (0.4 mL). After being stirred at room temperature for 3 hrs, the reaction mixture was diluted with water, extracted with DCM for three times. The combined organic layer was washed with brine, dried over anhydrous Na₂SO₄, concentrated and purified by prep-HPLC to afford example 40 (9 mg) as a white solid, LCMS (M+H)⁺: 693. ¹H NMR (400 MHz, METHANOL-d₄) δ=8.55-8.32 (m, 1H), 8.25-7.83 (m, 1H), 7.69-7.61 (m, 1H), 7.53-7.45 (m, 2H), 7.32-7.23 (m, 2H), 7.23-7.15 (m, 2H), 7.00-6.90 (m, 2H), 6.82 (d, J=6.7 Hz, 1H), 5.64-5.38 (m, 2H), 4.55 (dd, J=5.3, 11.9 Hz, 1H), 4.48-4.29 (m, 1H), 4.10 (s, 3H), 4.04-3.94 (m, 1H), 3.54-3.33 (m, 2H), 3.04-2.61 (m, 5H), 2.45-2.23 (m, 1H), 1.90-1.71 (m, 1H), 1.67-1.48 (m, 2H), 1.26-1.19 (m, 6H).

### Example 41

### (8S,11S)-10-[1-(2,4-difluorophenyl)pyrazolo[3,4-d]pyrimidin-4-yl]-18-methyl-13-(3-phenylpropyl)-7-oxa-10,13,18,19,26-pentazapentacyclo[15.6.1.1^{2,6}.1^{8,11}.0^{20,24}]hexacosa-1(23),2(26),3,5,17(24),19,21-heptaen-12-one

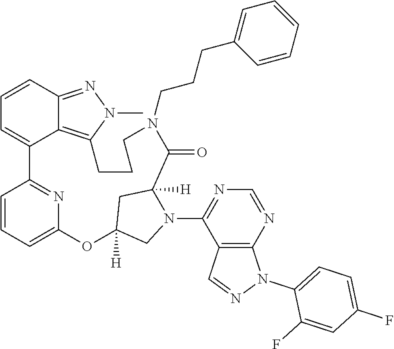 [SMILES: Cn1nc2cccc3c2c1CCCN(CCCc1ccccc1)C(=O)[C@@H]1C[C@@H](CN1c1ncnc2c1cnn2-c1ccc(F)cc1F)Oc1cccc-3n1]

The title compound was prepared according to the following scheme:

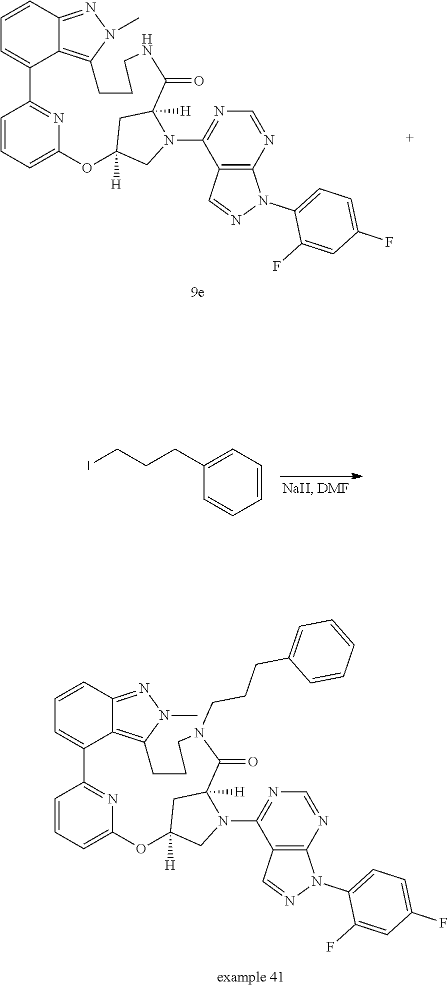 [SMILES: Cn1nc2cccc3c2c1CCCNC(=O)[C@@H]1C[C@@H](CN1c1ncnc2c1cnn2-c1ccc(F)cc1F)Oc1cccc-3n1 ; ICCCc1ccccc1 ; Cn1nc2cccc3c2c1CCCN(CCCc1ccccc1)C(=O)[C@@H]1C[C@@H](CN1c1ncnc2c1cnn2-c1ccc(F)cc1F)Oc1cccc-3n1]

To a solution of (8S,11S)-10-[1-(2,4-difluorophenyl)pyrazolo[3,4-d]pyrimidin-4-yl]-18-methyl-7-oxa-10,13,18,19,26-pentazapentacyclo[15.6.1.1^{2,6}.1^{8,11}.0^{20,24}]hexacosa-1(23),2(26),3,5,17(24),19,21-heptaen-12-one (compound 9e, 44 mg, 0.04 mmol) in anhydrous DMF (1 mL) was added sodium hydride (9 mg, 0.22 mmol) at 0° C., the resulting mixture was stirred at 0° C. for 30 mins. Then (3-iodopropyl)benzene (32 mg, 0.13 mmol) was added and stirred at 0° C. to room temperature for 16 hrs. The reaction mixture was directly purified by prep-HPLC to afford example 41 (4 mg) as a white solid, LCMS (M+H)⁺: 726. ¹H NMR (400 MHz, METHANOL-d₄) δ=8.51-8.19 (m, 2H), 8.01-7.89 (m, 1H), 7.70-7.55 (m, 2H), 7.38-7.09 (m, 10H), 7.02-6.93 (m, 1H), 5.54-5.31 (m, 2H), 4.58-4.33 (m, 2H), 4.20-3.97 (m, 5H), 3.79-3.71 (m, 1H), 3.27-3.18 (m, 1H), 2.96-2.75 (m, 2H), 2.72-2.46 (m, 3H), 2.39-2.26 (m, 2H), 2.08-1.89 (m, 1H), 1.65-1.47 (m, 2H).

### Example 42

### (8S,11S)-10-[1-(2,4-difluorophenyl)pyrazolo[3,4-d]pyrimidin-4-yl]-13-(3-hydroxypropyl)-18-methyl-7,10,13,18,19,26-hexazapentacyclo[15.6.1.1^{2,6}.1^{8,11}.0^{20,24}]hexacosa-1(23),2(26),3,5,17(24),19,21-heptaen-12-one

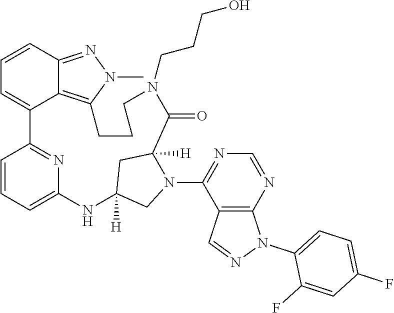 [SMILES: Cn1nc2cccc3c2c1CCCN(CCCO)C(=O)[C@@H]1C[C@@H](CN1c1ncnc2c1cnn2-c1ccc(F)cc1F)Nc1cccc-3n1]

The title compound was prepared in analogy to the preparation of example 12 by using intermediate A5 instead of intermediate A3, intermediate B4 instead of intermediate B13, intermediate C1 instead of intermediate C2 to give example 42 (5 mg) as a white solid, LCMS (M+H)⁺: 665. ¹H NMR (400 MHz, DMSO-d₆) δ=8.66-7.82 (m, 2H), 7.78-7.69 (m, 2H), 7.64-7.48 (m, 2H), 7.37-7.29 (m, 1H), 7.21 (dd, J=6.9, 8.6 Hz, 1H), 7.00-6.91 (m, 1H), 6.89-6.81 (m, 1H), 6.79-6.62 (m, 1H), 5.57-5.27 (m, 2H), 4.81-4.46 (m, 3H), 4.16-4.00 (m, 4H), 3.94-3.77 (m, 1H), 3.64-3.42 (m, 4H), 3.30-3.20 (m, 1H), 3.03-2.88 (m, 1H), 2.83-2.66 (m, 1H), 2.63-2.54 (m, 1H), 2.30-2.14 (m, 2H), 1.87-1.70 (m, 1H), 1.66-1.52 (m, 1H)

### Example 43

### (8S,11S)-10-[1-(4-fluoro-2-methoxy-phenyl)pyrazolo[3,4-d]pyrimidin-4-yl]-18-methyl-13-(3-morpholinopropyl)-7,10,13,18,19,26-hexazapentacyclo[15.6.1.1^{2,6}.1^{8,11}.0^{20,24}]hexacosa-1(23),2(26),3,5,17(24),19,21-heptaen-12-one

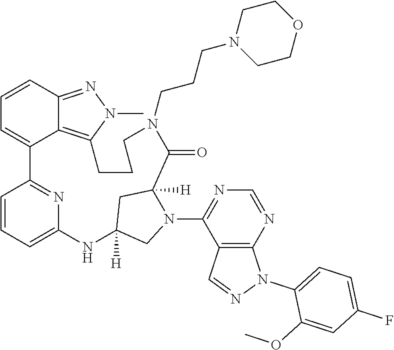 [SMILES: COc1cc(F)ccc1-n1ncc2c(N3C[C@@H]4C[C@H]3C(=O)N(CCCN3CCOCC3)CCCc3c5c(cccc5nn3C)-c3cccc(n3)N4)ncnc21]

The title compound was prepared according to the following scheme:

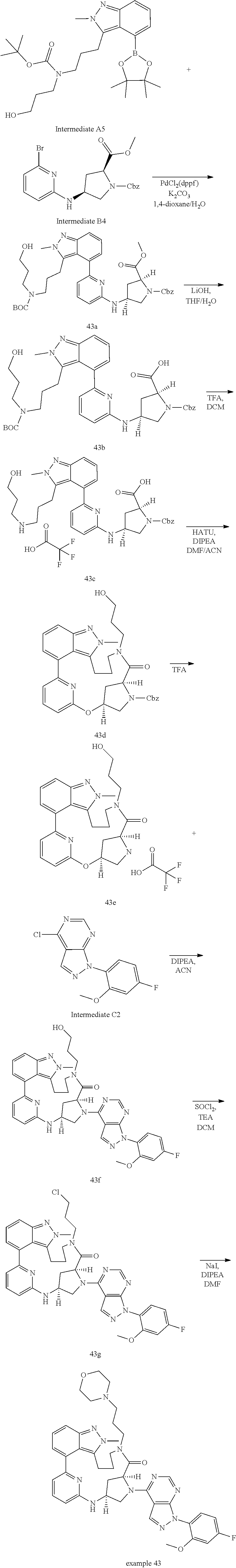

### Step 1-6: Preparation of (8S,11S)-10-[1-(4-fluoro-2-methoxy-phenyl)pyrazolo[3,4-d]pyrimidin-4-yl]-13-(3-hydroxypropyl)-18-methyl-7,10,13,18,19,26-hexazapentacyclo[15.6.1.1^{2,6}.1^{8,11}.0^{20,24}]hexacosa-1(23),2(26),3,5,17(24),19,21-heptaen-12-one (Compound 43f)

Compound 43f was prepared in analogy to the preparation of example 12 by using intermediate A5 instead of intermediate A3, intermediate B4 instead of intermediate B13 to give compound 43f (300 mg) as a brown oil, LCMS (M+H)⁺: 677.

### Step 7: Preparation of (8S,11S)-13-(3-chloropropyl)-10-[1-(4-fluoro-2-methoxy-phenyl)pyrazolo[3,4-d]pyrimidin-4-yl]-18-methyl-7,10,13,18,19,26-hexazapentacyclo[15.6.1.1^{2,6}.1^{8,11}.0^{20,24}]hexacosa-1(23),2(26),3,5,17(24),19,21-heptaen-12-one (Compound 43g)

To a solution of (8S,11S)-10-11-(4-fluoro-2-methoxy-phenyl)pyrazolo[3,4-d]pyrimidin-4-yl]-13-(3-hydroxypropyl)-18-methyl-7,10,13,18,19,26-hexazapentacyclo[15.6.1.1^{2,6}.1^{8,11}.0^{20,24}]hexacosa-1(23),2(26),3,5,17(24),19,21-heptaen-12-one (compound 43f, 200 mg, 0.15 mmol) and TEA (45 mg, 0.45 mmol) in DCM (3 mL) was added thionyl chloride (53 mg, 0.45 mmol) at room temperature under nitrogen, the reaction mixture was stirred at room temperature for 12 hrs. The mixture was diluted with saturated sodium bicarbonate aqueous solution and extracted with EtOAc. The separated organic layer was washed with water, brine, dried over with anhydrous Na₂SO₄, concentrated to give crude compound 43g (110 mg) as a brown solid, LCMS (M+H)⁺: 695.

### Step 3: Preparation of (8S,11S)-10-[1-(4-fluoro-2-methoxy-phenyl)pyrazolo[3,4-d]pyrimidin-4-yl]-18-methyl-13-(3-morpholinopropyl)-7,10,13,18,19,26-hexazapentacyclo[15.6.1.1^{2,6}.1^{8,11}.0^{20,24}]hexacosa-1(23),2(26),3,5,17(24),19,21-heptaen-12-one

### Example 43

A mixture of (8S,11S)-13-(3-chloropropyl)-10-[1-(4-fluoro-2-methoxy-phenyl)pyrazolo[3,4-d]pyrimidin-4-yl]-18-methyl-7,10,13,18,19,26-hexazapentacyclo[15.6.1.1^{2,6}.1^{8,11}.0^{20,24}]hexacosa-1(23),2(26),3,5,17(24),19,21-heptaen-12-one (compound 43g, 200 mg, 0.09 mmol), morpholine (12 mg, 0.13 mmol), DIPEA (22 mg, 0.17 mmol) and sodium iodide (65 mg, 0.43 mmol) in DMF (3 mL) was stirred at 80° C. for 12 hrs. After being cooled to room temperature, the reaction mixture was directly purified by prep-HPLC to afford example 43 (37 mg) as a white solid, LCMS (M+H)⁺: 746. ¹H NMR (500 MHz, DMSO-d₆) δ=8.54-8.27 (m, 1H), 8.18-7.75 (m, 1H), 7.74-7.70 (m, 1H), 7.56-7.52 (m, 1H), 7.47-7.39 (m, 1H), 7.25-7.15 (m, 2H), 7.01-6.92 (m, 2H), 6.89-6.82 (m, 1H), 6.78-6.67 (m, 1H), 5.64-5.48 (m, 1H), 5.41-5.27 (m, 1H), 4.83-4.69 (m, 1H), 4.64-4.49 (m, 1H), 4.12-4.08 (m, 1H), 4.07 (s, 3H), 3.84-3.77 (m, 1H), 3.75-3.71 (m, 3H), 3.65-3.59 (m, 1H), 3.58-3.54 (m, 3H), 3.53-3.44 (m, 1H), 3.35-3.27 (m, 2H), 3.27-3.18 (m, 1H), 3.02-2.94 (m, 1H), 2.78-2.70 (m, 1H), 2.62-2.54 (m, 1H), 2.37 (s, 4H), 2.34-2.27 (m, 2H), 2.26-2.19 (m, 2H), 1.79-1.69 (m, 1H), 1.67-1.57 (m, 1H).

### Example 44

### (8S,11S)-10-[1-(2,4-difluorophenyl)pyrazolo[3,4-d]pyrimidin-4-yl]-21-fluoro-18-methyl-13-(3-morpholinopropyl)-7,10,13,18,19,26-hexazapentacyclo[15.6.1.1^{2,6}.1^{8,11}.0^{20,24}]hexacosa-1(23),2(26),3,5,17(24),19,21-heptaen-12-one

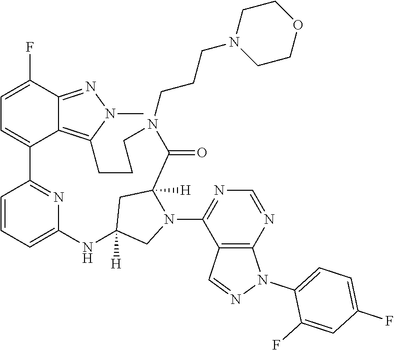 [SMILES: Cn1nc2c(F)ccc3c2c1CCCN(CCCN1CCOCC1)C(=O)[C@@H]1C[C@@H](CN1c1ncnc2c1cnn2-c1ccc(F)cc1F)Nc1cccc-3n1]

The title compound was prepared in analogy to the preparation of example 12 by using intermediate A7 instead of intermediate A3, intermediate B4 instead of intermediate B13 and intermediate C1 instead of intermediate C2 to give example 44 (32 mg) as a white solid, LCMS (M+H)⁺: 752. ¹H NMR (500 MHz, DMSO-d₆) δ=8.64-8.14 (m, 2H), 7.82-7.69 (m, 2H), 7.65-7.55 (m, 1H), 7.36-7.26 (m, 1H), 7.04-6.96 (m, 1H), 6.94-6.79 (m, 2H), 6.77-6.61 (m, 1H), 5.63-5.50 (m, 1H), 5.42-5.30 (m, 1H), 4.80-4.68 (m, 1H), 4.64-4.44 (m, 1H), 4.29-4.00 (m, 4H), 3.93-3.75 (m, 1H), 3.66-3.52 (m, 4H), 3.51-3.38 (m, 2H), 3.26-3.17 (m, 1H), 3.02-2.90 (m, 1H), 2.74 (dt, J=5.0, 13.5 Hz, 1H), 2.65-2.54 (m, 1H), 2.44-2.11 (m, 8H), 1.78-1.68 (m, 1H), 1.63-1.52 (m, 1H).

### Example 45

### (8S,11S)-21-fluoro-10-[1-(4-fluoro-2-methoxy-phenyl)pyrazolo[3,4-d]pyrimidin-4-yl]-18-methyl-13-(3-morpholinopropyl)-7,10,13,18,19,26-hexazapentacyclo[15.6.1.1^{2,6}.1^{8,11}.0^{20,24}]hexacosa-1(23),2(26),3,5,17(24),19,21-heptaen-12-one

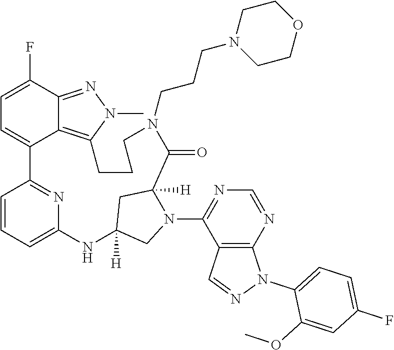 [SMILES: COc1cc(F)ccc1-n1ncc2c(N3C[C@@H]4C[C@H]3C(=O)N(CCCN3CCOCC3)CCCc3c5c(ccc(F)c5nn3C)-c3cccc(n3)N4)ncnc21]

The title compound was prepared in analogy to the preparation of example 12 by using intermediate A7 instead of intermediate A3, intermediate B4 instead of intermediate B13 to give example 45 (77 mg) as a light yellow solid, LCMS (M+H)⁺: 764. ¹H NMR (500 MHz, DMSO-d₆) δ=8.55-7.65 (m, 3H), 7.49-7.38 (m, 1H), 7.25-7.16 (m, 1H), 7.05-6.89 (m, 3H), 6.89-6.81 (m, 1H), 6.80-6.65 (m, 1H), 5.54-5.44 (m, 1H), 5.43-5.31 (m, 1H), 4.80-4.70 (m, 1H), 4.62-4.53 (m, 1H), 4.14-4.04 (m, 4H), 4.01-3.70 (m, 5H), 3.57-3.20 (m, 12H), 3.02-2.91 (m, 1H), 2.79-2.69 (m, 1H), 2.60-2.53 (m, 1H), 2.29-2.15 (m, 2H), 1.86-1.68 (m, 1H), 1.64-1.50 (m, 1H).

### Example 46

### (8S,11S)-10-[1-(4-fluoro-2-methoxy-phenyl)pyrazolo[3,4-d]pyrimidin-4-yl]-4,18-dimethyl-13-(3-morpholinopropyl)-7,10,13,18,19,26-hexazapentacyclo[15.6.1.1^{2,6}.1^{8,11}.0^{20,24}]hexacosa-1(23),2(26),3,5,17(24),19,21-heptaen-12-one

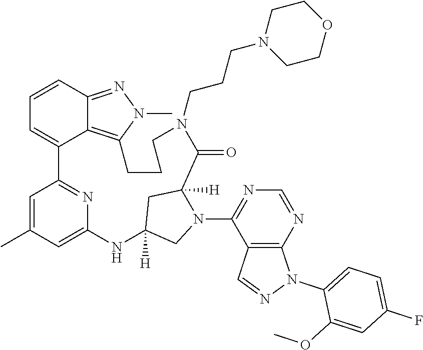 [SMILES: COc1cc(F)ccc1-n1ncc2c(N3C[C@@H]4C[C@H]3C(=O)N(CCCN3CCOCC3)CCCc3c5c(cccc5nn3C)-c3cc(C)cc(n3)N4)ncnc21]

The title compound was prepared in analogy to the preparation of example 43 by using intermediate B5 instead of intermediate B4 to give example 46 (5 mg) as a yellow solid, LCMS (M+H)⁺: 760. ¹H NMR (500 MHz, DMSO-d₆) δ=8.56-8.08 (m, 2H), 7.53 (d, J=8.7 Hz, 1H), 7.48-7.40 (m, 1H), 7.25-7.15 (m, 3H), 7.00-6.91 (m, 2H), 6.75-6.60 (m, 1H), 5.37-5.24 (m, 2H), 4.82-4.69 (m, 1H), 4.66-4.53 (m, 1H), 4.07 (s, 4H), 3.83-3.71 (m, 6H), 3.66-3.48 (m, 8H), 3.28-3.20 (m, 2H), 3.04-2.94 (m, 1H), 2.81-2.70 (m, 1H), 2.60-2.54 (m, 1H), 2.36-2.14 (m, 5H), 2.08-1.57 (m, 2H), 1.52-1.43 (m, 1H).

### Example 47

### (8S,11S)-10-[1-(4-fluoro-2-methoxy-phenyl)pyrazolo[3,4-d]pyrimidin-4-yl]-18-methyl-13-(3-morpholinopropyl)-5,7,10,13,18,19,26-heptazapentacyclo[15.6.1.1^{2,6}.1^{8,11}.0^{20,24}]hexacosa-1(23),2(26),3,5,17(24),19,21-heptaen-12-one

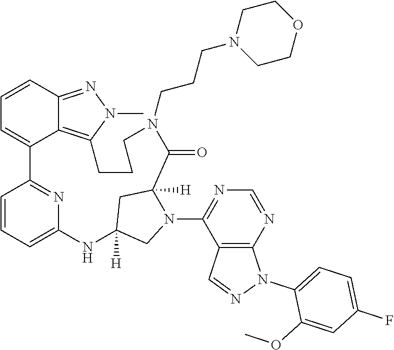 [SMILES: COc1cc(F)ccc1-n1ncc2c(N3C[C@@H]4C[C@H]3C(=O)N(CCCN3CCOCC3)CCCc3c5c(cccc5nn3C)-c3cccc(n3)N4)ncnc21]

The title compound was prepared in analogy to the preparation of example 43 by using intermediate B7 instead of intermediate B4 to give example 47 (2.5 mg) as a yellow solid, LCMS (M+H)⁺: 747.

### Example 48

### Microliter Plate-Based TR-FRET Assay for Binders of STING

This is the competition-binding assay to test the compounds' potency to the C-terminal Domain (CTD) and ligand-binding domain of human stimulator of interferon genes (STING). STING (139-379, Q86WV6, http://www.uniprot.org/uniprot) recombinant protein (in 20 mM Tris, 150 mM NaCl, pH 8.0, and expression in Escherichia coli (E. coli) BL21 (DE3)) with a C-terminal flag-tag was employed for the assay. When Alexa-488 labeled active site probe (refer to patent WO2017/175156 A1) bounds to STING (139-379), it accepts the 485 nm emission from Tb-M2-Flag-STING and results in an increase in fluorescence at 520 nm. Compounds that compete for the probe-binding site will reduce 520 nm signal. The assay was run in proxiplate-384 plus (PerkinElmer, cat: 60150300) containing of 2.5 nM STING, 2.5 nM M2-Tb (Cisbio, 61FG2TLA, Lot: 17 A) and 250 nM Alexa488 probe. Plates were centrifuged for 1 min at 1000 rpm, incubated for 30 min at room temperature, and then measured the dual fluorescence emission at 520 nM/485 nM following 320 nm laser excitation on an Envision plate reader (Perkin-Elmer). The compounds' effect on the STING binding is detected by measuring ratiometric fluorescence from time-resolved FRET. The percentage of inhibition of at each compound concentration is calculated on the basis of their changes of TR-FRET efficiency relative to the change of TR-FRET caused by positive control 2′3′cGAMP (Sigma, cat: SML1229). Table 1 shows data obtained with the disclosed assay for a selection of compounds according to the invention.

TABLE 1
The results of the compounds in TR-FRET assay

| Example NO. | FRET_IC₅₀ (nM) |
|---|---|
| 1 | 0.8 |
| 2 | 2.7 |
| 3 | 0.6 |
| 4 | 0.5 |
| 5 | 3.8 |
| 6 | 5.0 |
| 7 | 0.4 |
| 8 | 0.2 |
| 9 | 17.8 |
| 10 | 5.1 |
| 11 | 21.4 |
| 12 | 18.0 |
| 13 | 4.7 |
| 14 | 4.8 |
| 15 | 1.6 |
| 16 | 3.1 |
| 17 | 2.2 |
| 18 | 12.5 |
| 19 | 13.1 |
| 20 | 1.9 |
| 21 | 2.0 |
| 22 | 0.2 |
| 23 | 1.6 |
| 24 | 8.2 |
| 25 | 18.4 |
| 26 | 2.2 |
| 27 | 14.2 |
| 28 | 0.2 |
| 29 | 1.2 |
| 30 | 2.0 |
| 31 | 7.2 |
| 32 | 3.2 |
| 33 | 1.7 |
| 34 | 7.0 |
| 35 | 0.2 |
| 36 | 1.3 |
| 37 | 1.5 |
| 38 | 0.9 |
| 39 | 5.5 |
| 40 | 8.6 |
| 41 | 2.1 |
| 42 | 5.3 |
| 43 | 3.5 |
| 44 | 6.3 |
| 45 | 3.9 |
| 46 | 7.6 |
| 47 | 7.2 |

### Example 49

hPBMC Cytokine Functional Assay

The inhibition of STING by compounds could be measured by the decrease of IFN-β in hPBMC, which is stimulated by a double stranded DNA virus Baculoviurs (Purchased from Genescript, pCMV-Dest Vector virus generation P2 BV stock virus, Sf-900 II medium with 5% FBS, Lot C9835DK230-2/P4DL001). Frozen hPBMC cells (purchased from Stemcell Technologies, cat number is 70025, Lot 191070803C) were thawed at 37° C. water bath, then cells were re-suspended in 50 ml tube using 20 ml Cell culture medium, and then centrifuged for 5 min at 2000 rpm before the supernatant was removed carefully. The cells were re-suspended in culture medium (RPMI-1640 with 1.5 g/L NaHCO₃, 4.5 g/L glucose, 10 mM HEPES and 1 mM Na-pyruvate, 10% HI-FBS); 65 μL of the cell suspension was dispensed into 384 well plate (70K cells/well) by Multidrop Combi with at least 3 hours incubation at 37° C., 5% CO₂ cell incubator.

Then 4 μL of compound solution per well (final 0.5% DMSO) was transferred by Agilent Bravo followed by infection with 11 μL of Baculovirus at a final MOI of 40. The level of IFN-β secreted in the supernatant of hPBMC was determined after 24 hours of incubation at 37° C. cell incubator by human IFN-β HTRF assay (Cisbio, cat: 62HIFNBPEH) following the manufacture's instruction. CellTiter-Glo kit (Promega, cat: G7573) was applied for the cell viability evaluation using Envision. Table 2 shows data obtained with the disclosed assay for a selection of compounds according to the invention.

TABLE 2
The results of the compounds in hPBMC cytokine functional assay

| Example NO. | hPBMC_IC₅₀ (μM) |
|---|---|
| 1 | 0.486 |
| 2 | 0.594 |
| 3 | 0.288 |
| 4 | 0.413 |
| 5 | 0.308 |
| 6 | 0.588 |
| 7 | 0.325 |
| 8 | 0.397 |
| 9 | 1.584 |
| 11 | 0.803 |
| 12 | 1.407 |
| 13 | 0.224 |
| 14 | 0.255 |
| 15 | 0.140 |
| 16 | 0.384 |
| 18 | 0.491 |
| 19 | 0.976 |
| 20 | 0.346 |
| 21 | 0.380 |
| 22 | 0.264 |
| 23 | 0.279 |
| 24 | 0.711 |
| 25 | 0.549 |
| 26 | 0.103 |
| 27 | 0.023 |
| 28 | 0.184 |
| 29 | 0.031 |
| 30 | 0.173 |
| 31 | 0.517 |
| 32 | 1.105 |
| 33 | 0.806 |
| 34 | 2.486 |
| 35 | 0.183 |
| 36 | 1.568 |
| 38 | 0.465 |
| 40 | 0.708 |
| 41 | 0.048 |
| 43 | 0.292 |
| 44 | 0.321 |
| 45 | 0.569 |

### Metabolic (Liver Microsomal) Stability Assay

The liver microsomal stability assay is used for early assessment of metabolic stability of a test compound in liver microsomes.

Liver microsomes were preincubated with test compound for 10 minutes at 37° C. in 100 mM potassium phosphate buffer, pH 7.4. The reactions were initiated by adding NADPH regenerating system. The final incubation mixtures contained 1 μM test compound, 0.5 mg/mL liver microsomal protein, 1 mM MgCl₂, 1 mM NADP, 1 unit/mL isocitric dehydrogenase and 6 mM isocitric acid in 100 mM potassium phosphate buffer, pH 7.4. After incubation times of 0, 3, 6, 9, 15 and 30 minutes at 37° C., 300 μL of cold acetonitrile (including internal standard) was added to 100 μL incubation mixture to terminate the reaction. Following precipitation and centrifugation, the amount of compound remaining in the samples were determined by LC-MS/MS. Controls of no NADPH regenerating system at zero and 30 minutes were also prepared and analyzed. Table 3 shows data obtained with the disclosed assay for a selection of compounds according to the invention.

TABLE 3
The results of compounds in metabolic stability assay

| | Clearance of human |
|---|---|
| | microsome |
| Example NO. | (mL/min/kg) |
| 4 | 15.0 |
| 6 | 15.7 |
| 7 | 13.3 |
| 8 | 8.9 |
| 9 | 11.6 |
| 10 | 8.3 |
| 11 | 6.1 |
| 12 | 13.2 |
| 16 | 15.9 |
| 18 | 12.4 |
| 19 | 10.0 |
| 22 | 15.5 |
| 24 | 14.9 |
| 31 | 12.4 |
| 32 | 11.5 |
| 34 | 6.1 |
| 36 | 11.9 |
| 40 | 15.6 |
| 42 | 13.3 |
| 43 | 12.0 |
| 44 | 15.8 |
| 45 | 15.9 |
| 46 | 14.9 |
| 47 | 6.1 |

### Example 51

hERG Channel Inhibition Assay

The hERG channel inhibition assay is a highly sensitive measurement that identifies compounds exhibiting hERG inhibition related to cardiotoxicity in vivo. The hERG K⁺ channels were cloned in humans and stably expressed in a CHO (Chinese hamster ovary) cell line. CHO_{hERG} cells (obtained from Roche Palo Alto LLC Cell Core Facilities) were used for patch-clamp (voltage-clamp, whole-cell) experiments. Cells were stimulated by a voltage pattern to activate hERG channels and conduct I_{KhERG} currents (rapid delayed outward rectifier potassium current of the hERG channel). After the cells were stabilized in an internal solution that contains (in mM): KCl 10, KF 100, NaCl 10, HEPES 10, EGTA 20, pH=7.2 with KOH (osmolarity 260-300 mOsm) for a few minutes, the amplitude and kinetics of I_{KhERG} were recorded at a stimulation frequency of 0.1 Hz (6 bpm). Thereafter, the test compound was dissolved in extracellular solution that contains (in mM): NaCl 150, KCl 4, CaCl₂ 1.2, MgCl₂ 1, HEPES 10, glucose 5, pH 7.2-7.4 with NaOH (osmolarity 290-330 mOsm), and was added to the preparation at increasing concentrations. For each concentration, an attempt was made to reach a steady-state effect, usually, this was achieved within 3-10 min at which time the next highest concentration was applied. The amplitude and kinetics of I_{KhERG} are recorded in each concentration of the drug which were compared to the control values (taken as 100%).

### REFERENCES

Redfern W S, Carlsson L, Davis A S, Lynch W G, MacKenzie I, Palethorpe S, Siegl P K, Strang I, Sullivan A T, Wallis R, Camm A J, Hammond T G. 2003; Relationships between preclinical cardiac electrophysiology, clinical QT interval prolongation and torsade de pointes for a broad range of drugs: evidence for a provisional safety margin in drug development. Cardiovasc. Res. 58:32-45
Sanguinetti M C, Tristani-Firouzi M. 2006; hERG potassium channels and cardiac arrhythmia. Nature 440:463-469
Webster R, Leishman D, Walker D. 2002; Towards a drug concentration effect relationship for QT prolongation and torsades de pointes. Curr. Opin. Drug Discov. Devel. 5:116-26.

TABLE 4
The results of compounds in hERG channel inhibition assay

| Example NO. | hERG IC₂₀ (μM) | hERG IC₅₀ (μM) |
|---|---|---|
| 6 | >10.0 | >20.0 |
| 9 | >10.0 | >20.0 |
| 10 | 8.2 | >10.0 |
| 11 | >10.0 | >20.0 |
| 43 | >10.0 | >20.0 |

### Example 52

Single Dose Pharmacokinetics (SDPK) Study in Male C57BL/6J Mice Pharmacokinetic properties of compounds were assessed by single dose PK studies in Male C57BL/6J Mice (vendor: WTLH-BJ). Briefly, two groups of animals were administered a single dose of respective compound intravenously (IV, bolus) at 1 mg/kg or orally (PO, by gavage) at 10 mg/kg. Blood samples were collected via Jugular vein at 5 min (only for IV), 15 min, 30 min, 1 h, 2 h, 4 h, 7 h and 24 h post-dose. Blood samples were placed into pre-chilled commercial EDTA-K2 tubes (vendor: Jiangsu Kangjian medical supplies co., LTD) and centrifuged at 3,200 g for 10 min at 4° C. to separate plasma from the samples. After centrifugation, the resulting plasma was transferred to clean tubes for bioanalysis with LC/MS/MS. The pharmacokinetic parameters were calculated using noncompartmental analysis. The volume of distribution (Vss), half-life (T_{1/2}) and clearance (CL) were obtained based on the plasma concentration-time curve after IV dose. The peak concentration (Cₘₐₓ) was recorded directly from experimental observations after PO dose. The area under the plasma concentration-time curve (AUCₒ₋ₗₐₛₜ) was calculated using the linear trapezoidal linear interpolation rule up to the last detectable concentration. The bioavailability (F) was calculated based on the dose normalized AUCₒ₋ₗₐₛₜ after IV and PO dose. Results of PK parameters following IV and PO administration are given in Table 5.

TABLE 5
PK results for the compounds of this invention

| | | PO | IV | | | | |
|---|---|---|---|---|---|---|---|
| Example | PO Cₘₐₓ | AUCₒ₋ₗₐₛₜ | AUCₒ₋ₗₐₛₜ | CL | Vss | T½ | F |
| NO. | (ng/mL) | (h × ng/mL) | (h × ng/mL) | (mL/min/kg) | (L/kg) | (h) | (%) |
| 6 | 2990 | 4739 | 1118 | 14.5 | 0.9 | 0.8 | 42 |
| 11 | 2783 | 7123 | 1499 | 11.1 | 0.8 | 1.0 | 48 |

1. A compound of formula (I), 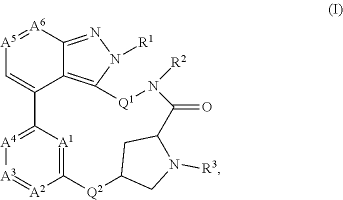
wherein
R¹ is C₁₋₆alkyl;
R² is H, ((C₁₋₆alkylcarbonyl)piperazinyl)C₁₋₆alkyl, (2-oxa-6-azaspiro[3.3]heptanyl)C₁₋₆alkyl, (C₁₋₆alkoxyC₁₋₆alkoxy)C₁₋₆alkyl, (C₁₋₆alkyl)₂aminoC₁₋₆alkyl, (C₁₋₆alkyl-2,5-diazabicyclo[2.2.1]heptanyl)C₁₋₆alkyl, (C₁₋₆alkylpiperazinyl)C₁₋₆alkyl, (C₁₋₆alkylsulfonylpiperazinyl)C₁₋₆alkyl, (C₁₋₆alkyltriazolyl)C₁₋₆alkyl, (pyridinylC₁₋₆alkoxy)C₁₋₆alkyl, 1,2,4-triazolylC₁₋₆alkyl, C₁₋₆alkoxyC₁₋₆alkyl, C₁₋₆alkyl, C₂₋₆alkenyl, C₃₋₇cycloalkylC₁₋₆alkyl, hydroxyC₁₋₆alkyl, imidazolylC₁₋₆alkyl, morpholinylC₁₋₆alkyl, phenylC₁₋₆alkyl, tetrazolylC₁₋₆alkyl or thiazolylC₁₋₆alkyl;
R³ is 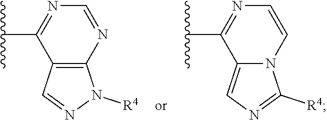
wherein R⁴ is phenyl once, twice or three times substituted by substituents independently selected from halogen and C₁₋₆alkoxy;
Q¹ is C₁₋₆alkylene;
Q² is NH or O;
A¹ is CH or N;
A² is CH or N;
A³ is CR⁵ or N, wherein R⁵ is H, C₁₋₆alkoxy or C₁₋₆alkyl;
A⁴ is CH or N;
A⁵ is CR⁶, wherein R⁶ is H or halogen;
A⁶ is CR⁷, wherein R⁷ is H or halogen;
provided that no more than two of A¹, A², A³ and A⁴ are N simultaneously;
or a pharmaceutically acceptable salt thereof.

2. A compound of formula (Ia) according to claim 1, 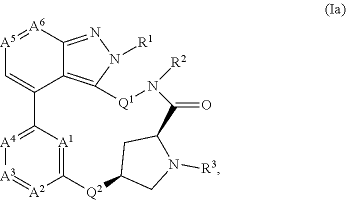
wherein
R¹ is C₁₋₆alkyl;
R² is H, ((C₁₋₆alkylcarbonyl)piperazinyl)C₁₋₆alkyl, (2-oxa-6-azaspiro[3.3]heptanyl)C₁₋₆alkyl, (C₁₋₆alkoxyC₁₋₆alkoxy)C₁₋₆alkyl, (C₁₋₆alkyl)₂aminoC₁₋₆alkyl, (C₁₋₆alkyl-2,5-diazabicyclo[2.2.1]heptanyl)C₁₋₆alkyl, (C₁₋₆alkylpiperazinyl)C₁₋₆alkyl, (C₁₋₆alkylsulfonylpiperazinyl)C₁₋₆alkyl, (C₁₋₆alkyltriazolyl)C₁₋₆alkyl, (pyridinylC₁₋₆alkoxy)C₁₋₆alkyl, 1,2,4-triazolylC₁₋₆alkyl, C₁₋₆alkoxyC₁₋₆alkyl, C₁₋₆alkyl, C₂₋₆alkenyl, C₃₋₇cycloalkylC₁₋₆alkyl, hydroxyC₁₋₆alkyl, imidazolylC₁₋₆alkyl, morpholinylC₁₋₆alkyl, phenylC₁₋₆alkyl, tetrazolylC₁₋₆alkyl or thiazolylC₁₋₆alkyl;
R³ is 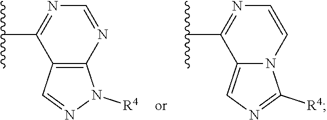
wherein R⁴ is phenyl once, twice or three times substituted by substituents independently selected from halogen and C₁₋₆alkoxy;
Q¹ is C₁₋₆alkylene;
Q² is NH or O;
A¹ is CH or N;
A² is CH or N;
A³ is CR⁵ or N, wherein R⁵ is H, C₁₋₆alkoxy or C₁₋₆alkyl;
A⁴ is CH or N;
A⁵ is CR⁶, wherein R⁶ is H or halogen;
A⁶ is CR⁷, wherein R⁷ is H or halogen;
provided that no more than two of A¹, A², A³ and A⁴ are N simultaneously;
or a pharmaceutically acceptable salt thereof.

3. A compound according to claim 1 or 2, wherein R¹ is methyl.

4. A compound according to any one of claims 1-3, wherein R² is C₁₋₆alkoxyC₁₋₆alkyl, C₁₋₆alkyl, morpholinylC₁₋₆alkyl or phenylC₁₋₆alkyl.

5. A compound according to any one of claims 1-4, wherein R² is 3-methoxypropyl, 3-morpholinylpropyl, 3-phenylpropyl or methyl.

6. A compound according to any one of claims 1-5, wherein R³ is 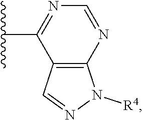
wherein R⁴ is phenyl twice substituted by substituents independently selected from halogen and C₁₋₆alkoxy.

7. A compound according to any one of claims 1-6, wherein R³ is 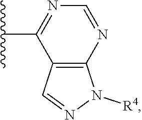
wherein R⁴ is 2,4-difluorophenyl or 4-fluoro-2-methoxy-phenyl.

8. A compound according to any one of claims 1-7, wherein Q¹ is propylene.

9. A compound according to any one of claims 1-8, wherein A³ is CR⁵ or N, wherein R⁵ is H or C₁₋₆alkoxy.

10. A compound according to any one of claims 1-9, wherein A³ is CR⁵ or N, wherein R⁵ is H or methoxy.

11. A compound according to any one of claims 1-10, wherein A⁵ is CH.

12. A compound according to any one of claims 1-11, wherein A⁶ is CH.

13. A compound according to claim 1 or 2, wherein
R¹ is C₁₋₆alkyl;
R² is C₁₋₆alkoxyC₁₋₆alkyl, C₁₋₆alkyl, morpholinylC₁₋₆alkyl or phenylC₁₋₆alkyl;
R³ is 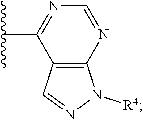
wherein R⁴ is phenyl twice substituted by substituents independently selected from halogen and C₁₋₆alkoxy;
Q¹ is C₁₋₆alkylene;
Q² is NH or O;
A¹ is CH or N;
A² is CH or N;
A³ is CR⁵ or N, wherein R⁵ is H or C₁₋₆alkoxy;
A⁴ is CH or N;
A⁵ is CH;
A⁶ is CH;
provided that no more than two of A¹, A², A³ and A⁴ are N simultaneously;
or a pharmaceutically acceptable salt thereof.

14. A compound according to claim 13, wherein
R¹ is methyl;
R² is 3-methoxypropyl, 3-morpholinylpropyl, 3-phenylpropyl or methyl;
R³ is 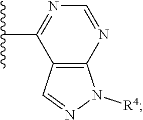
wherein R⁴ is 2,4-difluorophenyl or 4-fluoro-2-methoxy-phenyl;
Q¹ is propylene;
Q² is NH or O;
A¹ is CH or N;
A² is CH or N;
A³ is CR⁵ or N, wherein R⁵ is H or methoxy;
A⁴ is CH or N;
A⁵ is CH;
A⁶ is CH;
provided that no more than two of A¹, A², A³ and A⁴ are N simultaneously;
or a pharmaceutically acceptable salt thereof.

15. A compound selected from:
(8S,11S)-10-[1-(2,4-difluorophenyl)pyrazolo[3,4-d]pyrimidin-4-yl]-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-13,18-dimethyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-13,18-dimethyl-7,10,13,18,19,26-hexazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-13,18-dimethyl-5,7,10,13,18,19,26-heptazapentacyclo[15.6.1.1^{2,6}.1^{8,11}.0^{20,24}]hexacosa-1(23),2,4,6(26),17(24),19,21-heptaen-12-one;
(8S,11S)-10-[3-(2,4-difluorophenyl)imidazo[1,5-a]pyrazin-8-yl]-13,18-dimethyl-5,7,10,13,18,19,26-heptazapentacyclo[15.6.1.1^{2,6}.1^{8,11}.0^{20,24}]hexacosa-1(23),2,4,6(26),17(24),19,21-heptaen-12-one;
(8S,11S)-10-[1-(4-fluoro-2-methoxy-phenyl)pyrazolo[3,4-d]pyrimidin-4-yl]-13,18-dimethyl-5,7,10,13,18,19,26-heptazapentacyclo[15.6.1.1^{2,6}.1^{8,11}.0^{20,24}]hexacosa-1(23),2,4,6(26),17(24),19,21-heptaen-12-one;
(8S,11S)-10-[1-(2,4-difluorophenyl)pyrazolo[3,4-d]pyrimidin-4-yl]-13,18-dimethyl-4,7,10,13,18,19,26-heptazapentacyclo[15.6.1.1^{2,6}.1^{8,11}.0^{20,24}]hexacosa-1(23),2,4,6(26),17(24),19,21-heptaen-12-one;
(8S,11S)-10-[1-(2,4-difluorophenyl)pyrazolo[3,4-d]pyrimidin-4-yl]-13,18-dimethyl-3,7,10,13,18,19,26-heptazapentacyclo[15.6.1.1^{2,6}.1^{8,11}.0^{20,24}]hexacosa-1(23),2,4,6(26),17(24),19,21-heptaen-12-one;
(8S,11S)-10-[1-(2,4-difluorophenyl)pyrazolo[3,4-d]pyrimidin-4-yl]-13,18-dimethyl-7-oxa-10,13,18,19,26-pentazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-18-methyl-7-oxa-3,10,13,18,19-pentazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-13,18-dimethyl-7-oxa-5,10,13,18,19,26-hexazapentacyclo[15.6.1.1^{2,6}.1^{8,11}.0^{20,24}]hexacosa-1(23),2(26),3,5,17(24),19,21-heptaen-12-one;
(8S,11S)-22-fluoro-10-[1-(4-fluoro-2-methoxy-phenyl)pyrazolo[3,4-d]pyrimidin-4-yl]-13,18-dimethyl-7-oxa-5,10,13,18,19,26-hexazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-4-methoxy-13,18-dimethyl-7,10,13,18,19,26-hexazapentacyclo[15.6.1.1^{2,6}.1^{8,11}.0^{20,24}]hexacosa-1(23),2,4,6(26),17(24),19,21-heptaen-12-one;
(8S,11S)-10-[1-(4-fluoro-2-methoxy-phenyl)pyrazolo[3,4-d]pyrimidin-4-yl]-4-methoxy-13,18-dimethyl-7,10,13,18,19,26-hexazapentacyclo[15.6.1.1^{2,6}.1^{8,11}.0^{20,24}]hexacosa-1(23),2,4,6(26),17(24),19,21-heptaen-12-one;
(8S,11S)-13-(cyclopropylmethyl)-10-[1-(2,4-difluorophenyl)pyrazolo[3,4-d]pyrimidin-4-yl]-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-18-methyl-13-[(1-methyl-1,2,4-triazol-3-yl)methyl]-7-oxa-10,13,18,19 tetrazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-18-methyl-13-(thiazol-4-ylmethyl)-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-13-(cyclopropylmethyl)-10-[1-(2,4-difluorophenyl)pyrazolo[3,4-d]pyrimidin-4-yl]-18-methyl-7-oxa-3,5,10,13,18,19-hexazapentacyclo[15.6.1.1^{2,6}.1^{8,11}.0^{20,24}]hexacosa-1(23),2(26),3,5,17(24),19,21-heptaen-12-one;
(8S,11S)-13-(cyclopropylmethyl)-10-[1-(2,4-difluorophenyl)pyrazolo[3,4-d]pyrimidin-4-yl]-18-methyl-7-oxa-5,10,13,18,19,26-hexazapentacyclo[15.6.1.1^{2,6}.1^{8,11}.0^{20,24}]hexacosa-1(23),2(26),3,5,17(24),19,21-heptaen-12-one;
(8S,11S)-13-allyl-10-[1-(2,4-difluorophenyl)pyrazolo[3,4-d]pyrimidin-4-yl]-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-13-(2-methoxyethyl)-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-10-[11-(2,4-difluorophenyl)pyrazolo[3,4-d]pyrimidin-4-yl]-13-(2-hydroxyethyl)-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-18-methyl-13-(2-morpholinoethyl)-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-13-[2-(dimethylamino)ethyl]-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-22-fluoro-18-methyl-13-(2-morpholinoethyl)-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-10-[11-(2,4-difluorophenyl)pyrazolo[3,4-d]pyrimidin-4-yl]-13-(3-hydroxypropyl)-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-10-[11-(2,4-difluorophenyl)pyrazolo[3,4-d]pyrimidin-4-yl]-13-(3-methoxypropyl)-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-18-methyl-13-[2-(4-pyridylmethoxy)ethyl]-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-18-methyl-13-(3-phenylpropyl)-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-18-methyl-13-(3-morpholinopropyl)-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-18-methyl-13-[3-(2-oxa-6-azaspiro[3.3]heptan-6-yl)propyl]-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-18-methyl-13-[3-(4-methylpiperazin-1-yl)propyl]-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-13-[3-(4-acetylpiperazin-1-yl)propyl]-10-[1-(2,4-difluorophenyl)pyrazolo[3,4-d]pyrimidin-4-yl]-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-18-methyl-13-[3-(5-methyl-2,5-diazabicyclo[2.2.1]heptan-2-yl)propyl]-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-18-methyl-13-[3-(4-methylsulfonylpiperazin-1-yl)propyl]-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-18-methyl-13-[3-(tetrazol-1-yl)propyl]-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-18-methyl-13-[3-(1,2,4-triazol-1-yl)propyl]-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-13-(3-imidazol-1-ylpropyl)-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-13-[3-(2-methoxyethoxy)propyl]-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-13-(3-hydroxy-3-methyl-butyl)-18-methyl-7-oxa-10,13,18,19-tetrazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-18-methyl-13-(3-phenylpropyl)-7-oxa-10,13,18,19,26-pentazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-13-(3-hydroxypropyl)-18-methyl-7,10,13,18,19,26-hexazapentacyclo[15.6.1.1^{2,6}.1^{8,11}.0^{20,24}]hexacosa-1(23),2(26),3,5,17(24),19,21-heptaen-12-one;
(8S,11S)-10-[11-(4-fluoro-2-methoxy-phenyl)pyrazolo[3,4-d]pyrimidin-4-yl]-18-methyl-13-(3-morpholinopropyl)-7,10,13,18,19,26-hexazapentacyclo[15.6.1.1^{2,6}.1^{8,11}.0^{20,24}]hexacosa-1(23),2(26),3,5,17(24),19,21-heptaen-12-one;
(8S,11S)-10-[1-(2,4-difluorophenyl)pyrazolo[3,4-d]pyrimidin-4-yl]-21-fluoro-18-methyl-13-(3-morpholinopropyl)-7,10,13,18,19,26-hexazapentacyclo[15.6.1.1^{2,6}.1^{8,11}.0^{20,24}]hexacosa-1(23),2(26),3,5,17(24),19,21-heptaen-12-one;
(8S,11S)-21-fluoro-10-[1-(4-fluoro-2-methoxy-phenyl)pyrazolo[3,4-d]pyrimidin-4-yl]-18-methyl-13-(3-morpholinopropyl)-7,10,13,18,19,26-hexazapentacyclo[15.6.1.1^{2,6}.1^{8,11}.0^{20,24}]hexacosa-1(23),2(26),3,5,17(24),19,21-heptaen-12-one;
(8S,11S)-10-[11-(4-fluoro-2-methoxy-phenyl)pyrazolo[3,4-d]pyrimidin-4-yl]-4,18-dimethyl-13-(3-morpholinopropyl)-7,10,13,18,19,26-hexazapentacyclo[15.6.1.1^{2,6}.1^{8,11}.0^{20,24}]hexacosa-1(23),2(26),3,5,17(24),19,21-heptaen-12-one; and
(8S,11S)-10-[11-(4-fluoro-2-methoxy-phenyl)pyrazolo[3,4-d]pyrimidin-4-yl]-18-methyl-13-(3-morpholinopropyl)-5,7,10,13,18,19,26-heptazapentacyclo[15.6.1.1^{2,6}.1^{8,11}.0^{20,24}]hexacosa-1(23),2(26),3,5,17(24),19,21-heptaen-12-one;
or a pharmaceutically acceptable salt thereof.

16. A process for the preparation of a compound according to any one of claims 1 to 15 comprising the following step:
a) the formation of compound of formula (I) via nucleophilic substitution between compound of formula (VII), 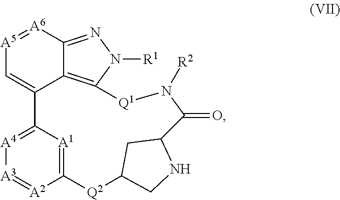
and R³X, in the presence of a base;
wherein the base is DIEA;
R^{a} is H or PG, wherein PG is Boc, Cbz, PMB or phthaloyl; X is halogen.
R¹ to R³, Q¹, Q², A¹ to A⁶ are as defined as in any one of claim 1 to 14.

17. A compound or pharmaceutically acceptable salt according to any one of claims 1 to 15 for use as therapeutically active substance.

18. A pharmaceutical composition comprising a compound in accordance with any one of claims 1 to 15 and a pharmaceutically acceptable excipient.

19. The use of a compound according to any one of claims 1 to 15 for the treatment or prophylaxis of autoimmune diseases, inflammatory diseases, neurological disorders diseases, metabolic diseases, cardiovascular diseases, ocular diseases, or selective types of cancers where overexpression or activation of STING is implicated.

20. The use of a compound according to any one of claims 1 to 15 for the treatment to subjects suffered from an inteferonopathy or auto-inflammatory diseases in which the STING activation are the root-cause of disease pathologies.

21. A compound or pharmaceutically acceptable salt according to any one of claims 1 to 15 for the treatment or prophylaxis of autoimmune diseases, inflammatory diseases, neurological disorders diseases, metabolic diseases, cardiovascular diseases, ocular diseases, or selective types of cancers where overexpression or activation of STING is implicated.

22. The use of a compound according to any one of claims 1 to 15 for the inhibition of STING.

23. The use of a compound according to any one of claims 1 to 15 for the preparation of a medicament for the inhibition of STING.

24. A compound or pharmaceutically acceptable salt according to any one of claims 1 to 15, when manufactured according to a process of claim 16.

25. A method for the treatment or prophylaxis of autoimmune diseases, which method comprises administering a therapeutically effective amount of a compound as defined in any one of claims 1 to 15.
